SEQUENCE LISTING (473 sequences)

SEQ ID NO: 1 - protein, 445 aa, Marsilea vestita
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 2 - protein, 447 aa, Marsilea vestita
MGGADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 3 - protein, 445 aa, Marsilea vestita
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQSRNGSMAGNPSGGLQHDIDVRTKLIHGFHLGFSSGVLARIKVDYYDGTSSDMLGNRGGWPITEQVATGPTLYKVISWSYKVDYGPSRTYGPSVIQLQYTPHMD

SEQ ID NO: 4 - protein, 448 aa, Marsilea vestita
MGGADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMDP

SEQ ID NO: 5 - DNA, 1338 bp, Marsilea vestita
  source 1..1338: mol_type = genomic DNA
atggctgatttggtggccagcaattggaacaaccgtattagagatgctgtgattaaattgagcagcaaaattcctgaggtgggtacagctgtgggcattcttgttgggtttttctggccacaagacaaggtggatatttttgagtccatcaaagccgacattaccaagcttgtgaagaaagaaatacttgactatgaactaagcctgcataaaaatgagttggatgctctcaaggagaccatgaaaagatatgagacggctatgaatcatgagaagggattatttctcaactcatggatcaatgaggctgataggttgagtgtcatctttaggcaatctgccaacaacatccatctcattcaattggtcattagcctggccttgctccaccttactgctctccgtgaaaggtttgactttgggaggagtttatattctgaggacaatacaccacaatggaaggaggatcttgagaacatgtacaagacgtatattgtggactttatccccgacatctacaaaaagtggaaggaatggagaacaaaccaagtagagatctcatcatggcttacacaacgcccgataacacttccaccgttctatacaaatgagtctcatgcaagagttgtggacaatgttacaggtgagataaagaacttctatcaagatttgagtagctcaacatccacatttaacaatatttgtcaagcacataagacacgtatgtgcaatgatgcatattgtgacatggttggttccatatcagccacattttcatatctccaaattttacctgacaacaacaagaactttgcaaagtatgacaaagaagtgtttggtgaagtgttcaaaggaccatattcccttgatctttcttactcaaattattcttatggatatggtaataatgttccaagttttagaataaatcctacaaacaatgattattcgtcaagcgctggcccaattactaaggtaatcatacgagagtggaattctattgatgccatgcaattcatctatcaaggtcggaatgggagcatggcaggtaatccaagtgggggtttgcaacatgacattgatgtgagcaacaaacttatccacggatttcatttggggttctcaagtggtgtacttgcaagaatcaaagttgactactatgacgggacatcctctgatatgcttggaaatcgtggtggttggccaataaccgaacaagttgcaaaaggaccaaccctttataaggtcatctcttggagttacaaagtagattatggtccttcaggcacatatggtccaagtgtaatccaattacaatacacaccacacatggactaa

SEQ ID NO: 6 - DNA, 1344 bp, Marsilea vestita
  source 1..1344: mol_type = genomic DNA
atgggtggtgctgatttggtggccagcaattggaacaaccgtattagagatgctgtgattaaattgagcagcaaaattcctgaggtgggtacagctgtgggcattcttgttgggtttttctggccacaagacaaggtggatatttttgagtccatcaaagccgacattaccaagcttgtgaagaaagaaatacttgactatgaactaagcctgcataaaaatgagttggatgctctcaaggagaccatgaaaagatatgagacggctatgaatcatgagaagggattatttctcaactcatggatcaatgaggctgataggttgagtgtcatctttaggcaatctgccaacaacatccatctcattcaattggtcattagcctggccttgctccaccttactgctctccgtgaaaggtttgactttgggaggagtttatattctgaggacaatacaccacaatggaaggaggatcttgagaacatgtacaagacgtatattgtggactttatccccgacatctacaaaaagtggaaggaatggagaacaaaccaagtagagatctcatcatggcttacacaacgcccgataacacttccaccgttctatacaaatgagtctcatgcaagagttgtggacaatgttacaggtgagataaagaacttctatcaagatttgagtagctcaacatccacatttaacaatatttgtcaagcacataagacacgtatgtgcaatgatgcatattgtgacatggttggttccatatcagccacattttcatatctccaaattttacctgacaacaacaagaactttgcaaagtatgacaaagaagtgtttggtgaagtgttcaaaggaccatattcccttgatctttcttactcaaattattcttatggatatggtaataatgttccaagttttagaataaatcctacaaacaatgattattcgtcaagcgctggcccaattactaaggtaatcatacgagagtggaattctattgatgccatgcaattcatctatcaaggtcggaatgggagcatggcaggtaatccaagtgggggtttgcaacatgacattgatgtgagcaacaaacttatccacggatttcatttggggttctcaagtggtgtacttgcaagaatcaaagttgactactatgacgggacatcctctgatatgcttggaaatcgtggtggttggccaataaccgaacaagttgcaaaaggaccaaccctttataaggtcatctcttggagttacaaagtagattatggtccttcaggcacatatggtccaagtgtaatccaattacaatacacaccacacatggactaa

SEQ ID NO: 7 - DNA, 1338 bp, Marsilea vestita
  source 1..1338: mol_type = genomic DNA
atggctgatttggtggccagcaattggaacaaccgtattagagatgctgtgattaaattgagcagcaaaattcctgaggtgggtacagctgtgggcattcttgttgggtttttctggccacaagacaaggtggatatttttgagtccatcaaagccgacattaccaagcttgtgaagaaagaaatacttgactatgaactaagcctgcataaaaatgagttggatgctctcaaggagaccatgaaaagatatgagacggctatgaatcatgagaagggattatttctcaactcatggatcaatgaggctgataggttgagtgtcatctttaggcaatctgccaacaacatccatctcattcaattggtcattagcctggccttgctccaccttactgctctccgtgaaaggtttgactttgggaggagtttatattctgaggacaatacaccacaatggaaggaggatcttgagaacatgtacaagacgtatattgtggactttatccccgacatctacaaaaagtggaaggaatggagaacaaaccaagtagagatctcatcatggcttacacaacgcccgataacacttccaccgttctatacaaatgagtctcatgcaagagttgtggacaatgttacaggtgagataaagaacttctatcaagatttgagtagctcaacatccacatttaacaatatttgtcaagcacataagacacgtatgtgcaatgatgcatattgtgacatggttggttccatatcagccacattttcatatctccaaattttacctgacaacaacaagaactttgcaaagtatgacaaagaagtgtttggtgaagtgttcaaaggaccatattcccttgatctttcttactcaaattattcttatggatatggtaataatgttccaagttttagaataaatcctacaaacaatgattattcgtcaagcgctggcccaattactaaggtaatcatacgagagtggaactctattgatgccatgcaattcatttatcaaagtcgaaatgggagcatggcaggcaatccaagtgggggtttgcaacatgacattgatgtgagaaccaagcttatccacggatttcatttggggttctcaagtggtgtacttgcaagaatcaaagttgactactacgacgggacatcctctgatatgcttggaaatcgtggtggttggcctataaccgaacaagttgcaacaggaccgaccctttataaggtcatctcttggagttacaaagtagattatggtccttcacgcacatatggtccaagtgtaatccaattacaatacacaccacacatggactaa

SEQ ID NO: 8 - DNA, 1347 bp, Marsilea vestita
  source 1..1347: mol_type = genomic DNA
atgggtggtgctgatttggtggccagcaattggaacaaccgtattagagatgctgtgattaaattgagcagcaaaattcctgaggtgggtacagctgtgggcattcttgttgggtttttctggccacaagacaaggtggatatttttgagtccatcaaagccgacattaccaagcttgtgaagaaagaaatacttgactatgaactaagcctgcataaaaatgagttggatgctctcaaggagaccatgaaaagatatgagacggctatgaatcatgagaagggattatttctcaactcatggatcaatgaggctgataggttgagtgtcatctttaggcaatctgccaacaacatccatctcattcaattggtcattagcctggccttgctccaccttactgctctccgtgaaaggtttgactttgggaggagtttatattctgaggacaatacaccacaatggaaggaggatcttgagaacatgtacaagacgtatattgtggactttatccccgacatctacaaaaagtggaaggaatggagaacaaaccaagtagagatctcatcatggcttacacaacgcccgataacacttccaccgttctatacaaatgagtctcatgcaagagttgtggacaatgttacaggtgagataaagaacttctatcaagatttgagtagctcaacatccacatttaacaatatttgtcaagcacataagacacgtatgtgcaatgatgcatattgtgacatggttggttccatatcagccacattttcatatctccaaattttacctgacaacaacaagaactttgcaaagtatgacaaagaagtgtttggtgaagtgttcaaaggaccatattcccttgatctttcttactcaaattattcttatggatatggtaataatgttccaagttttagaataaatcctacaaacaatgattattcgtcaagcgctggcccaattactaaggtaatcatacgagagtggaattctattgatgccatgcaattcatctatcaaggtcggaatgggagcatggcaggtaatccaagtgggggtttgcaacatgacattgatgtgagcaacaaacttatccacggatttcatttggggttctcaagtggtgtacttgcaagaatcaaagttgactactatgacgggacatcctctgatatgcttggaaatcgtggtggttggccaataaccgaacaagttgcaaaaggaccaaccctttataaggtcatctcttggagttacaaagtagattatggtccttcaggcacatatggtccaagtgtaatccaattacaatacacaccacacatggacccgtaa

SEQ ID NO: 9 - DNA, 1338 bp, Synthetic construct
  source 1..1338: mol_type = other DNA
atggcggacctggtagcgagcaactggaataaccgtatccgcgacgcggtgattaaactcagctctaaaatccctgaggttggtactgccgtcggcatcctggttggctttttctggccgcaagacaaggttgatatctttgaatccatcaaagcggacatcaccaaactggtaaaaaaggagatcctggattatgagctgtccctgcataaaaacgaactggatgctctgaaagaaaccatgaagcgttacgaaactgcaatgaaccacgaaaaaggcctgttcctgaactcttggatcaatgaagcggaccgtctgtctgtcatctttcgtcaatctgcgaataacattcatctgattcagctggtgatcagcctggctctcctgcatctcactgccctgcgtgaacgcttcgacttcggtcgtagcctgtactccgaagataacaccccacagtggaaagaagacctggaaaacatgtacaaaacctatatcgtagacttcatccctgatatctacaagaaatggaaagagtggcgcaccaaccaggtcgaaatctctagctggctgacccagcgcccgatcaccctgccaccgttctataccaatgaaagccacgctcgtgtggtagacaatgtaaccggtgaaatcaaaaatttctaccaggatctgtcttcctctacctccacttttaataacatctgccaggcccacaagacccgcatgtgtaacgatgcctattgtgacatggttggttccatctctgcgactttctcctacctgcagatcctgccggataataacaaaaacttcgcgaaatacgacaaggaagtgttcggcgaggtcttcaaaggtccatattccctggatctgtcttacagcaactactcctacggctatggtaataacgtgccgtctttccgcattaacccgacgaataacgactactcctcttccgcgggcccgatcaccaaagtgattatccgcgaatggaactccatcgatgctatgcagttcatttaccagggccgcaacggttctatggcaggtaatccgagcggcggtctgcagcacgatattgacgtttctaacaaactgattcacggcttccacctgggtttttcctctggcgttctggctcgcatcaaagttgactattacgatggtactagctccgatatgctgggcaaccgtggtggctggccgattaccgaacaggtggcgaaaggtccgaccctgtacaaagtgatcagctggtcttataaagttgattacggcccttccggtacttacggtccgagcgtcattcagctccagtacaccccgcacatggactaa

SEQ ID NO: 10 - DNA, 1344 bp, Synthetic construct
  source 1..1344: mol_type = other DNA
atgggtggcgcggacctggtagcgagcaactggaataaccgtatccgcgacgcggtgattaaactcagctctaaaatccctgaggttggtactgccgtcggcatcctggttggctttttctggccgcaagacaaggttgatatctttgaatccatcaaagcggacatcaccaaactggtaaaaaaggagatcctggattatgagctgtccctgcataaaaacgaactggatgctctgaaagaaaccatgaagcgttacgaaactgcaatgaaccacgaaaaaggcctgttcctgaactcttggatcaatgaagcggaccgtctgtctgtcatctttcgtcaatctgcgaataacattcatctgattcagctggtgatcagcctggctctcctgcatctcactgccctgcgtgaacgcttcgacttcggtcgtagcctgtactccgaagataacaccccacagtggaaagaagacctggaaaacatgtacaaaacctatatcgtagacttcatccctgatatctacaagaaatggaaagagtggcgcaccaaccaggtcgaaatctctagctggctgacccagcgcccgatcaccctgccaccgttctataccaatgaaagccacgctcgtgtggtagacaatgtaaccggtgaaatcaaaaatttctaccaggatctgtcttcctctacctccacttttaataacatctgccaggcccacaagacccgcatgtgtaacgatgcctattgtgacatggttggttccatctctgcgactttctcctacctgcagatcctgccggataataacaaaaacttcgcgaaatacgacaaggaagtgttcggcgaggtcttcaaaggtccatattccctggatctgtcttacagcaactactcctacggctatggtaataacgtgccgtctttccgcattaacccgacgaataacgactactcctcttccgcgggcccgatcaccaaagtgattatccgcgaatggaactccatcgatgctatgcagttcatttaccagggccgcaacggttctatggcaggtaatccgagcggcggtctgcagcacgatattgacgtttctaacaaactgattcacggcttccacctgggtttttcctctggcgttctggctcgcatcaaagttgactattacgatggtactagctccgatatgctgggcaaccgtggtggctggccgattaccgaacaggtggcgaaaggtccgaccctgtacaaagtgatcagctggtcttataaagttgattacggcccttccggtacttacggtccgagcgtcattcagctccagtacaccccgcacatggactaa

SEQ ID NO: 11 - DNA, 1338 bp, Synthetic construct
  source 1..1338: mol_type = other DNA
atggcggacctggtagcgagcaactggaataaccgtatccgcgacgcggtgattaaactcagctctaaaatccctgaggttggtactgccgtcggcatcctggttggctttttctggccgcaagacaaggttgatatctttgaatccatcaaagcggacatcaccaaactggtaaaaaaggagatcctggattatgagctgtccctgcataaaaacgaactggatgctctgaaagaaaccatgaagcgttacgaaactgcaatgaaccacgaaaaaggcctgttcctgaactcttggatcaatgaagcggaccgtctgtctgtcatctttcgtcaatctgcgaataacattcatctgattcagctggtgatcagcctggctctcctgcatctcactgccctgcgtgaacgcttcgacttcggtcgtagcctgtactccgaagataacaccccacagtggaaagaagacctggaaaacatgtacaaaacctatatcgtagacttcatccctgatatctacaagaaatggaaagagtggcgcaccaaccaggtcgaaatctctagctggctgacccagcgcccgatcaccctgccaccgttctataccaatgaaagccacgctcgtgtggtagacaatgtaaccggtgaaatcaaaaatttctaccaggatctgtcttcctctacctccacttttaataacatctgccaggcccacaagacccgcatgtgtaacgatgcctattgtgacatggttggttccatctctgcgactttctcctacctgcagatcctgccggataataacaaaaacttcgcgaaatacgacaaggaagtgttcggcgaggtcttcaaaggtccatattccctggatctgtcttacagcaactactcctacggctatggtaataacgtgccgtctttccgcattaacccgacgaataacgactactcctcttccgcgggcccgatcaccaaagtgattatccgcgaatggaactccatcgatgctatgcagttcatttaccagagccgcaacggttctatggcaggtaatccgagcggcggtctgcagcacgatattgacgttcgcactaaactgattcacggcttccacctgggtttttcctctggcgttctggctcgcatcaaagttgactattacgatggtactagctccgatatgctgggcaaccgtggtggctggccgattaccgaacaggtggcgactggtccgaccctgtacaaagtgatcagctggtcttataaagttgattacggcccttcccgcacttacggtccgagcgtcattcagctccagtacaccccgcacatggactaa

SEQ ID NO: 12 - DNA, 1347 bp, Synthetic construct
  source 1..1347: mol_type = other DNA
atgggtggcgcggacctggtagcgagcaactggaataaccgtatccgcgacgcggtgattaaactcagctctaaaatccctgaggttggtactgccgtcggcatcctggttggctttttctggccgcaagacaaggttgatatctttgaatccatcaaagcggacatcaccaaactggtaaaaaaggagatcctggattatgagctgtccctgcataaaaacgaactggatgctctgaaagaaaccatgaagcgttacgaaactgcaatgaaccacgaaaaaggcctgttcctgaactcttggatcaatgaagcggaccgtctgtctgtcatctttcgtcaatctgcgaataacattcatctgattcagctggtgatcagcctggctctcctgcatctcactgccctgcgtgaacgcttcgacttcggtcgtagcctgtactccgaagataacaccccacagtggaaagaagacctggaaaacatgtacaaaacctatatcgtagacttcatccctgatatctacaagaaatggaaagagtggcgcaccaaccaggtcgaaatctctagctggctgacccagcgcccgatcaccctgccaccgttctataccaatgaaagccacgctcgtgtggtagacaatgtaaccggtgaaatcaaaaatttctaccaggatctgtcttcctctacctccacttttaataacatctgccaggcccacaagacccgcatgtgtaacgatgcctattgtgacatggttggttccatctctgcgactttctcctacctgcagatcctgccggataataacaaaaacttcgcgaaatacgacaaggaagtgttcggcgaggtcttcaaaggtccatattccctggatctgtcttacagcaactactcctacggctatggtaataacgtgccgtctttccgcattaacccgacgaataacgactactcctcttccgcgggcccgatcaccaaagtgattatccgcgaatggaactccatcgatgctatgcagttcatttaccagggccgcaacggttctatggcaggtaatccgagcggcggtctgcagcacgatattgacgtttctaacaaactgattcacggcttccacctgggtttttcctctggcgttctggctcgcatcaaagttgactattacgatggtactagctccgatatgctgggcaaccgtggtggctggccgattaccgaacaggtggcgaaaggtccgaccctgtacaaagtgatcagctggtcttataaagttgattacggcccttccggtacttacggtccgagcgtcattcagctccagtacaccccgcacatggacccgtaa

SEQ ID NO: 13 - DNA, 1338 bp, Synthetic construct
  source 1..1338: mol_type = other DNA
atggcggacctggtcgcctcgaactggaacaaccgcatccgcgacgccgtgatcaagctctcgtcgaagatcccggaggtggggacggccgtgggtatcctggtggggttcttctggccccaggataaggtggatatcttcgagtccatcaaggcggacatcacgaagctcgttaagaaggagatactggactacgagctgtccctccacaagaacgagctggacgcgctcaaggagaccatgaagcgctacgagactgctatgaaccacgagaagggcctcttcctgaactcgtggatcaacgaggcggacaggctctccgtcatcttccgccagtccgccaacaacatccacctcatccagctcgtgatctccctcgccctcctgcacctcaccgcgctgcgcgagcgcttcgacttcggccgctccctgtacagcgaggacaacaccccgcagtggaaggaggacctggagaacatgtataagacgtacatcgtcgacttcatcccggacatctacaagaagtggaaggagtggcgtaccaaccaggtggagatctcttcgtggctcactcagcgcccgatcacgcttccgcccttctacacgaatgaaagccacgcccgggtcgtcgacaacgtcaccggcgagatcaagaacttctaccaggacctctcgtcctccacatccaccttcaacaacatctgccaggcgcacaagacccgcatgtgcaacgacgcgtactgcgacatggtcggctcgatttcggccacattctcctacctccagattctccccgacaacaacaagaacttcgcaaagtacgacaaggaggtcttcggggaggtgttcaagggcccgtactccctggacctgagctactccaactacagctacggctacggcaacaacgtcccctccttccgcatcaaccccacgaataacgactactcctcttcggccggccctatcaccaaggtcatcatcagggagtggaactccatcgacgccatgcagttcatctaccaggggcgcaacggctcgatggccggcaacccgtctggcggccttcagcacgacatcgacgtgtctaacaagctcatccacggcttccatctaggcttctcctccggcgtgctggcccggatcaaggtggactactacgacggcacctcctccgacatgctcggcaaccgcggcggttggccgatcacggagcaggtggcgaagggcccgaccctgtacaaggtcatctcctggagctacaaggtcgactacgggccctccggtacctacggcccgtcggtcatccagctccagtacacgccgcacatggactag

SEQ ID NO: 14 - DNA, 1344 bp, Synthetic construct
  source 1..1344: mol_type = other DNA
atggggggcgcggacctggtcgcctcgaactggaacaaccgcatccgcgacgccgtgatcaagctctcgtcgaagatcccggaggtggggacggccgtgggtatcctggtggggttcttctggccccaggataaggtggatatcttcgagtccatcaaggcggacatcacgaagctcgttaagaaggagatactggactacgagctgtccctccacaagaacgagctggacgcgctcaaggagaccatgaagcgctacgagactgctatgaaccacgagaagggcctcttcctgaactcgtggatcaacgaggcggacaggctctccgtcatcttccgccagtccgccaacaacatccacctcatccagctcgtgatctccctcgccctcctgcacctcaccgcgctgcgcgagcgcttcgacttcggccgctccctgtacagcgaggacaacaccccgcagtggaaggaggacctggagaacatgtataagacgtacatcgtcgacttcatcccggacatctacaagaagtggaaggagtggcgtaccaaccaggtggagatctcttcgtggctcactcagcgcccgatcacgcttccgcccttctacacgaatgaaagccacgcccgggtcgtcgacaacgtcaccggcgagatcaagaacttctaccaggacctctcgtcctccacatccaccttcaacaacatctgccaggcgcacaagacccgcatgtgcaacgacgcgtactgcgacatggtcggctcgatttcggccacattctcctacctccagattctccccgacaacaacaagaacttcgcaaagtacgacaaggaggtcttcggggaggtgttcaagggcccgtactccctggacctgagctactccaactacagctacggctacggcaacaacgtcccctccttccgcatcaaccccacgaataacgactactcctcttcggccggccctatcaccaaggtcatcatcagggagtggaactccatcgacgccatgcagttcatctaccaggggcgcaacggctcgatggccggcaacccgtctggcggccttcagcacgacatcgacgtgtctaacaagctcatccacggcttccatctaggcttctcctccggcgtgctggcccggatcaaggtggactactacgacggcacctcctccgacatgctcggcaaccgcggcggttggccgatcacggagcaggtggcgaagggcccgaccctgtacaaggtcatctcctggagctacaaggtcgactacgggccctccggtacctacggcccgtcggtcatccagctccagtacacgccgcacatggactag

SEQ ID NO: 15 - DNA, 1338 bp, Synthetic construct
  source 1..1338: mol_type = other DNA
atggcggacctggtcgcctcgaactggaacaaccgcatccgcgacgccgtgatcaagctctcgtcgaagatcccggaggtggggacggccgtgggtatcctggtggggttcttctggccccaggataaggtggatatcttcgagtccatcaaggcggacatcacgaagctcgttaagaaggagatactggactacgagctgtccctccacaagaacgagctggacgcgctcaaggagaccatgaagcgctacgagactgctatgaaccacgagaagggcctcttcctgaactcgtggatcaacgaggcggacaggctctccgtcatcttccgccagtccgccaacaacatccacctcatccagctcgtgatctccctcgccctcctgcacctcaccgcgctgcgcgagcgcttcgacttcggccgctccctgtacagcgaggacaacaccccgcagtggaaggaggacctggagaacatgtataagacgtacatcgtcgacttcatcccggacatctacaagaagtggaaggagtggcgtaccaaccaggtggagatctcttcgtggctcactcagcgcccgatcacgcttccgcccttctacacgaatgaaagccacgcccgggtcgtcgacaacgtcaccggcgagatcaagaacttctaccaggacctctcgtcctccacatccaccttcaacaacatctgccaggcgcacaagacccgcatgtgcaacgacgcgtactgcgacatggtcggctcgatttcggccacattctcctacctccagattctccccgacaacaacaagaacttcgcaaagtacgacaaggaggtcttcggggaggtgttcaagggcccgtactccctggacctgagctactccaactacagctacggctacggcaacaacgtcccctccttccgcatcaaccccacgaataacgactactcctcttcggccggccctatcaccaaggtcatcatcagggagtggaactccatcgacgccatgcagttcatctaccagtcccgcaacggctcgatggccggcaacccgtctggcggccttcagcacgacatcgacgtgcgcaccaagctcatccacggcttccatctaggcttctcctccggcgtgctggcccggatcaaggtggactactacgacggcacctcctccgacatgctcggcaaccgcggcggttggccgatcacggagcaggtggcgaccggcccgaccctgtacaaggtcatctcctggagctacaaggtcgactacgggccctcccgcacctacggcccgtcggtcatccagctccagtacacgccgcacatggactag

SEQ ID NO: 16 - DNA, 1347 bp, Synthetic construct
  source 1..1347: mol_type = other DNA
atggggggcgcggacctggtcgcctcgaactggaacaaccgcatccgcgacgccgtgatcaagctctcgtcgaagatcccggaggtggggacggccgtgggtatcctggtggggttcttctggccccaggataaggtggatatcttcgagtccatcaaggcggacatcacgaagctcgttaagaaggagatactggactacgagctgtccctccacaagaacgagctggacgcgctcaaggagaccatgaagcgctacgagactgctatgaaccacgagaagggcctcttcctgaactcgtggatcaacgaggcggacaggctctccgtcatcttccgccagtccgccaacaacatccacctcatccagctcgtgatctccctcgccctcctgcacctcaccgcgctgcgcgagcgcttcgacttcggccgctccctgtacagcgaggacaacaccccgcagtggaaggaggacctggagaacatgtataagacgtacatcgtcgacttcatcccggacatctacaagaagtggaaggagtggcgtaccaaccaggtggagatctcttcgtggctcactcagcgcccgatcacgcttccgcccttctacacgaatgaaagccacgcccgggtcgtcgacaacgtcaccggcgagatcaagaacttctaccaggacctctcgtcctccacatccaccttcaacaacatctgccaggcgcacaagacccgcatgtgcaacgacgcgtactgcgacatggtcggctcgatttcggccacattctcctacctccagattctccccgacaacaacaagaacttcgcaaagtacgacaaggaggtcttcggggaggtgttcaagggcccgtactccctggacctgagctactccaactacagctacggctacggcaacaacgtcccctccttccgcatcaaccccacgaataacgactactcctcttcggccggccctatcaccaaggtcatcatcagggagtggaactccatcgacgccatgcagttcatctaccaggggcgcaacggctcgatggccggcaacccgtctggcggccttcagcacgacatcgacgtgtctaacaagctcatccacggcttccatctaggcttctcctccggcgtgctggcccggatcaaggtggactactacgacggcacctcctccgacatgctcggcaaccgcggcggttggccgatcacggagcaggtggcgaagggcccgaccctgtacaaggtcatctcctggagctacaaggtcgactacgggccctccggtacctacggcccgtcggtcatccagctccagtacacgccgcacatggacccctag

SEQ ID NO: 17 - protein, 12 aa, Synthetic construct
MHHHHHHGGGGS

SEQ ID NO: 18 - DNA, 372 bp, synthetic construct
  source 1..372: mol_type = other DNA
atggcctcaatatcctcctccgtcgcaacagtctcaagaaccgcccctgctcaggccaacatggtggctccgttcaccggccttaagtccaacgccgccttccccaccaccaagaaggctaacgacttctccacccttcccagcaacggtggaagagttcagtgtatgcaggtgtggccggcctacggcaacaagaagttcgagacgctgtcgtacctgccgccgctgtctatggcgcccaccgtgatgatggcctcgtcggccaccgccgtcgctccgttccaggggctcaagtccaccgccagcctccccgtcgcccgccgctcctccagaagcctcggcaacgtcagcaacggcggaagaatccggtgt

SEQ ID NO: 19 - protein, 124 aa, synthetic construct
MASISSSVATVSRTAPAQANMVAPFTGLKSNAAFPTTKKANDFSTLPSNGGRVQCMQVWPAYGNKKFETLSYLPPLSMAPTVMMASSATAVAPFQGLKSTASLPVARRSSRSLGNVSNGGRIRC

SEQ ID NO: 20 - DNA, 156 bp, synthetic construct
  source 1..156: mol_type = other DNA
atggcttgctccacccacctcctgtcccagagcctctacccgctcaaccgcgctaacccggcagccgcgcgcgggcacctccggttccaggcttctcccagtgtgaggctcggctccggcaccagtcgccgccgggcgctgggcctgagagtcgcc

SEQ ID NO: 21 - protein, 52 aa, synthetic construct
MACSTHLLSQSLYPLNRANPAAARGHLRFQASPSVRLGSGTSRRRALGLRVA

SEQ ID NO: 22 - DNA, 228 bp, synthetic construct
  source 1..228: mol_type = other DNA
atggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgc

SEQ ID NO: 23 - protein, 76 aa, synthetic construct
MAQVSRICNGVQNPSLISNLSKSSQRKSPLSVSLKTQQHPRAYPISSSWGLKKSGMTLIGSELRPLKVMSSVSTAC

SEQ ID NO: 24 - DNA, 174 bp, synthetic construct
  source 1..174: mol_type = other DNA
atggcctccctcgcctcggcctcaacctcactcctcttcccgcaggccacctcatccaggagccgcgtccggctctccacctccctgggcttctccgcgcagccggcgcggctgcggagccgggcggcggcggcgggcgggcagcggcgcgggcgcctgctggtggtgcgcgcg

SEQ ID NO: 25 - protein, 58 aa, synthetic construct
MASLASASTSLLFPQATSSRSRVRLSTSLGFSAQPARLRSRAAAAGGQRRGRLLVVRA

SEQ ID NO: 26 - protein, 569 aa, synthetic construct
MASISSSVATVSRTAPAQANMVAPFTGLKSNAAFPTTKKANDFSTLPSNGGRVQCMQVWPAYGNKKFETLSYLPPLSMAPTVMMASSATAVAPFQGLKSTASLPVARRSSRSLGNVSNGGRIRCMADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 27 - protein, 497 aa, synthetic construct
MACSTHLLSQSLYPLNRANPAAARGHLRFQASPSVRLGSGTSRRRALGLRVAMADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 28 - protein, 521 aa, synthetic construct
MAQVSRICNGVQNPSLISNLSKSSQRKSPLSVSLKTQQHPRAYPISSSWGLKKSGMTLIGSELRPLKVMSSVSTACMADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 29 - protein, 503 aa, synthetic construct
MASLASASTSLLFPQATSSRSRVRLSTSLGFSAQPARLRSRAAAAGGQRRGRLLVVRAMADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 30 - protein, 445 aa, synthetic construct
MSDLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 31 - protein, 445 aa, synthetic construct
MAALVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 32 - protein, 445 aa, synthetic construct
MADAVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 33 - protein, 445 aa, synthetic construct
MADLAASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 34 - protein, 445 aa, synthetic construct
MADLVSSNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 35 - protein, 445 aa, synthetic construct
MADLVAANWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 36 - protein, 445 aa, synthetic construct
MADLVASAWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 37 - protein, 445 aa, synthetic construct
MADLVASNANNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 38 - protein, 445 aa, synthetic construct
MADLVASNWANRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 39 - protein, 445 aa, synthetic construct
MADLVASNWNARIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 40 - protein, 445 aa, synthetic construct
MADLVASNWNNAIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 41 - protein, 445 aa, synthetic construct
MADLVASNWNNRARDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 42 - protein, 445 aa, synthetic construct
MADLVASNWNNRIADAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 43 - protein, 445 aa, synthetic construct
MADLVASNWNNRIRAAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 44 - protein, 445 aa, synthetic construct
MADLVASNWNNRIRDSVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 45 - protein, 445 aa, synthetic construct
MADLVASNWNNRIRDAAIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 46 - protein, 445 aa, synthetic construct
MADLVASNWNNRIRDAVAKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 47 - protein, 445 aa, synthetic construct
MADLVASNWNNRIRDAVIALSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 48 - protein, 445 aa, synthetic construct
MADLVASNWNNRIRDAVIKASSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 49 - protein, 445 aa, synthetic construct
MADLVASNWNNRIRDAVIKLASKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 50 - protein, 445 aa, synthetic construct
MADLVASNWNNRIRDAVIKLSAKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 51 - protein, 445 aa, synthetic construct
MADLVASNWNNRIRDAVIKLSSAIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 52 - protein, 445 aa, synthetic construct
MADLVASNWNNRIRDAVIKLSSKAPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 53 - protein, 445 aa, synthetic construct
MADLVASNWNNRIRDAVIKLSSKIAEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 54 - protein, 445 aa, synthetic construct
MADLVASNWNNRIRDAVIKLSSKIPAVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 55 - protein, 445 aa, synthetic construct
MADLVASNWNNRIRDAVIKLSSKIPEAGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 56 - protein, 445 aa, synthetic construct
MADLVASNWNNRIRDAVIKLSSKIPEVATAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 57 - protein, 445 aa, synthetic construct
MADLVASNWNNRIRDAVIKLSSKIPEVGAAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 58 - protein, 445 aa, synthetic construct
MADLVASNWNNRIRDAVIKLSSKIPEVGTSVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 59 - protein, 445 aa, synthetic construct
MADLVASNWNNRIRDAVIKLSSKIPEVGTAAGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 60 - protein, 445 aa, synthetic construct
MADLVASNWNNRIRDAVIKLSSKIPEVGTAVAILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 61 - protein, 445 aa, synthetic construct
MADLVASNWNNRIRDAVIKLSSKIPEVGTAVGALVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 62 - protein, 445 aa, synthetic construct
MADLVASNWNNRIRDAVIKLSSKIPEVGTAVGIAVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 63 - protein, 445 aa, synthetic construct
MADLVASNWNNRIRDAVIKLSSKIPEVGTAVGILAGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 64 - protein, 445 aa, synthetic construct
MADLVASNWNNRIRDAVIKLSSKIPEVGTAVGILVAFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 65 - protein, 445 aa, synthetic construct
MADLVASNWNNRIRDAVIKLSSKIPEVGTAVGILVGAFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 66 - protein, 445 aa, synthetic construct
MADLVASNWNNRIRDAVIKLSSKIPEVGTAVGILVGFAWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 67 - protein, 445 aa, synthetic construct
MADLVASNWNNRIRDAVIKLSSKIPEVGTAVGILVGFFAPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 68 - protein, 445 aa, synthetic construct
MADLVASNWNNRIRDAVIKLSSKIPEVGTAVGILVGFFWAQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 69 - protein, 445 aa, synthetic construct
MADLVASNWNNRIRDAVIKLSSKIPEVGTAVGILVGFFWPADKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 70 - protein, 445 aa, synthetic construct
MADLVASNWNNRIRDAVIKLSSKIPEVGTAVGILVGFFWPQAKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 71 - protein, 445 aa, synthetic construct
MADLVASNWNNRIRDAVIKLSSKIPEVGTAVGILVGFFWPQDAVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 72 - protein, 445 aa, synthetic construct
MADLVASNWNNRIRDAVIKLSSKIPEVGTAVGILVGFFWPQDKADIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 73 - protein, 445 aa, synthetic construct
MADLVASNWNNRIRDAVIKLSSKIPEVGTAVGILVGFFWPQDKVAIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 74 - protein, 445 aa, synthetic construct
MADLVASNWNNRIRDAVIKLSSKIPEVGTAVGILVGFFWPQDKVDAFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 75 - protein, 445 aa, synthetic construct
MADLVASNWNNRIRDAVIKLSSKIPEVGTAVGILVGFFWPQDKVDIAESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 76 - protein, 445 aa, synthetic construct
MADLVASNWNNRIRDAVIKLSSKIPEVGTAVGILVGFFWPQDKVDIFASIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 77 - protein, 445 aa, synthetic construct
MADLVASNWNNRIRDAVIKLSSKIPEVGTAVGILVGFFWPQDKVDIFEAIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 78 - protein, 445 aa, synthetic construct
MADLVASNWNNRIRDAVIKLSSKIPEVGTAVGILVGFFWPQDKVDIFESAKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 79 - protein, 445 aa, synthetic construct
MADLVASNWNNRIRDAVIKLSSKIPEVGTAVGILVGFFWPQDKVDIFESIAADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 80 - protein, 445 aa, synthetic construct
MADLVASNWNNRIRDAVIKLSSKIPEVGTAVGILVGFFWPQDKVDIFESIKSDITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 81 - protein, 445 aa, synthetic construct
MADLVASNWNNRIRDAVIKLSSKIPEVGTAVGILVGFFWPQDKVDIFESIKAAITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 82 - protein, 445 aa, synthetic construct
MADLVASNWNNRIRDAVIKLSSKIPEVGTAVGILVGFFWPQDKVDIFESIKADATKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 83 - protein, 445 aa, synthetic construct
MADLVASNWNNRIRDAVIKLSSKIPEVGTAVGILVGFFWPQDKVDIFESIKADIAKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 84 - protein, 445 aa, synthetic construct
MADLVASNWNNRIRDAVIKLSSKIPEVGTAVGILVGFFWPQDKVDIFESIKADITALVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 85 - protein, 445 aa, synthetic construct
MADLVASNWNNRIRDAVIKLSSKIPEVGTAVGILVGFFWPQDKVDIFESIKADITKAVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 86 - protein, 445 aa, synthetic construct
MADLVASNWNNRIRDAVIKLSSKIPEVGTAVGILVGFFWPQDKVDIFESIKADITKLAKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 87 - protein, 445 aa, synthetic construct
MADLVASNWNNRIRDAVIKLSSKIPEVGTAVGILVGFFWPQDKVDIFESIKADITKLVAKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 88 - protein, 445 aa, synthetic construct
MADLVASNWNNRIRDAVIKLSSKIPEVGTAVGILVGFFWPQDKVDIFESIKADITKLVKAEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 89 - protein, 445 aa, synthetic construct
MADLVASNWNNRIRDAVIKLSSKIPEVGTAVGILVGFFWPQDKVDIFESIKADITKLVKKAILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 90 - protein, 445 aa, synthetic construct
MADLVASNWNNRIRDAVIKLSSKIPEVGTAVGILVGFFWPQDKVDIFESIKADITKLVKKEALDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 91 - protein, 445 aa, synthetic construct
MADLVASNWNNRIRDAVIKLSSKIPEVGTAVGILVGFFWPQDKVDIFESIKADITKLVKKEIADYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 92 - protein, 445 aa, synthetic construct
MADLVASNWNNRIRDAVIKLSSKIPEVGTAVGILVGFFWPQDKVDIFESIKADITKLVKKEILAYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 93 - protein, 445 aa, synthetic construct
MADLVASNWNNRIRDAVIKLSSKIPEVGTAVGILVGFFWPQDKVDIFESIKADITKLVKKEILDAELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 94 - protein, 445 aa, synthetic construct
MADLVASNWNNRIRDAVIKLSSKIPEVGTAVGILVGFFWPQDKVDIFESIKADITKLVKKEILDYALSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 95 - protein, 445 aa, synthetic construct
MADLVASNWNNRIRDAVIKLSSKIPEVGTAVGILVGFFWPQDKVDIFESIKADITKLVKKEILDYEASLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 96 - protein, 445 aa, synthetic construct
MADLVASNWNNRIRDAVIKLSSKIPEVGTAVGILVGFFWPQDKVDIFESIKADITKLVKKEILDYELALHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 97 - protein, 445 aa, synthetic construct
MADLVASNWNNRIRDAVIKLSSKIPEVGTAVGILVGFFWPQDKVDIFESIKADITKLVKKEILDYELSAHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 98 - protein, 445 aa, synthetic construct
MADLVASNWNNRIRDAVIKLSSKIPEVGTAVGILVGFFWPQDKVDIFESIKADITKLVKKEILDYELSLAKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 99 - protein, 445 aa, synthetic construct
MADLVASNWNNRIRDAVIKLSSKIPEVGTAVGILVGFFWPQDKVDIFESIKADITKLVKKEILDYELSLHANELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 100 - protein, 445 aa, synthetic construct
MADLVASNWNNRIRDAVIKLSSKIPEVGTAVGILVGFFWPQDKVDIFESIKADITKLVKKEILDYELSLHKAELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 101 - protein, 445 aa, synthetic construct
MADLVASNWNNRIRDAVIKLSSKIPEVGTAVGILVGFFWPQDKVDIFESIKADITKLVKKEILDYELSLHKNALDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 102 - protein, 445 aa, synthetic construct
MADLVASNWNNRIRDAVIKLSSKIPEVGTAVGILVGFFWPQDKVDIFESIKADITKLVKKEILDYELSLHKNEADALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 103 - protein, 445 aa, synthetic construct
MADLVASNWNNRIRDAVIKLSSKIPEVGTAVGILVGFFWPQDKVDIFESIKADITKLVKKEILDYELSLHKNELAALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 104 - protein, 445 aa, synthetic construct
MADLVASNWNNRIRDAVIKLSSKIPEVGTAVGILVGFFWPQDKVDIFESIKADITKLVKKEILDYELSLHKNELDSLKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 105 - protein, 445 aa, synthetic construct
MADLVASNWNNRIRDAVIKLSSKIPEVGTAVGILVGFFWPQDKVDIFESIKADITKLVKKEILDYELSLHKNELDAAKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 106 - protein, 445 aa, synthetic construct
MADLVASNWNNRIRDAVIKLSSKIPEVGTAVGILVGFFWPQDKVDIFESIKADITKLVKKEILDYELSLHKNELDALAETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 107 - protein, 445 aa, synthetic construct
MADLVASNWNNRIRDAVIKLSSKIPEVGTAVGILVGFFWPQDKVDIFESIKADITKLVKKEILDYELSLHKNELDALKATMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 108 - protein, 445 aa, synthetic construct
MADLVASNWNNRIRDAVIKLSSKIPEVGTAVGILVGFFWPQDKVDIFESIKADITKLVKKEILDYELSLHKNELDALKEAMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 109 - protein, 445 aa, synthetic construct
MADLVASNWNNRIRDAVIKLSSKIPEVGTAVGILVGFFWPQDKVDIFESIKADITKLVKKEILDYELSLHKNELDALKETAKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 110 - protein, 445 aa, synthetic construct
MADLVASNWNNRIRDAVIKLSSKIPEVGTAVGILVGFFWPQDKVDIFESIKADITKLVKKEILDYELSLHKNELDALKETMARYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 111 - protein, 445 aa, synthetic construct
MADLVASNWNNRIRDAVIKLSSKIPEVGTAVGILVGFFWPQDKVDIFESIKADITKLVKKEILDYELSLHKNELDALKETMKAYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 112 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRAETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 113 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYATAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 114 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYEAAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 115 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETSMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 116 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAANHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 117 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMAHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 118 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNAEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 119 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHAKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 120 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEAGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 121 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKALFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 122 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGAFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 123 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLALNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 124 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFANSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 125 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLASWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 126 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNAWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 127 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSAINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 128 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWANEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 129 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWIAEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 130 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINAADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 131 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINESDRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 132 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEAARLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 133 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADALSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 134 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRASVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 135 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLAVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 136 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSAIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 137 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVAFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 138 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIARQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 139 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFAQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 140 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRASANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 141 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQAANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 142 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSSNNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 143 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSAANIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 144 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANAIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 145 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNAHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 146 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIALIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 147 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHAIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 148 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLAQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 149 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIALVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 150 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQAVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 151 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLAISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 152 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVASLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 153 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVIALALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 154 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISAALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 155 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLSLLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 156 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLAALHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 157 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALAHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 158 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLALTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 159 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHATALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 160 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLAALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 161 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTSLRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 162 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTAARERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 163 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALAERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 164 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRARFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 165 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALREAFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 166 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERADFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 167 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFAFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 168 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDAGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 169 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFARSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 170 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGASLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 171 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRALYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 172 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSAYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 173 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLASEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 174 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYAEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 175 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSADNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 176 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEANTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 177 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDATPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 178 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNAPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 179 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTAQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 180 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPAWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 181 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQAKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 182 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWAEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 183 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKADLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 184 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEALENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 185 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDAENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 186 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLANMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 187 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLEAMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 188 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENAYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 189 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMAKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 190 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYATYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 191 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKAYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 192 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTAIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 193 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYAVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 194 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIADFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 195 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVAFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 196 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDAIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 197 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFAPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 198 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIADIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 199 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPAIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 200 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDAYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 201 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIAKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 202 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYAKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 203 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKAWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 204 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKAKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 205 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWAEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 206 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKAWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 207 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEARTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 208 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWATNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 209 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRANQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 210 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTAQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 211 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNAVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 212 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQAEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 213 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVAISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 214 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEASSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 215 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEIASWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 216 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISAWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 217 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSALTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 218 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWATQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 219 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLAQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 220 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTARPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 221 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQAPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 222 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRAITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 223 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPATLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 224 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPIALPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 225 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITAPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 226 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLAPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 227 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPAFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 228 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPAYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 229 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFATNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 230 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYANESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 231 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTAESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 232 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNASHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 233 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNEAHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 234 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESAARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 235 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHSRVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 236 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHAAVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 237 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARAVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 238 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVADNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 239 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVANVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 240 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDAVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 241 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNATGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 242 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVAGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 243 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTAEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 244 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGAIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 245 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEAKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 246 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIANFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 247 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKAFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 248 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNAYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 249 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFAQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 250 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYADLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 251 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQALSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 252 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDASSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 253 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLASSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 254 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSASTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 255 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSATSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 256 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSASTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 257 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTATFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 258 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSAFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 259 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTANNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 260 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFANICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 261 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNAICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 262 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNACQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 263 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNIAQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 264 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICAAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 265 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQSHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 266 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAAKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 267 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHATRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 268 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKARMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 269 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTAMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 270 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRACNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 271 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMANDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 272 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCADAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 273 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNAAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 274 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDSYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 275 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAACDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 276 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYADMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 277 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCAMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 278 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDAVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 279 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMAGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 280 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVASISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 281 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGAISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 282 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSASATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 283 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSIAATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 284 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISSTFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 285 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISAAFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 286 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATASYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 287 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFAYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 288 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSALQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 289 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYAQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 290 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLAILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 291 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQALPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 292 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQIAPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 293 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILADNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 294 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPANNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 295 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDANKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 296 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNAKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 297 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNANFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 298 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKAFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 299 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNAAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 300 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFSKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 301 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAAYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 302 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKADKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 303 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYAKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 304 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDAEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 305 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKAVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 306 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEAFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 307 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVAGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 308 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFAEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 309 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGAVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 310 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEAFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 311 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVAKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 312 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFAGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 313 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKAPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 314 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGAYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 315 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPASLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 316 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYALDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 317 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSADLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 318 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLALSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 319 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDASYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 320 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLAYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 321 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSASNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 322 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYANYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 323 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSAYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 324 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNASYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 325 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYAYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 326 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSAGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 327 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYAYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 328 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGAGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 329 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYANNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 330 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGANVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 331 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNAVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 332 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNAPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 333 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVASFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 334 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPAFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 335 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSARINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 336 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFAINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 337 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRANPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 338 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRIAPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 339 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINATNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 340 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPANNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 341 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTANDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 342 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNADYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 343 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNAYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 344 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDASSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 345 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYASSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 346 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSASAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 347 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSAAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 348 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSSGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 349 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAAPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 350 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGAITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 351 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPATKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 352 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPIAKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 353 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITAVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 354 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKAIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 355 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVAIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 356 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIAREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 357 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIAEWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 358 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIRAWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 359 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREANSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 360 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWASIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 361 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNAIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 362 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSADAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 363 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIAAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 364 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDSMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 365 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAAQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 366 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMAFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 367 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQAIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 368 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFAYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 369 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIAQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 370 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYAGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 371 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQARNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 372 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGANGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 373 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRAGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 374 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNASMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 375 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGAMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 376 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSAAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 377 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMSGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 378 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAANPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 379 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGAPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 380 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNASGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 381 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPAGGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 382 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSAGLQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 383 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGALQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 384 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGAQHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 385 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLAHDIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 386 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQADIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 387 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHAIDVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 388 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDADVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 389 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIAVSNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 390 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDASNKLIHGFHLGFSSGVLARIKVDYYDGTSSDMLGNRGGWPITEQVAKGPTLYKVISWSYKVDYGPSGTYGPSVIQLQYTPHMD

SEQ ID NO: 391 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVANKLIHGFHLGFSSGVLARIKVDYYDGTSSDMLGNRGGWPITEQVAKGPTLYKVISWSYKVDYGPSGTYGPSVIQLQYTPHMD

SEQ ID NO: 392 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSAKLIHGFHLGFSSGVLARIKVDYYDGTSSDMLGNRGGWPITEQVAKGPTLYKVISWSYKVDYGPSGTYGPSVIQLQYTPHMD

SEQ ID NO: 393 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNALIHGFHLGFSSGVLARIKVDYYDGTSSDMLGNRGGWPITEQVAKGPTLYKVISWSYKVDYGPSGTYGPSVIQLQYTPHMD

SEQ ID NO: 394 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKAIHGFHLGFSSGVLARIKVDYYDGTSSDMLGNRGGWPITEQVAKGPTLYKVISWSYKVDYGPSGTYGPSVIQLQYTPHMD

SEQ ID NO: 395 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLAHGFHLGFSSGVLARIKVDYYDGTSSDMLGNRGGWPITEQVAKGPTLYKVISWSYKVDYGPSGTYGPSVIQLQYTPHMD

SEQ ID NO: 396 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIAGFHLGFSSGVLARIKVDYYDGTSSDMLGNRGGWPITEQVAKGPTLYKVISWSYKVDYGPSGTYGPSVIQLQYTPHMD

SEQ ID NO: 397 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHAFHLGFSSGVLARIKVDYYDGTSSDMLGNRGGWPITEQVAKGPTLYKVISWSYKVDYGPSGTYGPSVIQLQYTPHMD

SEQ ID NO: 398 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGAHLGFSSGVLARIKVDYYDGTSSDMLGNRGGWPITEQVAKGPTLYKVISWSYKVDYGPSGTYGPSVIQLQYTPHMD

SEQ ID NO: 399 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFALGFSSGVLARIKVDYYDGTSSDMLGNRGGWPITEQVAKGPTLYKVISWSYKVDYGPSGTYGPSVIQLQYTPHMD

SEQ ID NO: 400 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHAGFSSGVLARIKVDYYDGTSSDMLGNRGGWPITEQVAKGPTLYKVISWSYKVDYGPSGTYGPSVIQLQYTPHMD

SEQ ID NO: 401 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLAFSSGVLARIKVDYYDGTSSDMLGNRGGWPITEQVAKGPTLYKVISWSYKVDYGPSGTYGPSVIQLQYTPHMD

SEQ ID NO: 402 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGASSGVLARIKVDYYDGTSSDMLGNRGGWPITEQVAKGPTLYKVISWSYKVDYGPSGTYGPSVIQLQYTPHMD

SEQ ID NO: 403 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFASGVLARIKVDYYDGTSSDMLGNRGGWPITEQVAKGPTLYKVISWSYKVDYGPSGTYGPSVIQLQYTPHMD

SEQ ID NO: 404 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSAGVLARIKVDYYDGTSSDMLGNRGGWPITEQVAKGPTLYKVISWSYKVDYGPSGTYGPSVIQLQYTPHMD

SEQ ID NO: 405 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSAVLARIKVDYYDGTSSDMLGNRGGWPITEQVAKGPTLYKVISWSYKVDYGPSGTYGPSVIQLQYTPHMD

SEQ ID NO: 406 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGALARIKVDYYDGTSSDMLGNRGGWPITEQVAKGPTLYKVISWSYKVDYGPSGTYGPSVIQLQYTPHMD

SEQ ID NO: 407 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVAARIKVDYYDGTSSDMLGNRGGWPITEQVAKGPTLYKVISWSYKVDYGPSGTYGPSVIQLQYTPHMD

SEQ ID NO: 408 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLSRIKVDYYDGTSSDMLGNRGGWPITEQVAKGPTLYKVISWSYKVDYGPSGTYGPSVIQLQYTPHMD

SEQ ID NO: 409 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLAAIKVDYYDGTSSDMLGNRGGWPITEQVAKGPTLYKVISWSYKVDYGPSGTYGPSVIQLQYTPHMD

SEQ ID NO: 410 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARAKVDYYDGTSSDMLGNRGGWPITEQVAKGPTLYKVISWSYKVDYGPSGTYGPSVIQLQYTPHMD

SEQ ID NO: 411 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIAVDYYDGTSSDMLGNRGGWPITEQVAKGPTLYKVISWSYKVDYGPSGTYGPSVIQLQYTPHMD

SEQ ID NO: 412 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKADYYDGTSSDMLGNRGGWPITEQVAKGPTLYKVISWSYKVDYGPSGTYGPSVIQLQYTPHMD

SEQ ID NO: 413 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVAYYDGTSSDMLGNRGGWPITEQVAKGPTLYKVISWSYKVDYGPSGTYGPSVIQLQYTPHMD

SEQ ID NO: 414 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDAYDGTSSDMLGNRGGWPITEQVAKGPTLYKVISWSYKVDYGPSGTYGPSVIQLQYTPHMD

SEQ ID NO: 415 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYADGTSSDMLGNRGGWPITEQVAKGPTLYKVISWSYKVDYGPSGTYGPSVIQLQYTPHMD

SEQ ID NO: 416 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYAGTSSDMLGNRGGWPITEQVAKGPTLYKVISWSYKVDYGPSGTYGPSVIQLQYTPHMD

SEQ ID NO: 417 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDATSSDMLGNRGGWPITEQVAKGPTLYKVISWSYKVDYGPSGTYGPSVIQLQYTPHMD

SEQ ID NO: 418 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGASSDMLGNRGGWPITEQVAKGPTLYKVISWSYKVDYGPSGTYGPSVIQLQYTPHMD

SEQ ID NO: 419 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTASDMLGNRGGWPITEQVAKGPTLYKVISWSYKVDYGPSGTYGPSVIQLQYTPHMD

SEQ ID NO: 420 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSADMLGNRGGWPITEQVAKGPTLYKVISWSYKVDYGPSGTYGPSVIQLQYTPHMD

SEQ ID NO: 421 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSAMLGNRGGWPITEQVAKGPTLYKVISWSYKVDYGPSGTYGPSVIQLQYTPHMD

SEQ ID NO: 422 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDALGNRGGWPITEQVAKGPTLYKVISWSYKVDYGPSGTYGPSVIQLQYTPHMD

SEQ ID NO: 423 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMAGNRGGWPITEQVAKGPTLYKVISWSYKVDYGPSGTYGPSVIQLQYTPHMD

SEQ ID NO: 424 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLANRGGWPITEQVAKGPTLYKVISWSYKVDYGPSGTYGPSVIQLQYTPHMD

SEQ ID NO: 425 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGARGGWPITEQVAKGPTLYKVISWSYKVDYGPSGTYGPSVIQLQYTPHMD

SEQ ID NO: 426 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNAGGWPITEQVAKGPTLYKVISWSYKVDYGPSGTYGPSVIQLQYTPHMD

SEQ ID NO: 427 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRAGWPITEQVAKGPTLYKVISWSYKVDYGPSGTYGPSVIQLQYTPHMD

SEQ ID NO: 428 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGAWPITEQVAKGPTLYKVISWSYKVDYGPSGTYGPSVIQLQYTPHMD

SEQ ID NO: 429 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGAPITEQVAKGPTLYKVISWSYKVDYGPSGTYGPSVIQLQYTPHMD

SEQ ID NO: 430 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWAITEQVAKGPTLYKVISWSYKVDYGPSGTYGPSVIQLQYTPHMD

SEQ ID NO: 431 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPATEQVAKGPTLYKVISWSYKVDYGPSGTYGPSVIQLQYTPHMD

SEQ ID NO: 432 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPIAEQVAKGPTLYKVISWSYKVDYGPSGTYGPSVIQLQYTPHMD

SEQ ID NO: 433 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITAQVAKGPTLYKVISWSYKVDYGPSGTYGPSVIQLQYTPHMD

SEQ ID NO: 434 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEAVAKGPTLYKVISWSYKVDYGPSGTYGPSVIQLQYTPHMD

SEQ ID NO: 435 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQAAKGPTLYKVISWSYKVDYGPSGTYGPSVIQLQYTPHMD

SEQ ID NO: 436 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVSKGPTLYKVISWSYKVDYGPSGTYGPSVIQLQYTPHMD

SEQ ID NO: 437 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAAGPTLYKVISWSYKVDYGPSGTYGPSVIQLQYTPHMD

SEQ ID NO: 438 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKAPTLYKVISWSYKVDYGPSGTYGPSVIQLQYTPHMD

SEQ ID NO: 439 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGATLYKVISWSYKVDYGPSGTYGPSVIQLQYTPHMD

SEQ ID NO: 440 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPALYKVISWSYKVDYGPSGTYGPSVIQLQYTPHMD

SEQ ID NO: 441 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTAYKVISWSYKVDYGPSGTYGPSVIQLQYTPHMD

SEQ ID NO: 442 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLAKVISWSYKVDYGPSGTYGPSVIQLQYTPHMD

SEQ ID NO: 443 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYAVISWSYKVDYGPSGTYGPSVIQLQYTPHMD

SEQ ID NO: 444 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKAISWSYKVDYGPSGTYGPSVIQLQYTPHMD

SEQ ID NO: 445 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVASWSYKVDYGPSGTYGPSVIQLQYTPHMD

SEQ ID NO: 446 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVIAWSYKVDYGPSGTYGPSVIQLQYTPHMD

SEQ ID NO: 447 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISASYKVDYGPSGTYGPSVIQLQYTPHMD

SEQ ID NO: 448 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWAYKVDYGPSGTYGPSVIQLQYTPHMD

SEQ ID NO: 449 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSAKVDYGPSGTYGPSVIQLQYTPHMD

SEQ ID NO: 450 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYAVDYGPSGTYGPSVIQLQYTPHMD

SEQ ID NO: 451 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKADYGPSGTYGPSVIQLQYTPHMD

SEQ ID NO: 452 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVAYGPSGTYGPSVIQLQYTPHMD

SEQ ID NO: 453 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDAGPSGTYGPSVIQLQYTPHMD

SEQ ID NO: 454 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYAPSGTYGPSVIQLQYTPHMD

SEQ ID NO: 455 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGASGTYGPSVIQLQYTPHMD

SEQ ID NO: 456 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPAGTYGPSVIQLQYTPHMD

SEQ ID NO: 457 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSATYGPSVIQLQYTPHMD

SEQ ID NO: 458 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGAYGPSVIQLQYTPHMD

SEQ ID NO: 459 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTAGPSVIQLQYTPHMD

SEQ ID NO: 460 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYAPSVIQLQYTPHMD

SEQ ID NO: 461 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGASVIQLQYTPHMD

SEQ ID NO: 462 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPAVIQLQYTPHMD

SEQ ID NO: 463 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSAIQLQYTPHMD

SEQ ID NO: 464 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVAQLQYTPHMD

SEQ ID NO: 465 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIALQYTPHMD

SEQ ID NO: 466 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQAQYTPHMD

SEQ ID NO: 467 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLAYTPHMD

SEQ ID NO: 468 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQATPHMD

SEQ ID NO: 469 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYAPHMD

SEQ ID NO: 470 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTAHMD

SEQ ID NO: 471 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPAMD

SEQ ID NO: 472 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHAD

SEQ ID NO: 473 - protein, 445 aa, synthetic construct
MADLVASNWNNRIRDAVIKLSSKIPEVGTAVGILVGFFWPQDKVDIFESIKADITKLVKKEILDYELSLHKNELDALKETMKRYETAMNHEKGLFLNSWINEADRLSVIFRQSANNIHLIQLVISLALLHLTALRERFDFGRSLYSEDNTPQWKEDLENMYKTYIVDFIPDIYKKWKEWRTNQVEISSWLTQRPITLPPFYTNESHARVVDNVTGEIKNFYQDLSSSTSTFNNICQAHKTRMCNDAYCDMVGSISATFSYLQILPDNNKNFAKYDKEVFGEVFKGPYSLDLSYSNYSYGYGNNVPSFRINPTNNDYSSSAGPITKVIIREWNSIDAMQFIYQGRNGSMAGNPSGGLQHDIDVSNKLIHGFHLGFSSGVLARIKVDYYDGTSSDMLGNRGGWPITEQVAKGPTLYKVISWSYKVDYGPSGTYGPSVIQLQYTPHMA

(19) United States
(12) Patent Application Publication
(10) Pub. No.: US 2026/0344676 A1
(43) Pub. Date: Nov. 5, 2026

(54) Compositions and Methods For Controlling Coleopteran Pests

(71) Applicant: Syngenta Crop Protection AG, Basel (CH)

(72) Inventors: Clarence Michael Reynolds, Research Triangle Park, NC (US); Jorge Duarte, Saint-Sauveur (FR)

(73) Assignee: Syngenta Crop Protection AG, Basel (CH)

(21) Appl. No.: 19/302,628

(22) Filed: Aug. 18, 2025

Related U.S. Application Data
(60) Provisional application No. 63/685,039, filed on Aug. 20, 2024.

(51) Int. Cl. A01N 63/50; A01P 7/04; C07K 14/415; C12N 15/82

(52) U.S. Cl. CPC A01N 63/50; A01P 7/04; C07K 14/415; C12N 15/8286

(57) ABSTRACT

Novel insecticidal polypeptides that are active against Coleopteran pests are disclosed. Nucleic acid molecules encoding the novel insecticidal proteins are also provided. The nucleotide sequences encoding the insecticidal polypeptides can be used to transform prokaryotic and eukaryotic organisms to express the insecticidal proteins. Methods of making the insecticidal proteins and methods of using the insecticidal proteins, for example in transgenic plants to confer protection from insect damage are also disclosed.

### RELATED APPLICATION INFORMATION

This application claims priority to U.S. Application No. 63/685,039, filed on Aug. 20, 2024, the contents of which are incorporated by reference herein.

### STATEMENT REGARDING ELECTRONIC FILING OF A SEQUENCE LISTING

A Sequence Listing in XML format, submitted under 37 C.F.R. § 1.831(a), entitled “83329conversionsequencelisting_25July25.xml”, approximately 626,825 bytes in size, generated on Jul. 25, 2025, and filed via EFS-Web, is provided in lieu of a paper copy. This Sequence Listing is hereby incorporated herein by reference into the specification for its disclosures.

### FIELD OF THE INVENTION

The present disclosure relates to novel pesticidal proteins having insecticidal activity, nucleic acid molecules that encode for and whose expression results in the pesticidal proteins, as well as compositions and methods for controlling agriculturally relevant pests of crop plants.

### BACKGROUND

Insects are a major cause of crop losses. Numerous commercially valuable plants, including common agricultural crops, are susceptible to attack by plant pests including insects and nematodes, causing substantial reductions in crop yield and quality. For example, plant pests are a major factor in the loss of the world's important agricultural crops. Insect pests are also a burden to vegetable and fruit growers, to producers of ornamental flowers, and they are a nuisance to home gardeners.

Species of corn rootworm are considered to be the most destructive corn pests. In the United States, the three important species are Diabrotica virgifera virgifera, the western corn rootworm, D. longicornis barberi, the northern corn rootworm and D. undecimpunctata howardi, the southern corn rootworm. Only western and northern corn rootworms are considered primary pests of corn in the US Corn Belt. Additionally, an important corn rootworm pest in the Southern US is the Mexican corn rootworm, Diabrotica virgifera zeae. Corn rootworm larvae cause substantial plant damage by feeding almost exclusively on corn roots. This injury has been shown to increase plant lodging, to reduce grain yield and vegetative yield as well as alter the nutrient content of the grain. Larval feeding also causes indirect effects on corn by opening avenues through the roots for bacterial and fungal infections potentially leading to root and stalk rot diseases. Adult corn rootworms are active in cornfields in late summer where they feed on ears, silks and pollen, thus interfering with normal pollination.

Corn rootworms are mainly controlled by intensive applications of chemical pesticides, which are active through inhibition of insect growth, prevention of insect feeding or reproduction, or death. Good corn rootworm control can thus be reached but not without some inefficiencies. In some cases, application of these chemicals can affect other beneficial organisms. Additionally, the wide use of chemical pesticides can result in the development of resistant insect varieties. Lastly, the underground feeding preferences of corn rootworm larvae can make it difficult to apply rescue treatments of insecticides. Therefore, most insecticide applications are made prophylactically at the time of planting which results in a large environmental burden. This has been partially alleviated by various farm management practices, but there is an increasing need for alternative pest control mechanisms.

Biological pest control agents, such as Bacillus thuringiensis (Bt) strains expressing pesticidal toxins like δ-endotoxins (delta-endotoxins; also called crystal toxins or Cry proteins), have been applied to crop plants with satisfactory results against insect pests. The δ-endotoxins are proteins held within a crystalline matrix that are known to possess insecticidal activity when ingested by certain orders and species of plant pests, including insects, but are harmless to plants and other non-target organisms. Several native Cry proteins from Bacillus thuringiensis, or engineered Cry proteins, have been expressed in transgenic crop plants to control certain Lepidopteran and Coleopteran insect pests as an alternative to or complement to chemical pesticides. Transgenic corn hybrids that control corn rootworm have been available commercially in the US since 2003 and express toxins such as Cry3Bb1, Cry34Ab1/Cry35Ab1, modified Cry3A (mCry3A), or Cry3Ab (eCry3.1Ab).

Although the usage of transgenic plants expressing Cry proteins has been shown to be extremely effective, insect pests that now have resistance against the Cry proteins expressed in certain transgenic plants are known. Therefore, there remains a need to identify new and effective pest control agents that provide an economic benefit to farmers and that are environmentally acceptable. Particularly needed are proteins that are toxic to Diabrotica species, a major pest of corn, that have a different mode of action than existing insect control products as a way to mitigate the development of resistance. Furthermore, delivery of insect control agents through products that minimize the burden on the environment, as through transgenic plants, are desirable.

### SUMMARY

This disclosure provides polypeptides that are insecticidal against at least a coleopteran pest, e.g., against corn rootworm (WCR, Diabrotica virgifera virgifera) and uses of such polypeptides and related nucleic acids in compositions and methods, such as in plants or in methods of controlling a coleopteran pest.

Accordingly in some aspects, there is provided a polypeptide comprising an amino acid sequence that is at least 70% identical to any one of SEQ ID NOs: 1-4. In some embodiments, the polypeptide is at least 70% (e.g., at least at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%) identical to any one of SEQ ID NOs: 1-4. In some embodiments, the polypeptide is at least 90% (e.g., at least 90%, at least 91%, at least 92%, at least 93%, at least 94, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%) identical to any one of SEQ ID NOs: 1-4. Further aspects of the disclosure include the polypeptide that comprises any one of SEQ ID NOs: 1-4. In some embodiments, the polypeptide is insecticidal against a coleopteran pest. In some embodiments, the polypeptide is insecticidal against a Diabrotica pest (e.g., Diabrotica virgifera virgifera).

Also provided by the present disclosure is a polypeptide comprising an amino acid sequence that is at least 95% (e.g., at least 95%, 96%, 97%, 98% or 99%) identical to SEQ ID NO:1 and that is toxic to a coleopteran pest, e.g., a Diabrotica pest such as Western corn rootworm (Diabrotica virgifera virgifera). The disclosure also encompasses a polypeptide comprising an amino acid sequence that is at least 95% identical to SEQ ID NO:1 and that is toxic to a coleopteran pest, e.g., a Diabrotica pest such as Western corn rootworm (Diabrotica virgifera virgifera) wherein the polypeptide is derived from a Marsilea fern. Further aspects of the disclosure include a polypeptide consisting of the amino acid sequence of SEQ ID NO: 1 or differing from the amino acid sequence of SEQ ID NO: 1 by conservative substitution, deletion, and/or addition of 1-5 amino acids, wherein the polypeptide is toxic to a coleopteran pest, e.g., a Diabrotica pest such as Western corn rootworm. In some embodiments, the polypeptide consists of any one of SEQ ID NOs: 1-4.

A further aspect of the present disclosure is a nucleic acid molecule comprising a coding sequence that encodes the polypeptide of any of the above-mentioned embodiments. In some embodiments, the coding sequence comprises a nucleotide sequence that is at least 80% identical (e.g., at least 80%, 85%, 90%, 95%, 96%, 97%, 98%, or 99%) to or comprises any one of SEQ ID NOs: 5-9. In some embodiments, the coding sequence is codon optimized for expression in a plant (e.g., a maize plant). In some embodiments, the coding sequence is operably linked to a heterologous promoter, e.g., a plant-expressible heterologous promoter.

A still further aspect of the present disclosure are vectors comprising any of the described nucleic acid molecules and transgenic host cells comprising any of the described polypeptides or any of the described nucleic acid molecules. In some embodiments, the transgenic host cell is a plant cell, such as a monocot cell, e.g., a maize cell. In some embodiments, the transgenic host cell is a bacterial cell, such as an Agrobacterium, Bacillus, or an Escherichia coli cell.

Additional aspects of the present disclosure are a composition comprising any of the described polypeptides, optionally further comprising an agriculturally acceptable carrier. A still further aspect of the present disclosure are plants comprising any of the described polypeptides or comprising any of the described nucleic acid molecules. The present disclosure contemplates that these plants are monocots, and these plants are maize plants. A further aspect of the presentation disclosure are seeds of any of the described plants and cells of any of the described plants.

The present disclosure also provides a method of producing a transgenic plant, the method comprising the steps of introducing into a plant cell any of the described nucleic acid molecules, selecting a plant cell comprising the nucleic acid molecule; and regenerating a plant from the selected plant cell. Also provided is a method for producing a transgenic plant comprising the steps of sexually crossing a first parent plant with a second parent plant, where the first or second parent plant comprises a polypeptide or nucleic acid molecule of the disclosure and selecting a progeny plant with enhanced insecticidal properties, wherein the selected progeny plant comprises the nucleic acid molecule. In some embodiments, the method further comprises selfing the progeny plant, thereby producing a plurality of second generation progeny plants; and selecting from the second generation progeny plants a plant with enhanced insecticidal properties, wherein the selected second generation progeny plants comprise the nucleic acid molecule.

The disclosure also provides a method of identifying a candidate insecticidal toxin, the method comprising the steps of providing a three-dimensional structure of a polypeptide comprising any one of SEQ ID NOs: 1 to 4, identifying active regions or positions within the polypeptide amino acid sequence that contribute to insecticidal activity, computationally generating a plurality of putative toxin amino acid sequences, wherein the active regions or positions are maintained and at least one other portion of the polypeptide amino acid sequence is modified to include at least one modification relative to the polypeptide amino acid sequence, predicting three-dimensional structures for the plurality of putative toxin sequences, evaluating whether the three-dimensional structures of the plurality of putative toxin sequences, optionally by evaluating structural quality, energy minimization, and/or predicted insecticidal activity; and selecting candidate insecticidal toxins based on the evaluation.

The disclosure is also related to methods of controlling a coleopteran pest comprising delivering to the pest or an environment thereof any of the described polypeptides, e.g., in a plant or composition. In some embodiments, the coleopteran pest is a Diabrotica pest such as Western corn rootworm (D. virgifera virgifera). In some embodiments, the polypeptide is delivered by feeding, e.g., the pest feeding on a plant part that comprises the described polypeptide. The present disclosure also contemplates the use of the sequence of any of SEQ ID NOs: 1 to 9 in a bioinformatic analysis to identify an insecticidal protein and the use of a polypeptide comprising any of the amino acid sequences of any one of SEQ ID NOs: 1-4 in an insect bioassay to identify an insecticidal protein (e.g. insecticidal against a coleopteran pest, e.g., a Diabrotica pest such as Western corn rootworm (D. virgifera virgifera)).

### BRIEF DESCRIPTION OF THE SEQUENCES IN THE SEQUENCE LISTING

SEQ ID NO:1 is the amino acid sequence of Mv6_CRW_v1 identified in Marsilea vestita
SEQ ID NO:2 is the amino acid sequence of Mv6_CRW_v2 identified in Marsilea vestita
SEQ ID NO: 3 is the amino acid sequence of Mv6_CRW_v3 identified in Marsilea vestita
SEQ ID NO: 4 is the amino acid sequence of Mv6_CRW_v4 identified in Marsilea vestita
SEQ ID NO: 5 is the native nucleotide sequence of Mv6_CRW_v1
SEQ ID NO: 6 is the native nucleotide sequence of Mv6_CRW_v2
SEQ ID NO: 7 is the native nucleotide sequence of Mv6_CRW_v3
SEQ ID NO: 8 is the native nucleotide sequence of Mv6_CRW_v4
SEQ ID NO: 9 is an E. coli optimized nucleotide sequence of Mv6_CRW_v1
SEQ ID NO: 10 is an E. coli optimized nucleotide sequence of Mv6_CRW_v2
SEQ ID NO: 11 is an E. coli optimized nucleotide sequence of Mv6_CRW_v3
SEQ ID NO: 12 is an E. coli optimized nucleotide sequence of Mv6_CRW_v4
SEQ ID NO: 13 is a maize optimized nucleotide sequence of Mv6_CRW_v1
SEQ ID NO: 14 is a maize optimized nucleotide sequence of Mv6_CRW_v2
SEQ ID NO: 15 is a maize optimized nucleotide sequence of Mv6_CRW_v3
SEQ ID NO: 16 is a maize optimized nucleotide sequence of Mv6_CRW_v4
SEQ ID NO: 17 is an N-terminal 6 Histidine+5 residue linker used in protein purification
SEQ ID NO: 18 is the nucleotide sequence of OTPSSUct, a chimeric Sunflower SSU and Maize SSU plastid optimized transit peptide
SEQ ID NO: 19 is the amino acid sequence of OTPSSUct.
SEQ ID NO: 20 is the nucleotide sequence of OsCTP6, a chloroplast transit peptide of the rice Os01t0802100 gene, which encodes a GHMP kinase.
SEQ ID NO: 21 is the amino acid sequence of OsCTP6.
SEQ ID NO: 22 is the nucleotide sequence of AtCTP2, an Arabidopsis thalania EPSP synthase chloroplast transit peptide.
SEQ ID NO: 23 is the amino acid sequence of AtCTP2
SEQ ID NO: 24 is the nucleotide sequence of SbCTP15, a transit peptide of the sorghum Sobic.004G202200 gene.
SEQ ID NO:25 is the amino acid sequence of SbCTP15.
SEQ ID NO: 26 is the amino acid sequence of the transit peptide OTPssUct fused with Mv6_CRW_v1.
SEQ ID NO: 27 is the amino acid sequence of the transit peptide OsCTP6 fused with Mv6_CRW_v1.
SEQ ID NO: 28 is the amino acid sequence of the transit peptide AtCTP2 fused with Mv6_CRW_v1.
SEQ ID NO: 29 is the amino acid sequence of the transit peptide AbCTP15 fused with Mv6_CRW_v1.
SEQ ID NOs: 30-473 are amino acid sequences of the Mv6_CRW_v1 alanine scanning variants.

### DETAILED DESCRIPTION

This description is not intended to be a detailed catalog of all the different ways in which the disclosure may be implemented, or all the features that may be added to the instant disclosure. For example, features illustrated with respect to one embodiment may be incorporated into other embodiments, and features illustrated with respect to a particular embodiment may be deleted from that embodiment. Thus, the disclosure contemplates that in some embodiments, any feature or combination of features set forth herein can be excluded or omitted. In addition, numerous variations and additions to the various embodiments suggested herein will be apparent to those skilled in the art in light of the instant disclosure, which do not depart from the instant disclosure. Hence, the following descriptions are intended to illustrate some particular embodiments of the disclosure, and not to exhaustively specify all permutations, combinations, and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

All publications, patent applications, patents and other references cited herein are incorporated by reference in their entireties for the teachings relevant to the sentence and/or paragraph in which the reference is presented.

Nucleotide sequences provided herein are presented in the 5′ to 3′ direction, from left to right and are presented using the standard code for representing nucleotide bases as set forth in 37 CFR §§ 1.821-1.825 and the World Intellectual Property Organization (WIPO) Standard ST.25, for example: adenine (A), cytosine (C), thymine (T), and guanine (G).

Amino acids are likewise indicated using the WIPO Standard ST.25, for example: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; 1), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

### DEFINITIONS

For clarity, certain terms used in the specification are defined and presented as follows:

As used herein and in the appended claims, the singular forms “a,” “an,” and “the” include plural reference unless the context clearly dictates otherwise. Thus, for example, reference to “a plant” is a reference to one or more plants and includes equivalents thereof known to those skilled in the art, and so forth.

As used herein, the word “or” also encompasses “and/or” unless the context clearly indicates otherwise.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means ±1 °C, preferably ±0.5 °C. Where the term “about” is used in the context of this disclosure (e.g., in combinations with temperature or molecular weight values) the exact value (i.e., without “about”) is preferred.

As used herein, phrases such as “between about X and Y”, “between about X and about Y”, “from X to Y” and “from about X to about Y” (and similar phrases) should be interpreted to include X and Y, unless the context indicates otherwise. Units, prefixes and symbols may be denoted in their SI accepted form. Unless otherwise indicated, nucleic acids are written left to right in 5′ to 3′ orientation; amino acid sequences are written left to right in N-terminus to C-terminus orientation, respectively. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Activity” of the insecticidal proteins of the disclosure is meant that the insecticidal proteins function as orally active insect control agents, have a toxic effect and thereby control a pest population (e.g., inhibiting and/or decreasing the ability of the insect pest to survive, grow, and/or reproduce), and/or are able to disrupt or deter insect feeding, which may or may not cause death of the insect. When an insecticidal protein of the disclosure is delivered to the insect, the result is typically death of the insect, or the insect does not feed upon the source that makes the insecticidal protein available to the insect. “Pesticidal” is defined as a toxic biological activity capable of controlling a pest, such as an insect, nematode, fungus, bacteria, or virus, preferably by killing or destroying them. “Insecticidal” is defined as a toxic biological activity capable of controlling insects, preferably by killing them. A “pesticidal agent” is an agent that has pesticidal activity. An “insecticidal agent” is an agent that has insecticidal activity.

A “coding sequence” is a nucleic acid sequence that is transcribed into RNA such as mRNA, rRNA, tRNA, snRNA, sense RNA or antisense RNA which is then preferably translated in an organism to produce a protein.

As used herein, a “codon optimized” sequence means a nucleotide sequence wherein the codons are chosen to reflect the particular codon bias that a host cell or organism may have. This is typically done in such a way so as to preserve the amino acid sequence of the polypeptide encoded by the nucleotide sequence to be optimized. In certain embodiments, the DNA sequence of the recombinant DNA construct includes sequence that has been codon optimized for the cell (e.g., an animal, plant, or fungal cell) in which the construct is to be expressed. For example, a construct to be expressed in a plant cell can have all or parts of its sequence (e.g., the first gene suppression element or the gene expression element) codon optimized for expression in a plant. See, for example, U.S. Pat. No. 6,121,014, which is incorporated herein by reference. In some embodiments, the polynucleotides of the disclosure are codon-optimized for expression in a plant cell (e.g., a dicot cell or a monocot cell) or bacterial cell.

To “control” insects means to inhibit, through a toxic effect, the ability of insect pests to survive, grow, feed, and/or reproduce, and/or to limit insect-related damage or loss in crop plants and/or to protect the yield potential of a crop when grown in the presence of insect pests. To “control” insects may or may not mean killing the insects, although it preferably means killing the insects. In some embodiments of the disclosure, “control” of the insect means killing the insects.

The terms “comprises”, “comprising”, “includes”, “including”, “having” and their conjugates mean including “but not limited to”. These terms specify the presence of stated features, integers, steps, operations, elements, or components, but do not preclude the presence or addition of one or more other features, integers, steps, operations, elements, components, or groups thereof. The term “consisting of means “including and limited to”.

As used herein, the transitional phrase “consisting essentially of” (and grammatical variants) means that the scope of a claim is to be interpreted to encompass the specified materials or steps recited in the claim” and those that do not materially alter the basic and novel characteristic(s)” of the claimed disclosure. Thus, the term “consisting essentially of” when used in a claim of this disclosure is not intended to be interpreted to be equivalent to “comprising.”

In the context of the disclosure, “corresponding to” or “corresponds to” means that when the amino acid sequences of a reference sequence are aligned with a second amino acid sequence (e.g. variant or homologous sequences), different from the reference sequence, the amino acids that “correspond to” certain enumerated positions in the second amino acid sequence are those that align with these positions in the reference amino acid sequence but that are not necessarily in the exact numerical positions relative to the particular reference amino acid sequence of the disclosure.

To “deliver” or “delivering” a composition or an insecticidal protein means that the composition or insecticidal protein comes in contact with an insect, which facilitates the oral ingestion of the composition or insecticidal protein, resulting in a toxic effect and control of the insect. The composition or insecticidal protein may be delivered in many recognized ways, e.g., through a transgenic plant expressing the insecticidal protein, formulated protein composition(s), sprayable protein composition(s), a bait matrix, or any other art-recognized toxin delivery system.

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability, or function of a protein. Identified by their high degree of conservation in aligned sequences of a family of protein homologues, they can be used as identifiers to determine if any polypeptide in question belongs to a previously identified polypeptide group.

An “engineered” protein of the disclosure refers to a protein that has a sequence that is different at least one amino acid position compared to at least one corresponding parent protein. An engineered protein can be a mutant protein that contains, e.g., one or more modifications such as deletions, additions, and/or substitutions of one or more amino acid positions relative to a parent protein. An engineered protein can be a chimeric protein and contain, e.g., one or more swapped or shuffled domains or fragments from at least two parent proteins.

“Effective insect-controlling amount” means that concentration of an insecticidal protein that inhibits, through a toxic effect, the ability of insects to survive, grow, feed and/or reproduce, or to limit insect-related damage or loss in crop plants. “Effective insect-controlling amount” may or may not mean killing the insects, although it preferably means killing the insects. A transgenic plant with “enhanced insecticidal properties” is a plant that is expresses a protein or proteins at effective insect-controlling amounts, so that, in some embodiments, the plant is insecticidal to an increased range of insect species, relative to a plant of the same kind which is not transformed. This increased range of insect species includes insect plant pests, such as coleopteran insect pests, e.g., Diabrotica virgifera virgifera (Western corn rootworm).

The term “event” refers to the original transformant and/or progeny of the transformant that include the heterologous DNA. The term “event” also refers to progeny produced by a sexual outcross between the transformant and another maize line. Even after repeated backcrossing to a recurrent parent, the inserted DNA and the flanking DNA from the transformed parent is present in the progeny of the cross at the same chromosomal location. The term “event” also refers to DNA from the original transformant comprising the inserted DNA and flanking genomic sequence immediately adjacent to the inserted DNA that would be expected to be transferred to a progeny as the result of a sexual cross of one parental line that includes the inserted DNA (e.g., the original transformant and progeny resulting from selfing) and a parental line that does not contain the inserted DNA. Typically, transformation of plant tissue produces multiple events, each of which represent insertion of a DNA construct into a different location in the genome of a plant cell.

“Expression cassette” as used herein means a nucleic acid sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The expression cassette comprising the nucleotide sequence of interest may have at least one of its components heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue, or organ, or stage of development.

An expression cassette comprising a nucleotide sequence of interest may be chimeric, meaning that at least one of its components is heterologous with respect to at least one of its other components. An expression cassette may also be one that comprises a native promoter driving its native gene; however, it has been obtained in a recombinant form useful for heterologous expression. Such usage of an expression cassette makes it so it is not naturally occurring in the cell into which it has been introduced.

An expression cassette also can optionally include a transcriptional and/or translational termination region (i.e., termination region) that is functional in plants. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked nucleotide sequence of interest, may be native to the plant host, or may be derived from another source (i.e., foreign, or heterologous to the promoter, the nucleotide sequence of interest, the plant host, or any combination thereof). Appropriate transcriptional terminators include, but are not limited to, the CAMV 35S terminator, the tml terminator, the nopaline synthase terminator and/or the pea rbcs E9 terminator. These can be used in both monocotyledons and dicotyledons. In addition, a coding sequence's native transcription terminator can be used. Any available terminator known to function in plants can be used in the context of this disclosure.

The term “expression” when used with reference to a polynucleotide, such as a gene, ORF or portion thereof, or a transgene in plants, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (e.g. if a gene encodes a protein), through “translation” of mRNA. Gene expression can be regulated at many stages in the process. For example, in the case of antisense or dsRNA constructs, respectively, expression may refer to the transcription of the antisense RNA only or the dsRNA only. In some embodiments, “expression” refers to the transcription and stable accumulation of sense (mRNA) or functional RNA. “Expression” may also refer to the production of protein.

A “gene” is a defined region that is located within a genome and comprises a coding nucleic acid sequence and typically also comprises other, primarily regulatory, nucleic acids responsible for the control of the expression, that is to say the transcription and translation, of the coding portion. A gene may also comprise other 5′ and 3′ untranslated sequences and termination sequences. Further elements that may be present are, for example, introns. The regulatory nucleic acid sequence of the gene may not normally be operatively linked to the associated nucleic acid sequence as found in nature and thus would be a chimeric gene.

The term “heterologous” when used in reference to a gene or a polynucleotide or a polypeptide refers to a gene or a polynucleotide or a polypeptide that is or contains a part thereof not in its natural environment (i.e., has been altered by the hand of man). For example, a heterologous gene may include a polynucleotide from one species introduced into another species. A heterologous gene may also include a polynucleotide native to an organism that has been altered in some way (e.g., located in a non-native genomic location, mutated, added in multiple copies, linked to a non-native promoter or enhancer polynucleotide, etc.). Heterologous genes further may comprise plant gene polynucleotides that comprise cDNA forms of a plant gene; the cDNAs may be expressed in either a sense (to produce mRNA) or anti-sense orientation (to produce an anti-sense RNA transcript that is complementary to the mRNA transcript). In one aspect of the disclosure, heterologous genes are distinguished from endogenous plant genes in that the heterologous gene polynucleotide are typically joined to polynucleotides comprising regulatory elements such as promoters that are not found naturally associated with the gene for the protein encoded by the heterologous gene or with plant gene polynucleotide in the chromosome, or are associated with portions of the chromosome not found in nature (e.g., genes expressed in loci where the gene is not normally expressed). Further, a “heterologous” polynucleotide refers to a polynucleotide not naturally associated with a host cell into which it is introduced, including non-naturally occurring multiple copies of a naturally occurring polynucleotide. A heterologous nucleic acid sequence or nucleic acid molecule may comprise a chimeric sequence such as a chimeric expression cassette, where the promoter and the coding region are derived from multiple source organisms. The promoter sequence may be a constitutive promoter sequence, a tissue-specific promoter sequence, a chemically-inducible promoter sequence, a wound-inducible promoter sequence, a stress-inducible promoter sequence, or a developmental stage-specific promoter sequence.

A “homologous” nucleic acid sequence is a nucleic acid sequence naturally associated with a host cell into which it is introduced.

The terms “increase”, “increasing”, “increased”, “enhance”, “enhanced”, “enhancing”, and “enhancement” and similar terms, as used herein, describe an elevation in control of a plant pest, e.g., by contacting a plant with a polypeptide of the disclosure (such as, for example, by transgenic expression or by topical application methods). The increase in control can be in reference to the level of control of the plant pest in the absence of the polypeptide of the disclosure (e.g., a plant that is not transgenically expressing the polypeptide or is not topically treated with the polypeptide). Thus in some embodiments, the terms “increase”, “increasing”, “increased”, “enhance”, “enhanced”, “enhancing”, and “enhancement” and similar terms can indicate an elevation of at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500% or more as compared to a suitable control (e.g., a plant, plant part, plant cell that is not contacted with the polypeptide of the disclosure).

The term “identity” or “identical” in the context of two nucleic acid or amino acid sequences, refers to the percentage of identical nucleotides or amino acids in a linear polynucleotide or amino acid sequence of a reference (“query”) sequence (or its complementary strand) as compared to a test (“subject”) sequence when the two sequences are globally aligned. Unless otherwise stated, sequence identity as used herein refers to the value obtained using the Needleman and Wunsch algorithm ((1970) J. Mol. Biol. 48:443-453) implemented in the EMBOSS Needle alignment tool using default matrix files EBLOSUM62 for protein with default parameters (Gap Open=10, Gap Extend=0.5, End Gap Penalty=False, End Gap Open=10, End Gap Extend=0.5) or DNAfull for nucleic acids with default parameters (Gap Open=10, Gap Extend=0.5, End Gap Penalty=False, End Gap Open=10, End Gap Extend=0.5); or any equivalent program thereof. EMBOSS Needle is available, e.g., from EMBL-EBI such as at the following website: ebi.ac.uk/Tools/psa/emboss_needle/ and as described in the following publication: “The EMBL-EBI search and sequence analysis tools APIs in 2019.” Madeira et al. Nucleic Acids Research, June 2019, 47(W1):W636-W641. The term “equivalent program” as used herein refers to any sequence comparison program that, for any two sequences in question, generates an alignment having identical nucleotide or amino acid residue matches and an identical percent sequence identity when compared to the corresponding alignment generated by EMBOSS Needle. In some embodiments, substantially identical nucleic acid or amino acid sequences may perform substantially the same function.

“Insecticidal” as used herein is defined as a toxic biological activity capable of controlling an insect pest, optionally but preferably by killing them.

In some embodiments, the polynucleotides or polypeptides of the disclosure are “isolated”. The term “isolated” polynucleotide or polypeptide is a polynucleotide or polypeptide that no longer exists in its natural environment. An isolated polynucleotide or polypeptide of the disclosure may exist in a purified form or may exist in a recombinant host such as in a transgenic bacteria or a transgenic plant. Therefore, for example, a claim to an “isolated” polynucleotide or polypeptide encompasses a nucleic acid molecule when the nucleic acid molecule is comprised within a transgenic plant genome.

The term “isolated”, when used in the context of the nucleic acid molecules or polynucleotides of the present disclosure, refers to a polynucleotide that is identified within and isolated/separated from its chromosomal polynucleotide context within the respective source organism. An isolated nucleic acid or polynucleotide is not a nucleic acid as it occurs in its natural context if it indeed has a naturally occurring counterpart. In contrast, non-isolated nucleic acids are nucleic acids such as DNA and RNA, which are found in the state they exist in nature. For example, a given polynucleotide (e.g., a gene) is found on the host cell chromosome in proximity to neighboring genes. The isolated nucleic acid molecule may be present in single-stranded or double-stranded form. Alternatively, it may contain both the sense and antisense strands (i.e., the nucleic acid molecule may be double-stranded). In some embodiments, the nucleic acid molecules of the present disclosure are isolated.

The term “motif” or “consensus sequence” or “signature” refers to a short, conserved region in the sequence of evolutionarily related proteins. Motifs are frequently highly conserved parts of domains, but may also include only part of the domain, or be located outside of conserved domain (if all of the amino acids of the motif fall outside of a defined domain).

A “native” or “wild type” nucleic acid, polynucleotide, nucleotide sequence, polypeptide or amino acid sequence refers to a naturally occurring or endogenous nucleic acid, polynucleotide, nucleotide sequence, polypeptide, or amino acid sequence.

A “nucleic acid molecule” or “nucleic acid” is a segment of single-stranded, double-stranded, or partially double-stranded DNA or RNA, or a hybrid thereof, that can be isolated or synthesized from any source. In the context of the present disclosure, the nucleic acid molecule is typically a segment of DNA. In some embodiments, the nucleic acid molecules of the disclosure are isolated nucleic acid molecules. In some embodiments, the nucleic acid molecules of the disclosure are comprised within a vector, a plant, a plant cell, or a bacterial cell. The terms also include reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A nucleic acid molecule can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically, or metabolically modified forms of polynucleotides, as well as the chemical forms of DNA and RNA characteristic of viruses and cells, including inter alia, simple, and complex cells.

The terms “nucleic acid,” “nucleic acid molecule,” and “polynucleotide” are used interchangeably herein.

“Operably linked” refers to the association of polynucleotides on a single nucleic acid molecule so that the function of one affects the function of the other. For example, a promoter is operably linked with a coding polynucleotide when it is capable of affecting the expression of that coding polynucleotide (i.e., that the coding polynucleotide is under the transcriptional control of the promoter). Coding polynucleotide in sense or antisense orientation can be operably linked to regulatory polynucleotides.

As used herein “pesticidal,” insecticidal,” and the like, refer to the ability of proteins of the disclosure to control a pest organism or an amount of one or more proteins of the disclosure that can control a pest organism.

The term “plant” includes reference to whole plants, plant organs, plant tissues (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen, and microspores. The class of plants, which can be used in the methods of the disclosure, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum. A particularly preferred plant is maize.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes, and embryos at various stages of development.

“Plant material” refers to leaves, stems, roots, flowers or flower parts, fruits, pollen, egg cells, zygotes, seeds, cuttings, cell or tissue cultures, or any other part or product of a plant.

A “plant organ” is a distinct and visibly structured and differentiated part of a plant such as a root, stem, leaf, flower bud, or embryo.

As used herein, “plant material,” “plant part” or “plant tissue” means plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, tubers, rhizomes, and the like. Any tissue of a plant in planta or in culture is included in the term “plant tissue.”.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

As used herein “plant sample” or “biological sample” refers to either intact or non-intact (e.g. milled seed or plant tissue, chopped plant tissue, lyophilized tissue) plant tissue. It may also be an extract comprising intact or non-intact seed or plant tissue. The biological sample or extract may be selected from the group consisting of corn flour, corn meal, corn syrup, corn oil, corn starch, and cereals manufactured in whole or in part to contain corn by-products.

A “polynucleotide of interest” or “nucleic acid of interest” refers to any polynucleotide which, when transferred to an organism, e.g., a plant, confers upon the organism a desired characteristic such as insect resistance, disease resistance, herbicide tolerance, antibiotic resistance, improved nutritional value, improved performance in an industrial process, production of a commercially valuable enzyme or metabolite, an altered reproductive capability, and the like.

A “portion” or a “fragment” of a polypeptide of the disclosure will be understood to mean an amino acid sequence or nucleic acid sequence of reduced length relative to a reference amino acid sequence or nucleic acid sequence of the disclosure. Such a portion or a fragment according to the disclosure may be, where appropriate, included in a larger polypeptide or nucleic acid of which it is a constituent (e.g., a tagged or fusion protein or an expression cassette). In some embodiments, the “portion” or “fragment” substantially retains the activity, such as insecticidal activity (e.g., at least 40%, 50%, 60%, 70%, 80%, 85%, 90%, 95% or even 100% of the activity) of the full-length protein or nucleic acid, or has even greater activity, e.g., insecticidal activity, than the full-length protein).

The terms “protein,” “peptide,” and “polypeptide” are used interchangeably herein.

The term “promoter,” as used herein, refers to a polynucleotide, usually upstream (5′) of the translation start site of a coding sequence, which controls the expression of the coding sequence by providing the recognition for RNA polymerase and other factors required for proper transcription. For example, a promoter may contain a region containing basal promoter elements recognized by RNA polymerase, a region containing the 5′ untranslated region (UTR) of a coding sequence, and optionally an intron.

As used herein, the term “recombinant” refers to a form of nucleic acid (e.g., DNA or RNA) or protein or an organism that would not normally be found in nature and as such was created by human intervention. As used herein, a “recombinant nucleic acid molecule” is a nucleic acid molecule comprising a combination of polynucleotides that would not naturally occur together and is the result of human intervention, e.g., a nucleic acid molecule that is comprised of a combination of at least two polynucleotides heterologous to each other, or a nucleic acid molecule that is artificially synthesized, for example, a polynucleotide synthesize using an assembled nucleotide sequence, and comprises a polynucleotide that deviates from the polynucleotide that would normally exist in nature, or a nucleic acid molecule that comprises a transgene artificially incorporated into a host cell's genomic DNA and the associated flanking DNA of the host cell's genome. Another example of a recombinant nucleic acid molecule is a DNA molecule resulting from the insertion of a transgene into a plant's genomic DNA, which may ultimately result in the expression of a recombinant RNA or protein molecule in that organism. As used herein, a “recombinant plant” is a plant that would not normally exist in nature, is the result of human intervention, and contains a transgene or heterologous nucleic acid molecule which may be incorporated into its genome. As a result of such genomic alteration, the recombinant plant is distinctly different from the related wild-type plant. A “recombinant” bacteria is a bacteria not found in nature that comprises a heterologous nucleic acid molecule. Such a bacteria may be created by transforming the bacteria with the nucleic acid molecule or by the conjugation-like transfer of a plasmid from one bacteria strain to another, whereby the plasmid comprises the nucleic acid molecule.

The terms “reduce,” “reduced,” “reducing,” “reduction,” “diminish,” and “suppress” (and grammatical variations thereof) and similar terms, as used herein, refer to a decrease in the survival, growth and/or reproduction of a plant pest, e.g., by contacting a plant with a polypeptide of the disclosure (such as, for example, by transgenic expression or by topical application methods). This decrease in survival, growth and/or reproduction can be in reference to the level observed in the absence of the polypeptide of the disclosure (e.g., a plant that is not transgenically expressing the polypeptide or is not topically treated with the polypeptide). Thus, in some embodiments, the terms “reduce,” “reduced,” “reducing,” “reduction,” “diminish,” and “suppress” (and grammatical variations thereof) and similar terms mean a decrease of at least about 5%, 10%, 15%, 20%, 25%, 30%, 35%, 40%, 45%, 50%, 55%, 60%, 65%, 70%, 75%, 80%, 85%, 90%, 95% or more as compared with a plant that is not contacted with a polypeptide of the disclosure (e.g., a plant that is not transgenically expressing the polypeptide or is not topically treated with the polypeptide). In representative embodiments, the reduction results in no or essentially no (i.e., an insignificant amount, e.g., less than about 10%, less than about 5% or even less than about 1%) detectable survival, growth and/or reproduction of the plant pest.

“Regulatory elements” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translational enhancer sequences, introns, terminators, and polyadenylation signal sequences. They include natural and synthetic sequences as well as sequences which may be a combination of synthetic and natural sequences. Regulatory sequences may determine expression level, the spatial and temporal pattern of expression and, for a subset of promoters, expression under inductive conditions (regulation by external factors such as light, temperature, chemicals, and hormones).

As used herein, “selectable marker” means a nucleotide sequence that when expressed imparts a distinct phenotype to the plant, plant part and/or plant cell expressing the marker and thus allows such transformed plants, plant parts and/or plant cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening (e.g., the R-locus trait).

“Stable transformation” or “stably transformed” as used herein means that a nucleic acid is introduced into a cell and integrates into the genome of the cell. As such, the integrated nucleic acid is capable of being inherited by the progeny thereof, more particularly, by the progeny of multiple successive generations. “Genome” as used herein also includes the nuclear and the plastid genome, and therefore includes integration of the nucleic acid into, for example, the chloroplast genome. Stable transformation as used herein can also refer to a transgene that is maintained extrachromosomally, for example, as a minichromosome.

“Synthetic” refers to a nucleotide sequence comprising bases or a structural feature(s) that is not present in the natural sequence. For example, an artificial sequence encoding a protein of the disclosure that resembles more closely the G+C content and the normal codon distribution of dicot or monocot plant genes is said to be synthetic.

A “target peptide” or “transit peptide” as used herein refers to a short (e.g., typically 3-70 amino acids long) polypeptide that when operably fused to a protein of interest directs the transport of the protein of interest to a specific region in the cell, including, for example, the nucleus, mitochondria, endoplasmic reticulum (ER), chloroplast, apoplast, peroxisome, or plasma membrane. The transit peptide can be operably fused to the N-terminus or the C-terminus of a protein, depending on the transit peptide of interest. In specific embodiments, the transit peptide is fused to the N-terminus of the polypeptide of interest. A transit peptide that directs a protein to the chloroplast is referred to as a chloroplast transit peptide or CTP. In specific embodiments, the chloroplast transit peptide is operably fused to the N-terminus of the polypeptide of interest.

As used herein, a protein of the disclosure that is “toxic” to an insect pest is meant that the protein functions as an orally active insect control agent to kill the insect pest, or the protein is able to disrupt or deter insect feeding, or causes growth inhibition to the insect pest, both of which may or may not cause death of the insect. When a toxic protein of the disclosure is delivered to an insect or an insect comes into oral contact with the toxic protein, the result is typically death of the insect, or the insect's growth is slowed, or the insect stops feeding upon the source that makes the toxic protein available to the insect.

The terms “toxin fragment” and “toxin portion” are used interchangeably herein to refer to a fragment or portion of a longer (e.g., full-length) insecticidal protein of the disclosure, where the “toxin fragment” or “toxin portion” retains insecticidal activity. For example, it is known in the art that native Cry proteins are expressed as protoxins that are processed at the N-terminal and C-terminal ends to produce a mature toxin. In some embodiments, the “toxin fragment” or “toxin portion” of a chimeric insecticidal protein of the disclosure is truncated at the N-terminus and/or C-terminus. In some embodiments, the “toxin fragment” or “toxin portion” is truncated at the N-terminus to remove part or all of the N-terminal peptidyl fragment, and optionally comprises at least about 400, 425, 450, 475, 500, 510, 520, 530, 540, 550, 560, 570, 580 or 590 contiguous amino acids of insecticidal protein specifically described herein or an amino acid sequence that is substantially identical thereto. Thus, in some embodiments, a “toxin fragment” or “toxin portion” of an insecticidal protein is truncated at the N-terminus (e.g., to omit part or all of the peptidyl fragment), for example, an N-terminal truncation of one amino acid or more than one amino acid, e.g., up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more amino acids. In some embodiments, a “toxin fragment” or “toxin portion” of an insecticidal protein is truncated at the C-terminus (e.g., to omit part or all of the protoxin tail), for example, a C-terminal truncation of one amino acid or more than one amino acid, e.g., up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 125, 150, 175, 200, 225, 250, 275, 300, 325, 350, 375, 400, 425, 450, 475, 500, 525, 550, 560 or more amino acids. In some embodiments, the “toxin fragment” or “toxin portion” comprises domains 1 and 2, and the core domain 3. In some embodiments, the “toxin fragment” or “toxin portion” is the mature (i.e., processed) toxin (e.g., Cry toxin).

“Transformation” is a process for introducing heterologous nucleic acid into a host cell or organism. In particular embodiments, “transformation” means the stable integration of a DNA molecule into the genome (nuclear or plastid) of an organism of interest. In some particular embodiments, the introduction into a plant, plant part and/or plant cell is via bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethylene glycol-mediated transformation, protoplast transformation, or any other electrical, chemical, physical and/or biological mechanism that results in the introduction of nucleic acid into the plant, plant part and/or cell thereof, or a combination thereof. Procedures for transforming plants are well known and routine in the art and are described throughout the literature. Non-limiting examples of methods for transformation of plants include transformation via bacterial-mediated nucleic acid delivery (e.g., via bacteria from the genus Agrobacterium), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (2002, Cell Mol Biol Lett 7:849-858 (2002)).

“Transformed” and “transgenic” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

The term “transgenic plant” includes reference to a plant into which a heterologous nucleic acid molecule has been introduced. Generally, the heterologous nucleic acid sequence is stably integrated within the genome such that the nucleic acid sequence is passed on to successive generations. The heterologous nucleic acid sequence may be integrated into the genome alone or as part of a recombinant expression cassette. “Transgenic” is used herein to include any cell, cell line, callus, tissue, plant part or plant, the genotype of which has been altered by the presence of a heterologous nucleic acid sequence, including those transgenics initially so altered as well as those created by sexual crosses or asexual propagation from the initial transgenic.

The term “vector” refers to a composition for transferring, delivering, or introducing a nucleic acid (or nucleic acids) into a cell. A vector comprises a nucleic acid molecule comprising the nucleotide sequence(s) to be transferred, delivered, or introduced. Example vectors include a plasmid, cosmid, phagemid, artificial chromosome, phage, or viral vector.

### Insecticidal Proteins, Polypeptides, Nucleic Acid Molecules

The present disclosure provides novel insecticidal proteins which have activity against Coleopterans, for example, Diabrotica virgifera virgifera (western corn rootworm; WCR), Diabrotica barberi (northern corn rootworm; NCR), and/or Diabrotica undecimpunctata howardi (southern corn rootworm; SCR) and/or other Diabrotica species including Diabrotica virgifera zeae (Mexican corn rootworm), and/or other Coleopteran insect pests such as Colorado Potato Beetle. The present disclosure also relates to nucleic acids whose expression results in insecticidal proteins of the disclosure, and to the making and using of the insecticidal proteins to control insect pests. In some embodiments, the expression of the nucleic acids results in insecticidal proteins that can be used to control Coleopteran insects such as western, northern and/or southern corn rootworm, particularly when expressed in a transgenic plant such as a transgenic corn plant.

In some embodiments, the present disclosure provides a polypeptide comprising an amino acid sequence that has at least 70% sequence identity (at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8, or at least 99.9% sequence identity) to any one of SEQ ID NOs:1-4, 26-29, or 30-473, wherein the polypeptide has insecticidal activity and/or insecticidal activity against a coleopteran pest (for example, corn root worm).

In some embodiments, the present disclosure provides a polypeptide comprising an amino acid sequence that has at least 90% sequence identity (at least 90%, at least 91%, at least 92%, at least 93%, at least 94, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8, or at least 99.9% sequence identity) to any one of SEQ ID NOs:1-4, 26-29, or 30-473, wherein the polypeptide has insecticidal activity and/or insecticidal activity against a coleopteran pest (for example, corn root worm).

In some embodiments, the polypeptide comprises any one of SEQ ID NOs:1-4, 26-29, 30-473.

In some embodiments, the polypeptide comprises one or more conserved protein domains based on Pfam data. The Pfam database is a database of protein families, which is described in Finn et al. (2014) Nucl. Acid Res. Database Issue 42:D222-D230 and is available online through InterPro at ebi.ac.uk/interpro/ (see, e.g., Paysan-Lafosse T, Blum M, Chuguransky S, Grego T, Pinto B L, Salazar G A, Bileschi M L, Bork P, Bridge A, Colwell L, Gough J, Haft D H, Letunid I, Marchler-Bauer A, Mi H, Natale D A, Orengo C A, Pandurangan A P, Rivoire C, Sigrist C J A, Sillitoe I, Thanki N, Thomas P D, Tosatto S C E, Wu C H, Bateman A. InterPro in 2022. Nucleic Acids Research, November 2022). Both the Pfam domain and the positions of the Pfam domain within a given SEQ ID NO are provided in Table 1 below. The start position is the amino acid position in the applicable SEQ ID NO where the domain starts; the end position is the amino acid position in the applicable SEQ ID NO where the domain ends. In specific embodiments, the polypeptide can comprise at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-4 and further comprises the applicable conserved domain(s) set forth in Table 1. For example, in one embodiment, the polypeptide will comprise at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-4, and further comprise 95%, 96%, 97%, 98%, 99% or 100% identity at the positions of the domain(s) specified in Table 1 below for any one of SEQ ID NOs: 1-4. Any of such polypeptides will retain insecticidal activity and/or insecticidal activity against a coleopteran pest (for example corn root worm).

TABLE 1
Pfam domains of Mv6_CRW_v1-v4

| | SEQ | Pfam domain | | Associated | | |
|---|---|---|---|---|---|---|
| Polypeptide | ID NO: | name | Pfam ID | InterPro ID | Start | End |
| Mv6_CRW_v1 | 1 | Endotoxin_N | PF03945 | IPR005639 | 21 | 117 |
| Mv6_CRW_v2 | 2 | Endotoxin_N | PF03945 | IPR005639 | 23 | 119 |
| Mv6_CRW_v3 | 3 | Endotoxin_N | PF03945 | IPR005639 | 21 | 117 |
| Mv6_CRW_v4 | 4 | Endotoxin_N | PF03945 | IPR005639 | 23 | 119 |

In some embodiments, the polypeptide is toxic to a coleopteran pest, e.g., a Diabrotica pest such as Western corn rootworm. In some embodiments, the polypeptide is derived from a particular fern, e.g., a Marsilea fern. In further embodiments, a polypeptide consisting of the amino acid sequence of any one of SEQ ID NOs: 1-4, 26-29, or 30-473 or differing from the amino acid sequence of any one of SEQ ID NOs: 1-4, 26-29, or 30-473 by substitution (e.g., conservative amino acid substitution), deletion, and/or addition of 1-10 amino acids (e.g., 1-10, 1-9, 1-8, 1-7, 1-6, 1-5, 1-4, 1-3, 1-2, or 1) is provided. A “conservative amino acid substitution” is one in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); polar, negatively charged residues and their amides (e.g., aspartic acid, asparagine, glutamic, acid, glutamine; uncharged polar side chains (e.g., glycine, asparagine, glutamine, serine, threonine, tyrosine, cysteine); small aliphatic, nonpolar or slightly polar residues (e.g., Alanine, serine, threonine, proline, glycine); nonpolar side chains (e.g., alanine, valine, leucine, isoleucine, proline, phenylalanine, methionine, tryptophan); large aliphatic, nonpolar residues (e.g., methionine, leucine, isoleucine, valine, cystine); beta-branched side chains (e.g., threonine, valine, isoleucine); aromatic side chains (e.g., tyrosine, phenylalanine, tryptophan, histidine); large aromatic side chains (e.g., tyrosine, phenylalanine, tryptophan). In some embodiments, said polypeptide is toxic to a coleopteran pest, e.g., a Diabrotica pest such as Western corn rootworm. In another embodiment, the disclosure provides a polypeptide consisting of the amino acid sequence of any one of any one of SEQ ID NOs: 1-4.

Dietary exposure is the major route by which humans can be exposed to insecticidal proteins expressed in transgenic plants. Acute oral mammalian toxicity and protein digestibility are the end points for EPA's human health risk assessment. Further scientific evidence of the safety of insecticidal proteins is that they have been shown to be rapidly degraded in vitro using simulated gastric fluids. For example, results of seven in vitro assays conducted with representative Cry1, Cry2, and Cry3 proteins establish that the proteins are rapidly degraded, typically within 30 seconds. These results support the broader conclusion that members of these groups of Cry proteins (that share significant amino acid sequence identity) are likely to be rapidly degraded following ingestion by humans. Similar tests are done for each transgenic protein expressed in plants. Another area of consideration is whether insecticidal proteins may induce an allergenic reaction. Demonstrated rapid in vitro degradation of the transgenic insecticidal protein should minimize the potential for such an occurrence. By comparison, food allergens generally persist in the in vitro gastrointestinal model, whereas common food proteins with no allergenic history degraded rapidly in simulated gastric fluid (Metcalfe et al. 1996).

A simulated gastric fluid (SGF) assay measures the in vitro digestibility of a test protein at tightly controlled conditions representative of the upper mammalian digestive tract. For example, bacterially produced test Cry protein (at a concentration of 0.5-5 mg/ml) was exposed to the enzyme pepsin (from porcine gastric mucosa, solubilized in 2 mg/ml NaCl, pH 1.2) at a ratio of 10 Units of pepsin activity/μg test protein over a time period of one hour at 37 °C. Samples were removed at 1, 2, 5, 10, 30, and 60 minute timepoints and immediately quenched with the addition of pre-heated (95 °C-2 minutes) stop buffer (65% 0.5M Sodium Bicarbonate pH 11, 35% Tricine Loading Buffer) to immediately render pepsin inactive, and returned to heat for an additional 5 minutes. Once the assay was complete, time point samples and controls (test protein alone, pepsin alone) were examined by SDS-PAGE on a 10-20% Tris-Tricine gel (with peptides visible down to 1 kDa) to track the kinetics and level of digestion performed by pepsin. If the test protein or a significant polypeptide fragment of the text protein is visible at, for example, the 5 and/or 10 minute timepoints, then it is not digestible or not completely digestible by the SGF assay, and may be scored qualitatively as “no”, or “not digestible”. If the test protein and any significant polypeptide fragment is not visible at, for example, the 5 minute timepoint, then it is digestible by the SGF assay, and may be scored qualitatively as “yes” or “digestible”.

The disclosed insecticidal proteins may therefore, in some embodiments, be modified to improve a property of the protein, e.g., its insecticidal activity, its solubility, its stability and/or its digestibility. For example, the disclosed insecticidal proteins may additionally comprise introduced protease cleavage sites and/or cysteine substitutions. The introduced protease cleavage site(s) and/or cysteine substitution(s) are not naturally occurring, and are introduced into the polypeptide sequence, as a substitution mutation or as an insertion or deletion mutation, or some combination thereof. The introduced protease cleavage site(s) may be introduced by the insertion of at least one leucine residue in a polypeptide sequence. In some embodiments, the polypeptides comprise cysteine substitution(s), e.g., substitution of cysteine with another amino acid such as alanine or leucine. The introduced mutation(s) may destabilize the polypeptide, so that a protease may gain access to a cleavage site which it previously did not have access to due to tight and/or stable folding of the protein, or to steric hindrance. The introduced protease cleavage site(s) may be an introduced mutation in the polypeptide sequence which is recognized by a protease, such as chymotrypsin, trypsin, or pepsin, as a site for proteolytic cleavage.

In some embodiments, the introduced protease cleavage site(s) may alter an existing protease cleavage site so that it is recognized by a different protease. Protease cleavage sites for chymotrypsin, trypsin, and pepsin are well-known in the art. Chymotrypsin preferentially cleaves peptide amide bonds where the carboxyl side of the amide bond (the P1 position) is a large hydrophobic amino acid (tyrosine, tryptophan, and phenylalanine). Trypsin cleaves peptide chains mainly at the carboxyl side of the amino acids lysine or arginine, except when either is followed by proline. Pepsin is most efficient in cleaving peptide bonds between hydrophobic and preferably aromatic amino acids such as phenylalanine, tryptophan, tyrosine, and leucine. These cleavage sites are the preferential cleavage sites and do not include all cleavage sites recognized by chymotrypsin, trypsin, or pepsin, and furthermore do not include all cleavage sites for all proteases.

Cysteines in proteins are frequently covalently bonded to other cysteine residues to form disulfide bonds. Disulfide bonds play an important role in the folding and stability of some proteins. A variant polypeptide may have an altered or less stable tertiary structure compared to wild-type polypeptide. For example, the introduced mutation may “loosen” the three dimensional folding of the polypeptide, thereby making a protease cleavage site that was previously inaccessible (and therefore not cleaved) accessible to a protease. This results in the introduced mutation introducing a protease cleavage site that did not exist in the unaltered polypeptide. In preferred embodiments, the mutation does not alter or does not significantly alter the activity, or the insecticidal activity, of the polypeptide against coleopteran pests.

Compositions are further provided that allow for a polypeptide of interest to be targeted to a cellular location of interest. Polynucleotides encoding a polypeptide of the disclosure may be fused to signal sequences which will direct the localization of the polypeptide to particular compartments of a prokaryotic or eukaryotic cell. For example, in E. coli, the expression of a protein of the disclosure can be directed to the periplasmic space. Examples of signal sequences or proteins (or fragments thereof) to which the polypeptides of the disclosure may be fused to direct the expression of the polypeptide to the periplasmic space of bacteria include, but are not limited to, the pelB signal sequence, the maltose binding protein (MBP) signal sequence, MBP, the ompA signal sequence, the signal sequence of the periplasmic E. coli heat-labile enterotoxin B-subunit and the signal sequence of alkaline phosphatase.

For expression of a protein of the disclosure in eukaryotic cells, target peptides or transit peptides are provided that direct the transport of a protein to a specific region in the cell, including the nucleus, mitochondria, endoplasmic reticulum (ER), chloroplast, apoplast, peroxisome and plasma membrane. For expression in plant cells, compositions are provided wherein the proteins of the disclosure can be expressed to reside in the cytosol or targeted to various organelles of the plant cell. For example, targeting a protein to the chloroplast may result in increased levels of expressed protein in a transgenic plant while preventing off-phenotypes from occurring. Targeting may also result in an increase in pest resistance efficacy in the transgenic event. Some target peptides are cleaved from the protein by signal peptidases after the proteins are transported. For targeting to the chloroplast, proteins contain transit peptides which are around 40-50 amino acids. For descriptions of the use of chloroplast transit peptides, see U.S. Pat. Nos. 5,188,642 and 5,728,925. Many chloroplast-localized proteins are expressed from nuclear genes as precursors and are targeted to the chloroplast by a chloroplast transit peptide (CTP). Examples of such isolated chloroplast proteins include, but are not limited to, those associated with the small subunit (SSU) of ribulose-1,5,-bisphosphate carboxylase, ferredoxin, ferredoxin oxidoreductase, the light-harvesting complex protein I and protein II, thioredoxin F, enolpyruvyl shikimate phosphate synthase (EPSPS), and transit peptides described in U.S. Pat. No. 7,193,133. It has been demonstrated in vivo and in vitro that non-chloroplast proteins may be targeted to the chloroplast by use of protein fusions with a heterologous CTP and that the CTP is sufficient to target a protein to the chloroplast. Incorporation of a suitable chloroplast transit peptide such as the Arabidopsis thaliana EPSPS CTP (CTP2) (see, Klee et al., Mol. Gen. Genet. 210:437-442, 1987) or the Petunia hybrida EPSPS CTP (CTP4) (see, della-Cioppa et al., Proc. Natl. Acad. Sci. USA 83:6873-6877, 1986) has been shown to target heterologous EPSPS protein sequences to chloroplasts in 5 transgenic plants (see, U.S. Pat. Nos. 5,627,061; 5,633,435; and 5,312,910; and EP 0218571; EP 189707; EP 508909; and EP 924299). Additional examples of CTPs include, but are not limited to Zea mays ssRUBISCO, Z. mays beta-glucosidase, Z. mays malate dehydrogenase, Z. mays thioredoxin M-type as described in US Patent Application Publication 2012/0304336. Additional CTPs of interest include those set forth in SEQ ID 18-25.

For targeting the chloroplast, a sequence encoding a chloroplast transit peptide is placed 5′ in operable linkage and in frame to a polynucleotide encoding the disclosed proteins that has been designed for optimal expression in plant cells. Such compositions can include a CTP operably linked to any one of SEQ ID NOs:1-4 or 30-473 or an active variant or fragment thereof. In one non-limiting embodiment, SEQ ID NO: 1 or a polypeptide having at least 90%, 91%, 92%, 93%, 94%, 95%, 96%, 97%, 98%, or 99% to SEQ ID NO:1 and retaining insecticidal activity and/or coleopteran insecticidal activity is operably linked to any one of SEQ ID NO: 19, 21, 23 or 25.

The disclosed insecticidal protein(s) have insecticidal activity against Coleopteran pests. In some embodiments, the insecticidal protein(s) has/have activity against one or more of the following Coleopteran pests: Diabrotica spp. such as D. barberi (northern corn rootworm), D. virgifera virgifera (western corn rootworm), D. undecimpunctata howardii (southern corn rootworm), D. balteata (banded cucumber beetle), D. undecimpunctata undecimpunctata (western spotted cucumber beetle), D. significata (3-spotted leaf beetle), D. speciosa (cucurbit beetle), D. virgifera zeae (Mexican corn rootworm), D. beniensis, D. cristata, D. curviplustalata, D. dissimilis, D. elegantula, D. emorsitans, D. graminea, D. hispanloe, D. lemniscata, D. linsleyi, D. milleri, D. nummularis, D. occlusal, D. porrecea, D. scutellata, D. tibialis, D. trifasciata and/or D. viridula, Leptinotarsa spp. such as L. decemlineata (Colorado potato beetle), Chrysomela spp. such as C. scripta (cottonwood leaf beetle), Hypothenemus spp. such as H. hampei (coffee berry borer), Sitophilus spp. such as S. zeamais (maize weevil), Epitrix spp. such as E. hirtipennis (tobacco flea beetle) and/or E. cucumeris (potato flea beetle), Phyllotreta spp. such as P. cruciferae (crucifer flea beetle) and/or P. pusilla (western black flea beetle), Anthonomus spp. such as A. eugenii (pepper weevil), Hemicrepidus spp. such as H. memnonius (wireworms), Melanotus spp. such as M. communis (wireworm), Ceutorhychus spp. such as C. assimilis (cabbage seedpod weevil), Phyllotreta spp. such as P. cruciferae (crucifer flea beetle), Aeolus spp. such as A. mellillus (wireworm), Aeolus spp. such as A. mancus (wheat wireworm), Horistonotus spp. such as H. uhlerii (sand wireworm), Sphenophorus spp. such as S. maidis (maize billbug), S. zeae (timothy billbug), S. parvulus (bluegrass billbug), and S. callosus (southern corn billbug), Phyllophaga spp. (White grubs), Chaetocnema spp. such as C. pulicaria (corn flea beetle), Popillia spp. such as P. japonica (Japanese beetle), Epilachna spp. such as E. varivestis (Mexican bean beetle), Cerotoma spp. such as C. trifurcate (Bean leaf beetle), Epicauta spp. such as E. pestifera and E. lemniscata (Blister beetles), Holotrichia spp. such as H. diomphalia Bates (Northeast larger black chafer), or any combination of the foregoing.

In some embodiments, the insecticidal protein(s) has/have insecticidal activity against D. virgifera virgifera (western corn rootworm) and D. barberi (northern corn rootworm).

In some embodiments, the insecticidal protein(s) have insecticidal activity against an insect pest or colony that has resistance to another insecticidal agent, including another insecticidal protein (such as, e.g., a Bt protein). In some embodiments, the insecticidal protein(s) has/have insecticidal activity against a pest (e.g., a western corn rootworm) that is resistant to a Cry3A protein (e.g., mCry3A, including without limitation maize event MIR604 or MZIR098 or eCry3.1Ab, including without limitation maize event 5307 or MZIR098), a Cry3B protein (e.g., Cry3Bb1, including without limitation maize event MON87411 or MON88017), a Cry34/35 protein (e.g., Cry34 and Cry35, including without limitation maize event 59122 or DP-4114), a IPD072 protein (e.g., IPD072Aa, including without limitation maize event DP-023211-2 or DP-051291-2), a IPD079 protein (e.g., IPD079Ea, including without limitation maize event DP0915635-4), a Cry75 protein (e.g., a Cry75Aa1, including without limitation maize event MON95275) or a Vip4D (e.g., a Vip4Da2, including without limitation maize event MON95275).

Each of the disclosed insecticidal protein(s) may also have insecticidal activity against Lepidopteran, Hemipteran, Dipteran, Lygus spp., and/or other piercing and sucking insects, for example of the order Orthoptera or Thysanoptera. In some embodiments, each of the disclosed insecticidal protein(s) have activity against one or more of the following Lepidopteran pests: Spodoptera spp. such as S. frugiperda (fall armyworm), S. littoralis (Egyptian cotton leafworm), S. ornithogalli (yellowstriped armyworm), S. praefica (western yellowstriped armyworm), S. eridania (southern armyworm), S. litura (Common cutworm/Oriental leafworm), S. cosmioides (black armyworm), S. exempta (African armyworm), S. mauritia (lawn armyworm) and/or S. exigua (beet armyworm), Ostrinia spp. such as O. nubilalis (European corn borer) and/or O. furnacalis (Asian corn borer), Plutella spp. such as P. xylostella (diamondback moth), Agrotis spp. such as A. ipsilon (black cutworm), A. segetum (common cutworm), A. gladiaria (claybacked cutworm), and/or A. orthogonia (pale western cutworm), Striacosta spp. such as S. albicosta (western bean cutworm), Helicoverpa spp. such as H. zea (corn earworm/soybean podworm), H. punctigera (native budworm), and/or H. armigera (cotton bollworm), Heliothis spp. such as H. virescens (tobacco budworm), Diatraea spp. such as D. grandiosella (southwestern corn borer) and/or D. saccharalis (sugarcane borer), Trichoplusia spp. such as T. ni (cabbage looper), Sesamia spp. such as S. nonagroides (Mediterranean corn borer), S. inferens (Pink stem borer) and/or S. calamistis (pink stem borer), Pectinophora spp. such as P. gossypiella (pink bollworm), Cochylis spp. such as C. hospes (banded sunflower moth), Manduca spp. such as M. sexta (tobacco hornworm) and/or M. quinquemaculata (tomato hornworm), Elasmopalpus spp. such as E. lignosellus (lesser cornstalk borer), Pseudoplusia spp. such as P. includens (soybean looper), Anticarsia spp. such as A. gemmatalis (velvetbean caterpillar), Plathypena spp. such as P. scabra (green cloverworm), Pieris spp. such as P. brassicae (cabbage butterfly), Papaipema spp. such as P. nebris (stalk borer), Pseudaletia spp. such as P. unipuncta (common armyworm), Peridroma spp. such as P. saucia (variegated cutworm), Keiferia spp. such as K. lycopersicella (tomato pinworm), Artogeia spp. such as A. rapae (imported cabbageworm), Phthorimaea spp. such as P. operculella (potato tuberworm), Chrysodeixis spp. such as C. includens (soybean looper), Feltia spp. such as F. ducens (dingy cutworm), Chilo spp. such as C. suppressalis (striped stem borer), C. agamemnon (oriental corn borer), C. venosatus (spotted borer), and C. partellus (spotted stalk borer), Cnaphalocrocis spp. such as C. medinalis (rice leaffolder), Conogethes spp. such as C. punctiferalis (Yellow peach moth), Mythimna spp. such as M. separata (Oriental armyworm), Athetis spp. such as A. lepigone (Two-spotted armyworm), Busseola spp. such as B. fusca (maize stalk borer), Etiella spp. such as E. zinckenella (pulse pod borer), Leguminivora spp. such as L. glycinivorella (soybean pod borer), Matsumuraeses spp. such as M. phaseoli (adzuki pod worm), Omiodes spp. such as O. indicata (Soybean leaffolder/Bean-leaf webworm), Rachiplusia spp. such as R. nu (sunflower Looper), Maruca spp. such as M. Testulalis Geyer (Bean pod borer), Monolepta spp. such as M. hieroglyphica (Double-spotted leaf beetle), or any combination of the foregoing. In some embodiments, the insecticidal protein(s) has/have activity against one or more of the following Hemiptera pests: Chinavia hilaris (green stink bug), Anasa tristis De Geer (squash bug), Blissus leucopterus (chinch bug), Corythuca gossypii Fabricius (cotton lace bug), Cyrtopeltis modesta Distant (tomato bug), Dysdercus suturellus Hernch-Schaffer (cotton stainer), Euschistus servus Say (brown stink bug), E. variolarius Palisot de Beauvois (one-spotted stink bug), Graptostethus spp. (complex of seed bugs), Leptoglossus corculus Say (leaf-footed pine seed bug), Lygus lineolaris Palisot de Beauvois (tarnished plant bug), L. Hesperus Knight (Western tarnished plant bug), L. pratensis Linnaeus (common meadow bug), L. rugulipennis Poppius (European tarnished plant bug), Lygocoris pabulinus Linnaeus (common green capsid), Nezara viridula Linnaeus (southern green stink bug), Oebalus pugnax Fabricius (rice stink bug), Oncopeltus fasciatus Dallas (large milkweed bug), Pseudatomoscelis seriatus Reuter (cotton fleahopper), Calocoris norvegicus Gmelin (strawberry bug), Orthops campestris Linnaeus, Plesiocoris rugicollis Fallen (apple capsid), Cyrtopeltis modestus Distant (tomato bug), Cyrtopeltis notatus Distant (suckfly), Spanagonicus albofasciatus Reuter (whitemarked fleahopper), Diaphnocoris chlorionis Say (honeylocust plant bug), Labopidicola allii Knight (onion plant bug), Pseudatomoscelis seriatus Reuter (cotton fleahopper), Adelphocoris rapidus Say (rapid plant bug), Poecilocapsus lineatus Fabricius (four-lined plant bug), Nysius ericae Schilling (false chinch bug), Nysius raphanus Howard (false chinch bug), Nezara viridula Linnaeus (Southern green stink bug), Eurygaster spp., Coreidae spp., Pyrrhocoridae spp., Tinidae spp., Blostomatidae spp., Reduviidae spp. and Cimicidae spp., or any combination of the foregoing. In some embodiments, the insecticidal protein(s) has/have activity against one or more of the following Diptera pests: Liriomyza spp. such as L. trifolii (leafminer) and L. sativae (vegetable leafminer), Scrobipalpula spp. such as S. absoluta (tomato leafminer), Delia spp. such as D. platura (seedcorn maggot), D. brassicae (cabbage maggot) and D. radicum (cabbage root fly), Psilia spp. such as P. rosae (carrot rust fly), Tetanops spp. such as T. myopaeformis (sugarbeet root maggot), or any combination of the foregoing. In some embodiments, the insecticidal protein(s) has/have activity against one or more of the following Orthoptera pests: Melanoplus spp. such as M. differentialis (Differential grasshopper), M. femurrubrum (Redlegged grasshopper), M. bivittatus (Twostriped grasshopper), or any combination of the foregoing. In some embodiments, the insecticidal protein(s) has/have activity against one or more of the following Thysanoptera pests: Frankliniella spp. such as F. occidentalis (western flower thrips) and F. fusca (tobacco thrips), and Thrips spp. such as T. tabaci (onion thrips), T. palmi (melon thrips), or any combination of the foregoing.

The disclosed insecticidal protein(s) may also have insecticidal activity against any one or more of the following: Phyllophaga spp., Rhopalosiphum maidis, Pratylenchus penetrans, Melanotus cribulosus, Cyclocephala lurida, Limonius californicus, Tetranychus urticae, Haplothrips aculeatus, Tetranychus truncates, Anomala corpulenta, Oedaleus infernalis, Frankliniella tenuicornis, Tetranychus cinnabarinus, Aiolopus thalassinus tamulus, Trachea tokionis, Laodelphax striatellus, Holotrichia oblita, Dichelops furcatus, Diloboderus abderu, Dalbulus maidis, Astylus variegathus, Scaptocoris castanea, Locusta migratoria manilensis, Agriotes lineatus, Peregrinus maidis, Oscinella frit, Frankliniella williamsi, Zyginidia manaliensis, Atherigona soccata, Nicentrites testaceipes, Myllocerus undecimpustulatus, Atherigona naquii, Amsecta albistriga, Plodia interpuctella, Melanotus caudex, Microtermes spp., Atherigona oryzae, Tanymecus dilaticollis, Delphacodes kuschelli, Lepidiota stigma, Phyllophaga hellery, Tribolium castaneum, Pelopidas mathias, Oxya chinensis (Thunberg), Stenocranus pacificus, Scutigerella immaculata, Chrysodeixis chalcites, Euproctis sp. (Lymantriidae), Phyllotreata spp. (undulata), Reptalus panzer, Cyrtacanthacris tartarica Linnaeus, Orgyia postica, Dactylispa lameyi, Patanga succincta Johanson, Tetranychus spp., Calomycterus sp., Adoretus compressus Weber, and Paratetranychus stickney.

The disclosed insecticidal proteins may also be active against nematodes. The term “nematode” as used herein encompasses any organism that is now known or later identified that is classified in the animal kingdom, phylum Nematoda, including without limitation nematodes within class Adenophorea (including for example, orders Enoplida, Isolaimida, Mononchida, Dorylaimida, Trichocephalida, Mermithida, Muspiceida, Araeolaimida, Chromadorida, Desmoscolecida, Desmodorida and Monhysterida) and/or class Secernentea (including, for example, orders Rhabdita, Strongylida, Ascaridida, Spirurida, Camallanida, Diplogasterida, Tylenchida and Aphelenchida).

Nematodes include but are not limited to parasitic nematodes such as root-knot nematodes, cyst nematodes and/or lesion nematodes. Exemplary genera of nematodes according to the present disclosure include but are not limited to, Meloidogyne (root-knot nematodes), Heterodera (cyst nematodes), Globodera (cyst nematodes), Radopholus (burrowing nematodes), Rotylenchulus (reniform nematodes), Pratylenchus (lesion nematodes), Aphelenchoides (foliar nematodes), Helicotylenchus (spiral nematodes), Hoplolaimus (lance nematodes), Paratrichodorus (stubby-root nematodes), Longidorus, Nacobbus (false root-knot nematodes), Subanguina, Belonlaimus (sting nematodes), Criconemella, Criconemoides (ring nematodes), Ditylenchus, Dolichodorus, Hemicriconemoides, Hemicycliophora, Hirschmaniella, Hypsoperine, Macroposthonia, Melinius, Punctodera, Quinisulcius, Scutellonema, Xiphinema (dagger nematodes), Tylenchorhynchus (stunt nematodes), Tylenchulus, Bursaphelenchus (round worms), and any combination thereof.

Exemplary plant parasitic nematodes according to the present disclosure include, but are not limited to, Belonolaimus gracilis, Belonolaimus longicaudatus, Bursaphelenchus xylophilus (pine wood nematode), Criconemoides ornata, Ditylenchus destructor (potato rot nematode), Ditylenchus dipsaci (stem and bulb nematode), Globodera pallida (potato cyst nematode), Globodera rostochiensis (golden nematode), Heterodera glycines (soybean cyst nematode), Heterodera schachtii (sugar beet cyst nematode); Heterodera zeae (corn cyst nematode), Heterodera avenae (cereal cyst nematode), Heterodera carotae, Heterodera trifolii, Hoplolaimus columbus, Hoplolaimus galeatus, Hoplolaimus magnistylus, Longidorus breviannulatus, Meloidogyne arenaria, Meloidogyne chitwoodi, Meloidogyne hapla, Meloidogyne incognita, Meloidogyne javanica, Mesocriconema xenoplax, Nacobbus aberrans, Naccobus dorsalis, Paratrichodorus christiei, Paratrichodorus minor, Pratylenchus brachyurus, Pratylenchus crenatus, Pratylenchus hexincisus, Pratylenchus neglectus, Pratylenchus penetrans, Pratylenchus projectus, Pratylenchusscribneri, Pratylenchus tenuicaudatus, Pratylenchus thornei, Pratylenchus zeae, Punctodera chaccoensis, Quinisulcius acutus, Radopholus similis, Rotylenchulus reniformis, Tylenchorhynchus dubius, Tylenchulus semipenetrans (citrus nematode), Siphinema americanum, X. Mediterraneum, and any combination of the foregoing.

The disclosure also encompasses antibodies that specifically bind to the insecticidal proteins of the disclosure. The antibody can optionally be a monoclonal antibody or a polyclonal antisera. Such antibodies may be produced using standard immunological techniques for production of polyclonal antisera and, if desired, immortalizing the antibody-producing cells of the immunized host for sources of monoclonal antibody production. Techniques for producing antibodies to any substance of interest are well known, e.g., as described in Harlow and Lane (1988. Antibodies a laboratory manual. pp. 726. Cold Spring Harbor Laboratory) and as in Goding (Monoclonal Antibodies: Principles & practice. 1986. Academic Press, Inc., Orlando, FL). The present disclosure also encompasses an insecticidal protein that cross-reacts with an antibody, particularly a monoclonal antibody, raised against one or more of the insecticidal proteins of the present disclosure.

The antibodies according to the disclosure are useful, e.g., in immunoassays for determining the amount or presence of an insecticidal protein of the disclosure or an antigenically related polypeptide, e.g., in a biological sample. Such assays are also useful in quality-controlled production of compositions containing one or more of the insecticidal proteins of the disclosure or an antigenically related polypeptide. In addition, the antibodies can be used to assess the efficacy of recombinant production of one or more of the proteins of the disclosure or an antigenically related polypeptide, as well as for screening expression libraries for the presence of a nucleotide sequence encoding one or more of the proteins of the disclosure or an antigenically related polypeptide. Antibodies further find use as affinity ligands for purifying or isolating any one or more of the proteins of the disclosure or an antigenically related polypeptide.

In some embodiments, nucleic acid molecules which encode the polypeptides of the disclosure are provided. In some embodiments, the nucleic acid molecules are codon-optimized, e.g., as further described herein. In some embodiments the coding sequence is at least 80% identical to (at least 81%, at least 82%, at least 83%, at least 84%, at least 85%, at least 86%, at least 87%, at least 88%, at least 89%, at least 90%, at least 91%, at least 92%, at least 93%, at least 94, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8, or at least 99.9% identical) to any one of SEQ ID NOs: 5-9. In other embodiments, the coding sequence comprises or consists of the sequence of any one of SEQ ID NOs: 5-9. In some embodiments, the coding sequence comprises a degenerated nucleotide sequence encoding the same amino acid sequence of any one of SEQ ID NOs: 5-9.

### Expression Cassettes and Vectors

In some aspects, the disclosure provides expression cassettes and vectors that encode the insecticidal proteins of the disclosure. In some embodiments, coding sequences comprising synthetic nucleotide sequences that are codon optimized for expression in a plant (for example, a transgenic monocot plant host or a transgenic dicot plant host, such as a corn or soy plant). In some embodiments, the nucleotide coding sequence is partially or completely synthetic. In representative embodiments, for expression in transgenic plants, such as corn or soy, the nucleotide sequences of the disclosure are modified and/or optimized. For example, although in many cases genes from microbial organisms can be expressed in plants at high levels without modification, low expression in transgenic plants may result from microbial nucleotide sequences having codons that are not preferred in plants. It is known in the art that living organisms have specific preferences for codon usage, and the codons of the nucleotide sequences described in this disclosure can be changed to conform with plant preferences, while maintaining the amino acids encoded thereby. A table of codon usage for maize is provided below. In some embodiments, at least 50%, at least 60%, at least 70%, at least 80%, at least 90%, at least 95% or at least 99% of the codons in the nucleotide coding sequence correspond to those with the highest frequency according to the below table. It is understood that it might not be possible to use all of the highest frequency codons to encode a desired amino acid sequence as it could introduce, e.g., unintended splice sites or out-of-frame open reading frames.

TABLE 2
Preferred codon usage in Maize

| Codon | Amino acid | Frequency in maize |
|---|---|---|
| GCA | ALA | 5.44% |
| GCC | ALA | 48.50% |
| GCG | ALA | 38.39% |
| GCT | ALA | 7.67% |
| AGA | ARG | 2.56% |
| AGG | ARG | 19.76% |
| CGA | ARG | 2.44% |
| CGC | ARG | 47.88% |
| CGG | ARG | 22.81% |
| CGT | ARG | 4.55% |
| AAC | ASN | 91.94% |
| AAT | ASN | 8.06% |
| GAC | ASP | 89.18% |
| GAT | ASP | 10.82% |
| TGC | CYS | 93.44% |
| TGT | CYS | 6.56% |
| CAA | GLN | 7.16% |
| CAG | GLN | 92.84% |
| GAA | GLU | 8.91% |
| GAG | GLU | 91.09% |
| GGA | GLY | 6.65% |
| GGC | GLY | 61.19% |
| GGG | GLY | 24.80% |
| GGT | GLY | 7.36% |
| CAC | HIS | 89.53% |
| CAT | HIS | 10.47% |
| ATA | ILE | 4.81% |
| ATC | ILE | 86.31% |
| ATT | ILE | 8.88% |
| CTA | LEU | 2.26% |
| CTC | LEU | 48.67% |
| CTG | LEU | 39.74% |
| CTT | LEU | 5.15% |
| TTA | LEU | 0.52% |
| TTG | LEU | 3.67% |
| AAA | LYS | 6.20% |
| AAG | LYS | 93.80% |
| ATG | MET | 100.00% |
| TTC | PHE | 93.82% |
| TTT | PHE | 6.18% |
| CCA | PRO | 7.35% |
| CCC | PRO | 37.30% |
| CCG | PRO | 46.13% |
| CCT | PRO | 9.23% |
| AGC | SER | 27.02% |
| AGT | SER | 1.99% |
| TCA | SER | 3.09% |
| TCC | SER | 39.54% |
| TCG | SER | 23.25% |
| TCT | SER | 5.11% |
| ACA | THR | 5.47% |
| ACC | THR | 51.49% |
| ACG | THR | 37.03% |
| ACT | THR | 6.01% |
| TGG | TRP | 100.00% |
| TAC | TYR | 93.66% |
| TAT | TYR | 6.34% |
| GTA | VAL | 2.19% |
| GTC | VAL | 43.68% |
| GTG | VAL | 49.05% |
| GTT | VAL | 5.09% |
| TAA | STOP | 14.49% |
| TAG | STOP | 29.69% |
| TGA | STOP | 55.82% |

Furthermore, it is known in the art that high expression in plants, for example corn plants, can be achieved from coding sequences that have at least about 35% GC content, or at least about 45% GC content, or at least about 50% GC content, or at least about 60% GC content. Accordingly, in some embodiments, a nucleic acid sequence of the disclosure is codon-optimized for expression in plants by reducing the AT content of the nucleic acid relative to the wild-type sequence (e.g., SEQ ID NOs: 5-8), such as to achieve at least 35%, at least 45%, at least 50% or at least 60% GC content. Microbial nucleotide sequences that have low GC contents may express poorly in plants. Although certain nucleotide sequences can be adequately expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17:477-498 (1989)). In addition, in some embodiments, the nucleotide sequence is modified to remove illegitimate splice sites that may cause message truncation or to remove in-frame or out-of-frame start codons that would result in unintended open reading frames. Such modifications to the nucleotide sequences can be made using well known techniques of site directed mutagenesis, PCR, and synthetic gene construction using the methods described, for example, in U.S. Pat. Nos. 5,625,136; 5,500,365 and 6,013,523.

In some embodiments, the disclosure provides synthetic coding sequences or polynucleotides made according to the procedure disclosed in U.S. Pat. No. 5,625,136. In this procedure, maize preferred codons, i.e., the single codon that most frequently encodes that amino acid in maize, are used. It is recognized that codons optimized for expression in one plant species will also function in other plant species but possibly not at the same level as the plant species for which the codons were optimized. In this manner, the nucleotide sequences can be optimized for expression in any plant. It is recognized that all or any part of a nucleotide sequence may be optimized or synthetic. That is, a polynucleotide may comprise a nucleotide sequence that is part native sequence and part codon optimized sequence.

In representative embodiments, a polynucleotide of the disclosure is an isolated polynucleotide. In some embodiments, a polynucleotide of the disclosure is a recombinant polynucleotide.

In some embodiments, a heterologous promoter is operably linked to a nucleic acid comprising, consisting essentially of or consisting of a coding sequence that encodes a protein of the disclosure that is toxic to a coleopteran pest. Promoters can include, for example, constitutive, inducible, temporally regulated, developmentally regulated, chemically regulated, tissue-preferred and/or tissue-specific promoters. In particular aspects, a promoter useful with the disclosure is a promoter capable of initiating transcription of a nucleotide sequence in a plant cell, e.g., in a cell of a monocot (e.g., maize or rice) or dicot (e.g., soybean, cotton) plant.

In some embodiments, the heterologous promoter is a plant-expressible promoter (e.g., monocot expressible or dicto expressible). For example, without limitation, the plant-expressible promoter can be selected from the group of promoters consisting of ubiquitin, cestrum yellow virus, corn TrpA, OsMADS 6, maize H3 histone, bacteriophage T3 gene 9 5′ UTR, corn sucrose synthetase 1, corn alcohol dehydrogenase 1, corn light harvesting complex, corn heat shock protein, maize mtl, pea small subunit RuBP carboxylase, rice actin, rice cyclophilin, Ti plasmid mannopine synthase, Ti plasmid nopaline synthase, petunia chalcone isomerase, bean glycine rich protein 1, potato patatin, lectin, CaMV 35S and S-E9 small subunit RuBP carboxylase promoter.

Although many promoters from dicotyledons have been shown to be operational in monocotyledons and vice versa, in some embodiments, dicotyledonous promoters are selected for expression in dicotyledons, and monocotyledonous promoters for expression in monocotyledons. However, there is no restriction to the provenance of selected promoters; it is sufficient that they are operational in driving the expression of the nucleotide sequences in the desired cell.

The choice of promoter can vary depending on the temporal and spatial requirements for expression, and also depending on the host cell to be transformed. Thus, for example, expression of the nucleotide sequences of the disclosure can be in any plant and/or plant part, (e.g., in leaves, in stalks or stems, in ears, in inflorescences (e.g., spikes, panicles, cobs, etc.), in roots, seeds and/or seedlings, and the like). For example, where expression in a specific tissue or organ is desired, a tissue-specific or tissue-preferred promoter can be used (e.g., a root specific/preferred promoter). For example, where expression is not desired in a specific tissue or organ, a tissue-free promoter can be used. In some embodiments, a “pollen-free” promoter is provided which results in low or no detectable gene expression in the pollen of the target plant species. In contrast, where expression in response to a stimulus is desired a promoter inducible by stimuli or chemicals can be used. Where continuous expression at a relatively constant level is desired throughout the cells of a plant a constitutive promoter can be chosen.

Promoters useful with the disclosure include, but are not limited to, those that drive expression of a nucleotide sequence constitutively, those that drive expression when induced, and those that drive expression in a tissue- or developmentally-specific manner. These various types of promoters are known in the art.

Suitable constitutive promoters include, for example, CaMV 35S promoter (Odell et al., Nature 313:810-812, 1985); Arabidopsis At6669 promoter (see PCT Publication No. WO04081173A2); maize Ubi 1 (Christensen et al., Plant Mol. Biol. 18:675-689, 1992); rice actin (McElroy et al., Plant Cell 2:163-171, 1990); pEMU (Last et al., Theor. Appl. Genet. 81:581-588, 1991); CaMV 19S (Nilsson et al., Physiol. Plant 100:456-462, 1997); GOS2 (de Pater et al., Plant J November; 2(6):837-44, 1992); ubiquitin (Christensen et al., Plant Mol. Biol. 18: 675-689, 1992); Rice cyclophilin (Bucholz et al., Plant Mol Biol. 25(5):837-43, 1994); Maize H3 histone (Lepetit et al., Mol. Gen. Genet. 231: 276-285, 1992); Actin 2 (An et al., Plant J. 10(i); 107-121, 1996), constitutive root tip CT2 promoter (see PCT application No. IL/2005/000627) and Synthetic Super MAS (Ni et al., The Plant Journal 7: 661-76, 1995). Other constitutive promoters include those in U.S. Pat. Nos. 5,659,026, 5,608,149; 5,608,144; 5,604,121; 5,569,597: 5,466,785; 5,399,680; 5,268,463; and 5,608,142.

Tissue-specific or tissue-preferential promoters useful for the expression of the polypeptides of the disclosure in plants, optionally maize, include those that direct expression in root, pith, leaf or pollen. Suitable tissue-specific promoters include, but not limited to, leaf-specific promoters (such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993), seed-preferred promoters (e.g., from seed specific genes; Simon, et al., Plant Mol. Biol. 5. 191, 1985; Scofield, et al., J. Biol. Chem. 262: 12202, 1987; Baszczynski, et al., Plant Mol. Biol. 14: 633, 1990), Brazil Nut albumin (Pearson et al., Plant Mol. Biol. 18: 235-245, 1992), legumin (Ellis, et al. Plant Mol. Biol. 10: 203-214, 1988), Glutelin (Takaiwa, et al., Mol. Gen. Genet. 208: 15-22, 1986; Takaiwa, et al., FEBS Letts. 221: 43-47, 1987), Zein (Matzke et al., Plant Mol Biol, 143).323-32 1990), napA (Stalberg, et al., Planta 199: 515-519, 1996), Wheat SPA (Albanietal, Plant Cell, 9: 171-184, 1997), sunflower oleosin (Cummins, et al., Plant Mol. Biol. 19: 873-876, 1992)], endosperm specific promoters (e.g., wheat LMW and HMW, glutenin-1 (Mol Gen Genet 216:81-90, 1989; NAR 17:461-2), wheat a, b and g gliadins (EMB03:1409-15, 1984), Barley ltrl promoter, barley B1, C, D hordein (Theor Appl Gen 98:1253-62, 1999; Plant J 4:343-55, 1993; Mol Gen Genet 250:750-60, 1996), Barley DOF (Mena et al., The Plant Journal, 116(1): 53-62, 1998), Biz2 (EP99106056.7), Synthetic promoter (Vicente-Carbajosa et al., Plant J. 13: 629-640, 1998), rice prolamin NRP33, rice-globulin Glb-1 (Wu et al., Plant Cell Physiology 39(8) 885-889, 1998), rice alpha-globulin REB/OHP-1 (Nakase et al. Plant Mol. Biol. 33: 513-522, 1997), rice ADP-glucose PP (Trans Res 6:157-68, 1997), maize ESR gene family (Plant J 12:235-46, 1997), sorgum gamma-kafirin (Plant Mol. Biol 32:1029-35, 1996)], embryo specific promoters (e.g., rice OSH1; Sato et al., Proc. Nati. Acad. Sci. USA, 93: 8117-8122), KNOX (Postma-Haarsma of al, Plant Mol. Biol. 39:257-71, 1999), rice oleosin (Wu et at, J. Biochem., 123:386, 1998)] flower-specific promoters, for example, AtPRP4, chalene synthase (chsA) (Van der Meer, et al., Plant Mol. Biol. 15, 95-109, 1990), LAT52 (Twell et al., Mol. Gen Genet. 217:240-245; 1989), apetala-3, and promoters specific for plant reproductive tissues (e.g., OsMADS promoters; U.S. Patent Publication 2007/0006344).

Examples of promoters suitable for preferential expression in green tissue include many that regulate genes involved in photosynthesis and many of these have been cloned from both monocotyledons and dicotyledons. One such promoter is the maize PEPC promoter from the phosphoenol carboxylase gene (Hudspeth & Grula, Plant Molec. Biol. 12:579-589 (1989)). Another promoter for root specific expression is that described by de Framond (FEBS 290:103-106 (1991) or U.S. Pat. No. 5,466,785). Another promoter useful in the disclosure is the stem specific promoter described in U.S. Pat. No. 5,625,136, which naturally drives expression of a maize trpA gene.

In addition, promoters functional in plastids can be used. Non-limiting examples of such promoters include the bacteriophage T3 gene 9 5′ UTR and other promoters disclosed in U.S. Pat. No. 7,579,516. Other promoters useful with the disclosure include but are not limited to the S-E9 small subunit RuBP carboxylase promoter and the Kunitz trypsin inhibitor gene promoter (Kti3).

In some embodiments, inducible promoters can be used. Thus, for example, chemical-regulated promoters can be used to modulate the expression of a gene in a plant through the application of an exogenous chemical regulator. Regulation of the expression of nucleotide sequences of the disclosure via promoters that are chemically regulated enables the polypeptides of the disclosure to be synthesized only when the crop plants are treated with the inducing chemicals. Depending upon the objective, the promoter may be a chemical-inducible promoter, where application of a chemical induces gene expression, or a chemical-repressible promoter, where application of the chemical represses gene expression. Examples of such technology for chemical induction of gene expression is detailed in published application EP 0 332 104 and U.S. Pat. No. 5,614,395.

Chemical inducible promoters are known in the art and include, but are not limited to, the maize In2-2 promoter, which is activated by benzene sulfonamide herbicide safeners, the maize GST promoter, which is activated by hydrophobic electrophilic compounds that are used as pre-emergent herbicides, the tobacco PR-1 a promoter, which is activated by salicylic acid (e.g., the PR1a system), steroid steroid-responsive promoters (see, e.g., the glucocorticoid-inducible promoter in Schena et al. (1991) Proc. Natl. Acad. Sci. USA 88, 10421-10425 and McNellis et al. (1998) Plant J. 14, 247-257), tetracycline-inducible and tetracycline-repressible promoters (see, e.g., Gatz et al. (1991) Mol. Gen. Genet. 227, 229-237, and U.S. Pat. Nos. 5,814,618 and 5,789,156), Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters.

Other non-limiting examples of inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston et al. (1988) Genetics 119:185-197), the MPI proteinase inhibitor promoter (Cordero et al. (1994) Plant J. 6:141-150), and the glyceraldehyde-3-phosphate dehydrogenase promoter (Kohler et al. (1995) Plant Mol. Biol. 29:1293-1298; Martinez et al. (1989) J. Mol. Biol. 208:551-565; and Quigley et al. (1989) J. Mol. Evol. 29:412-421). Also included are the benzene sulphonamide-inducible (U.S. Pat. No. 5,364,780) and alcohol-inducible (Int'l Patent Application Publication Nos. WO 97/06269 and WO 97/06268) systems and glutathione S-transferase promoters. Likewise, one can use any of the inducible promoters described in Gatz (1996) Current Opinion Biotechnol. 7:168-172 and Gatz (1997) Annu. Rev. Plant Physiol. Plant Mol. Biol. 48:89-108. Other chemically inducible promoters useful for directing the expression of the nucleotide sequences of this disclosure in plants are disclosed in U.S. Pat. No. 5,614,395. Chemical induction of gene expression is also detailed in EP 0 332 104 (to Ciba-Geigy) and U.S. Pat. No. 5,614,395.

Another category of promoters useful in the disclosure are wound inducible promoters. Examples of promoters of this kind include those described by Stanford et al. Mol. Gen. Genet. 215:200-208 (1989), Xu et al. Plant Molec. Biol. 22:573-588 (1993), Logemann et al. Plant Cell 1:151-158 (1989), Rohrmeier & Lehle, Plant Molec. Biol. 22:783-792 (1993), Firek et al. Plant Molec. Biol. 22:129-142 (1993), and Warner et al. Plant J. 3:191-201 (1993).

In some embodiments, a recombinant vector is provided which comprises a nucleic acid molecule or an expression cassette of the disclosure. Certain vectors for use in transformation of plants and other organisms are known in the art. In other embodiments, non-limiting examples of a vector include a plasmid, cosmid, phagemid, artificial chromosome, phage or viral vector. In some embodiments, the vector is plant vector, e.g., for use in transformation of plants. In some embodiments, the vector is a bacterial vector, e.g., for use in transformation of bacteria. Suitable vectors for plants, bacteria and other organisms are known in the art.

Some embodiments are directed to expression cassettes designed to express the polynucleotides and nucleic acid molecules of the disclosure. In some embodiments, an expression cassette comprises a nucleic acid molecule having at least a control sequence operatively linked to a nucleotide sequence of interest, e.g. a nucleotide sequence encoding an insecticidal protein of the disclosure. In this manner, for example, plant promoters operably linked to the nucleotide sequences to be expressed are provided in expression cassettes for expression in a plant, plant part or plant cell.

An expression cassette comprising a polynucleotide of interest may be chimeric, meaning that at least one of its components is heterologous with respect to at least one other of its other components. An expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation event.

In addition to the promoters operatively linked to the nucleotide sequences of the disclosure, an expression cassette of this disclosure also can include other regulatory sequences. Regulatory sequences include, but are not limited to, enhancers, introns, translation leader sequences, termination signals, and polyadenylation signal sequences.

In some embodiments, an expression cassette can also include polynucleotides that encode other desired traits in addition to the disclosed proteins. Such expression cassettes comprising the stacked traits may be used to create plants, plant parts or plant cells having a desired phenotype with the stacked traits (i.e., molecular stacking). Such stacked combinations in plants can also be created by other methods including, but not limited to, cross breeding plants by any conventional methodology. If stacked by genetically transforming the plants, the nucleotide sequences of interest can be combined at any time and in any order. For example, a transgenic plant comprising one or more desired traits can be used as the target to introduce further traits by subsequent transformation. The additional nucleotide sequences can be introduced simultaneously in a co-transformation protocol with a nucleotide sequence, nucleic acid molecule, nucleic acid construct, or composition of this disclosure, provided by any combination of expression cassettes. For example, if two nucleotide sequences will be introduced, they can be incorporated in separate cassettes (trans) or can be incorporated on the same cassette (cis). Expression of polynucleotides can be driven by the same promoter or by different promoters. It is further recognized that polynucleotides can be stacked at a desired genomic location using a site-specific nuclease or recombination system (e.g., FRT/Flp, Cre/Lox, TALE-endonucleases, zinc finger nucleases, CRISPR/Cas and related technologies). See U.S. Pat. Nos. 7,214,536, 8,921,332, 8,765,448, 5,527,695, 5,744,336, 5,910,415, 6,110,736, 6,175,058, 6,720,475, 6,455,315, 6,458,594 and US Patent Publication Nos. US2019093090, US2019264218, US2018327785, US2017240911, US2016208272, US2019062765.

The expression cassette also can include an additional coding sequence for one or more polypeptides or double stranded RNA molecules (dsRNA) of interest for agronomic traits that primarily are of benefit to a seed company, grower or grain processor. A polypeptide of interest can be any polypeptide encoded by a nucleotide sequence of interest. Non-limiting examples of polypeptides of interest that are suitable for production in plants include those resulting in agronomically important traits such as herbicide resistance (also sometimes referred to as “herbicide tolerance”), virus resistance, bacterial pathogen resistance, insect resistance, nematode resistance, or fungal resistance. See, e.g., U.S. Pat. Nos. 5,569,823; 5,304,730; 5,495,071; 6,329,504; and 6,337,431. The polypeptide also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, etc.). Various polypeptides of interest, as well as methods for introducing these polypeptides into a plant, are described, for example, in U.S. Pat. Nos. 4,761,373; 4,769,061; 4,810,648; 4,940,835; 4,975,374; 5,013,659; 5,162,602; 5,276,268; 5,304,730; 5,495,071; 5,554,798; 5,561,236; 5,569,823; 5,767,366; 5,879,903, 5,928,937; 6,084,155; 6,329,504 and 6,337,431; as well as US Patent Publication No. 2001/0016956.

Polynucleotides conferring resistance/tolerance to an herbicide that inhibits the growing point or meristem, such as an imidazalinone or a sulfonylurea can also be suitable in some embodiments. Exemplary polynucleotides in this category code for mutant ALS and AHAS enzymes as described, e.g., in U.S. Pat. Nos. 5,767,366 and 5,928,937. U.S. Pat. Nos. 4,761,373 and 5,013,659 are directed to plants resistant to various imidazalinone or sulfonamide herbicides. U.S. Pat. No. 4,975,374 relates to plant cells and plants containing a nucleic acid encoding a mutant glutamine synthetase (GS) resistant to inhibition by herbicides that are known to inhibit GS, e.g., phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase (ACCase).

Polypeptides encoded by nucleotides sequences conferring resistance to glyphosate are also suitable for the disclosure. See, e.g., U.S. Pat. Nos. 4,940,835 and 4,769,061. U.S. Pat. No. 5,554,798 discloses transgenic glyphosate resistant maize plants, which resistance is conferred by an altered 5-enolpyruvyl-3-phosphoshikimate (EPSP) synthase gene.

Polynucleotides coding for resistance to phosphono compounds such as glufosinate ammonium or phosphinothricin, and pyridinoxy or phenoxy propionic acids and cyclohexones are also suitable. See, European Patent Application No. 0 242 246. See also, U.S. Pat. Nos. 5,879,903, 5,276,268 and 5,561,236.

Other suitable polynucleotides include those coding for resistance to herbicides that inhibit photosynthesis, such as a triazine and a benzonitrile (nitrilase) See, U.S. Pat. No. 4,810,648. Additional suitable polynucleotides coding for herbicide resistance include those coding for resistance to 2,2-dichloropropionic acid, sethoxydim, haloxyfop, imidazolinone herbicides, sulfonylurea herbicides, triazolopyrimidine herbicides, s-triazine herbicides and bromoxynil. Also suitable are polynucleotides conferring resistance to a protox enzyme, or that provide enhanced resistance to plant diseases; enhanced tolerance of adverse environmental conditions (abiotic stresses) including but not limited to drought, excessive cold, excessive heat, or excessive soil salinity or extreme acidity or alkalinity; and alterations in plant architecture or development, including changes in developmental timing. See, e.g., U.S. Patent Publication No. 2001/0016956 and U.S. Pat. No. 6,084,155.

Additional suitable polynucleotides include those coding for pesticidal (e.g., insecticidal) polypeptides. These polypeptides may be produced in amounts sufficient to control, for example, insect pests (i.e., insect controlling amounts). It is recognized that the amount of production of a pesticidal polypeptide in a plant necessary to control insects or other pests may vary depending upon the cultivar, type of pest, environmental factors and the like. Polynucleotides useful for additional insect or pest resistance include, for example, those that encode toxins identified in Bacillus organisms. Polynucleotides comprising nucleotide sequences encoding Bacillus thuringiensis (Bt) Cry proteins from several subspecies have been cloned and recombinant clones have been found to be toxic to lepidopteran, dipteran and/or coleopteran insect larvae. Examples of such Bt insecticidal proteins include the Cry proteins such as Cry1Aa, Cry1Ab, Cry1Ac, Cry1B, Cry1C, Cry1D, Cry1Ea, Cry1Fa, Cry3A, Cry9A, Cry9B, Cry9C, and the like, as well as vegetative insecticidal proteins such as Vip1, Vip2, Vip3, and the like. A full list of Bt-derived proteins can be found on the worldwide web at Bacillus thuringiensis Toxin Nomenclature Database maintained by the University of Sussex (see also, Crickmore et al. (1998) Microbiol. Mol. Biol. Rev. 62:807-813).

In some embodiments, an additional polypeptide is an insecticidal polypeptide derived from a non-Bt source, including without limitation, an alpha-amylase, a peroxidase, a cholesterol oxidase, a patatin, a protease, a protease inhibitor, a urease, an alpha-amylase inhibitor, a pore-forming protein, a chitinase, a lectin, an engineered antibody or antibody fragment, a Bacillus cereus insecticidal protein, a Xenorhabdus spp. (such as X. nematophila or X. bovienii) insecticidal protein, a Photorhabdus spp. (such as P. luminescens or P. asymobiotica) insecticidal protein, a Brevibacillus spp. (such as B. laterosporous) insecticidal protein, a Lysinibacillus spp. (such as L. sphearicus) insecticidal protein, a Chromobacterium spp. (such as C. subtsugae or C. piscinae) insecticidal protein, a Yersinia spp. (such as Y. entomophaga) insecticidal protein, a Paenibacillus spp. (such as P. propylaea) insecticidal protein, a Clostridium spp. (such as C. bifermentans) insecticidal protein, a Pseudomonas spp. (such as P. fluorescens) and a lignin.

Polypeptides that are suitable for production in plants further include those that improve or otherwise facilitate the conversion of harvested plants or plant parts into a commercially useful product, including, for example, increased or altered carbohydrate content or distribution, improved fermentation properties, increased oil content, increased protein content, improved digestibility, and increased nutraceutical content, e.g., increased phytosterol content, increased tocopherol content, increased stanol content or increased vitamin content. Polypeptides of interest also include, for example, those resulting in or contributing to a reduced content of an unwanted component in a harvested crop, e.g., phytic acid, or sugar degrading enzymes. By “resulting in” or “contributing to” is intended that the polypeptide of interest can directly or indirectly contribute to the existence of a trait of interest (e.g., increasing cellulose degradation by the use of a heterologous cellulase enzyme).

In some embodiments, the polypeptide contributes to improved digestibility for food or feed. Xylanases are hemicellulolytic enzymes that improve the breakdown of plant cell walls, which leads to better utilization of the plant nutrients by an animal. This leads to improved growth rate and feed conversion. Also, the viscosity of the feeds containing xylan can be reduced. Heterologous production of xylanases in plant cells also can facilitate lignocellulosic conversion to fermentable sugars in industrial processing.

Numerous xylanases from fungal and bacterial microorganisms have been identified and characterized (see, e.g., U.S. Pat. No. 5,437,992; Coughlin et al. (1993) “Proceedings of the Second TRICEL Symposium on Trichoderma reesei Cellulases and Other Hydrolases” Espoo; Souminen and Reinikainen, eds. (1993) Foundation for Biotechnical and Industrial Fermentation Research 8:125-135; U.S. Patent Publication No. 2005/0208178; and PCT Publication No. WO 03/16654). In particular, three specific xylanases (XYL-I, XYL-II, and XYL-III) have been identified in T. reesei (Tenkanen et al. (1992) Enzyme Microb. Technol. 14:566; Torronen et al. (1992) Bio/Technology 10:1461; and Xu et al. (1998) Appl. Microbiol. Biotechnol. 49:718).

In other embodiments, a polypeptide useful for the disclosure can be a polysaccharide degrading enzyme. Plants of this disclosure producing such an enzyme may be useful for generating, for example, fermentation feedstocks for bioprocessing. In some embodiments, enzymes useful for a fermentation process include alpha amylases, proteases, pullulanases, isoamylases, cellulases, hemicellulases, xylanases, cyclodextrin glycotransferases, lipases, phytases, laccases, oxidases, esterases, cutinases, granular starch hydrolyzing enzyme and other glucoamylases.

Polysaccharide-degrading enzymes include: starch degrading enzymes such as α-amylases (EC 3.2.1.1), glucuronidases (E.C. 3.2.1.131); exo-1,4-α-D glucanases such as amyloglucosidases and glucoamylase (EC 3.2.1.3), β-amylases (EC 3.2.1.2), α-glucosidases (EC 3.2.1.20), and other exo-amylases; starch debranching enzymes, such as a) isoamylase (EC 3.2.1.68), pullulanase (EC 3.2.1.41), and the like; b) cellulases such as exo-1,4-3-cellobiohydrolase (EC 3.2.1.91), exo-1,3-β-D-glucanase (EC 3.2.1.39), β-glucosidase (EC 3.2.1.21); c) L-arabinases, such as endo-1,5-α-L-arabinase (EC 3.2.1.99), α-arabinosidases (EC 3.2.1.55) and the like; d) galactanases such as endo-1,4-β-D-galactanase (EC 3.2.1.89), endo-1,3-β-D-galactanase (EC 3.2.1.90), α-galactosidase (EC 3.2.1.22), β-galactosidase (EC 3.2.1.23) and the like; e) mannanases, such as endo-1,4-β-D-mannanase (EC 3.2.1.78), 0-mannosidase (EC 3.2.1.25), α-mannosidase (EC 3.2.1.24) and the like; f) xylanases, such as endo-1,4-β-xylanase (EC 3.2.1.8), β-D-xylosidase (EC 3.2.1.37), 1,3-β-D-xylanase, and the like; and g) other enzymes such as α-L-fucosidase (EC 3.2.1.51), α-L-rhamnosidase (EC 3.2.1.40), levanase (EC 3.2.1.65), inulanase (EC 3.2.1.7), and the like. In one embodiment, the α-amylase is the synthetic α-amylase, Amy797E, described is U.S. Pat. No. 8,093,453, herein incorporated by reference in its entirety.

Further enzymes which may be used with the disclosure include proteases, such as fungal and bacterial proteases. Fungal proteases include, but are not limited to, those obtained from Aspergillus, Trichoderma, Mucor and Rhizopus, such as A. niger, A. awamori, A. oryzae and M. miehei. In some embodiments, the polypeptides of this disclosure can be cellobiohydrolase (CBH) enzymes (EC 3.2.1.91). In one embodiment, the cellobiohydrolase enzyme can be CBH1 or CBH2.

Other enzymes useful with the disclosure include, but are not limited to, hemicellulases, such as mannases and arabinofuranosidases (EC 3.2.1.55); ligninases; lipases (e.g., E.C. 3.1.1.3), glucose oxidases, pectinases, xylanases, transglucosidases, alpha 1,6 glucosidases (e.g., E.C. 3.2.1.20); esterases such as ferulic acid esterase (EC 3.1.1.73) and acetyl xylan esterases (EC 3.1.1.72); and cutinases (e.g. E.C. 3.1.1.74).

Double stranded RNA molecules useful with the disclosure include but are not limited to those that suppress target insect genes. As used herein the words “gene suppression”, when taken together, are intended to refer to any of the well-known methods for reducing the levels of protein produced as a result of gene transcription to mRNA and subsequent translation of the mRNA. Gene suppression is also intended to mean the reduction of protein expression from a gene or a coding sequence including posttranscriptional gene suppression and transcriptional suppression. Posttranscriptional gene suppression is mediated by the homology between of all or a part of a mRNA transcribed from a gene or coding sequence targeted for suppression and the corresponding double stranded RNA used for suppression, and refers to the substantial and measurable reduction of the amount of available mRNA available in the cell for binding by ribosomes. The transcribed RNA can be in the sense orientation to effect what is called co-suppression, in the anti-sense orientation to effect what is called anti-sense suppression, or in both orientations producing a dsRNA to effect what is called RNA interference (RNAi). Transcriptional suppression is mediated by the presence in the cell of a dsRNA, a gene suppression agent, exhibiting substantial sequence identity to a promoter DNA sequence or the complement thereof to effect what is referred to as promoter trans suppression. Gene suppression may be effective against a native plant gene associated with a trait, e.g., to provide plants with reduced levels of a protein encoded by the native gene or with enhanced or reduced levels of an affected metabolite. Gene suppression can also be effective against target genes in plant pests that may ingest or contact plant material containing gene suppression agents, specifically designed to inhibit or suppress the expression of one or more homologous or complementary sequences in the cells of the pest. Such genes targeted for suppression can encode an essential protein, the predicted function of which is selected from the group consisting of muscle formation, juvenile hormone formation, juvenile hormone regulation, ion regulation and transport, digestive enzyme synthesis, maintenance of cell membrane potential, amino acid biosynthesis, amino acid degradation, sperm formation, pheromone synthesis, pheromone sensing, antennae formation, wing formation, leg formation, development and differentiation, egg formation, larval maturation, digestive enzyme formation, hemolymph synthesis, hemolymph maintenance, neurotransmission, cell division, energy metabolism, respiration, and apoptosis.

Transgenic Cells, Plants, Plant parts, Seed

In some aspects, the disclosure further provides transgenic cells, plants, plant parts, and seed comprising the insecticidal proteins or nucleic acids of the disclosure. In some embodiments, the disclosure provides a non-human host cell comprising a polynucleotide, a nucleic acid molecule, an expression cassette, a vector, or a polypeptide of the disclosure. The transgenic non-human host cell can include, but is not limited to, a plant cell (including a monocot cell and/or a dicot cell), a yeast cell, a bacterial cell or an insect cell. Accordingly, in some embodiments, a bacterial cell is provided which is selected from the genera Bacillus, Brevibacillus, Clostridium, Xenorhabdus, Photorhabdus, Pasteuria, Escherichia, Pseudomonas, Erwinia, Serratia, Klebsiella, Salmonella, Pasteurella, Xanthomonas, Streptomyces, Rhizobium, Rhodopseudomonas, Methylophilius, Agrobacterium, Acetobacter, Lactobacillus, Arthrobacter, Azotobacter, Leuconostoc, or Alcaligenes. Thus, for example, as biological insect control agents, the disclosed insecticidal proteins can be produced by expression of a polynucleotide encoding the same in a bacterial cell. For example, in some embodiments, a Bacillus thuringiensis cell comprising a polynucleotide encoding an insecticidal protein of the disclosure is provided.

In some embodiments, the transgenic plant cell is a dicot plant cell or a monocot plant cell. In additional embodiments, the dicot plant cell is a soybean cell, sunflower cell, tomato cell, cole crop cell, cotton cell, sugar beet cell or a tobacco cell. In further embodiments, the monocot cell is a barley cell, maize cell, oat cell, rice cell, sorghum cell, sugar cane cell or wheat cell. In some embodiments, the disclosure provides a plurality of dicot cells or monocot cells comprising a polynucleotide expressing a disclosed insecticidal protein. In some embodiments, the plurality of cells are juxtaposed to form an apoplast and are grown in natural sunlight. In some embodiments, the transgenic plant cell cannot regenerate a whole plant.

In other embodiments of the disclosure, an insecticidal protein of the disclosure is expressed in a higher organism, for example, a plant. Such transgenic plants expressing effective amounts of the insecticidal protein to control plant pests such as insect pests. When an insect starts feeding on such a transgenic plant, it ingests the expressed insecticidal protein. This can deter the insect from further biting into the plant tissue or may even harm or kill the insect. In some embodiments, a disclosed polynucleotide is inserted into an expression cassette, which is then stably integrated in the genome of the plant. In other embodiments, the polynucleotide is included in a non-pathogenic self-replicating virus.

In some embodiments of the disclosure, a transgenic plant cell comprising a nucleic acid molecule or polypeptide of the disclosure is a cell of a plant part, a plant organ or a plant culture (each as described herein) including, but not limited to, a root, a leaf, a seed, a flower, a fruit, a pollen cell, organ or plant culture, and the like, or a callus cell or culture.

A transgenic plant or plant cell transformed in accordance with the disclosure may be a monocot or dicot plant or plant cell and includes, but is not limited to, corn (maize), soybean, rice, wheat, barley, rye, oats, sorghum, millet, sunflower, safflower, sugar beet, cotton, sugarcane, oilseed rape, alfalfa, tobacco, peanuts, vegetables, including, sweet potato, bean, pea, chicory, lettuce, cabbage, cauliflower, broccoli, turnip, carrot, eggplant, cucumber, radish, spinach, potato, tomato, asparagus, onion, garlic, melons, pepper, celery, squash, pumpkin, zucchini, fruits, including, apple, pear, quince, plum, cherry, peach, nectarine, apricot, strawberry, grape, raspberry, blackberry, pineapple, avocado, papaya, mango, banana, and specialty plants, such as Arabidopsis, and woody plants such as coniferous and deciduous trees. Preferably, plants of the of the disclosure are crop plants such as maize, sorghum, wheat, sunflower, tomato, crucifers, peppers, potato, cotton, rice, soybean, sugar beet, sugarcane, tobacco, barley, oilseed rape, and the like.

Once a desired polynucleotide has been transformed into a particular plant species, it may be propagated in that species or moved into other varieties of the same species, particularly including commercial varieties, using any appropriate technique including traditional breeding techniques.

The disclosed insecticidal proteins can function in the plant part, plant cell, plant organ, seed, harvested product, processed product or extract, and the like, as an insect control agent. In other words, in some embodiments the insecticidal proteins can continue to perform the insecticidal function it had in the transgenic plant. The nucleic acid can function to express the insecticidal protein. As an alternative to encoding the insecticidal protein of the disclosure, the nucleic acid can function to identify a transgenic plant part, plant cell, plant organ, seed, harvested product, processed product or extract of the disclosure.

In some embodiments, a transgenic plant, plant part, plant cell, plant organ, or seed of the disclosure is hemizygous for a polynucleotide or expression cassette of the disclosure, e.g., a hybrid maize plant where only one parent plant contained the polynucleotide or expression cassette of the disclosure. In some embodiments, a transgenic plant, plant part, plant cell, plant organ, or seed of the disclosure is homozygous for a polynucleotide or expression cassette of the disclosure.

Additional embodiments of the disclosure include harvested products produced from the transgenic plants or parts thereof of the disclosure, as well as a processed product produced from the harvested products. A harvested product can be a whole plant or any plant part, as described herein. Thus, in some embodiments, non-limiting examples of a harvested product include a seed, a fruit, a flower or part thereof (e.g., an anther, a stigma, and the like), a leaf, a stem, and the like. In other embodiments, a processed product includes, but is not limited to, a flour, meal, oil, starch, cereal, and the like produced from a harvested seed or other plant part of the disclosure, wherein said seed or other plant part comprises a nucleic acid molecule/polynucleotide/nucleotide sequence of this disclosure.

In other embodiments, the disclosure provides an extract from a transgenic seed or a transgenic plant of the disclosure, wherein the extract comprises a nucleic acid molecule, a polynucleotide, a nucleotide sequence or an insecticidal protein of the disclosure. Extracts from plants or plant parts can be made according to procedures well known in the art (See, de la Torre et al., Food, Agric. Environ. 2(1):84-89 (2004); Guidet, Nucleic Acids Res. 22(9): 1772-1773 (1994); Lipton et al., Food Agric. Immun. 12:153-164 (2000)). Such extracts may be used, e.g., in methods to detect the presence of an insecticidal protein or a polynucleotide of the disclosure.

In some embodiments, a transgenic plant, plant part, plant cell, plant organ, seed, harvested product, processed product or extract has increased insecticidal activity to one or more insect pests (e.g., a coleopteran pest, such as Western corn rootworm) as compared with a suitable control that does not comprise a nucleic acid encoding an insecticidal protein of the disclosure.

### Plant Transformation

Non-limiting examples of methods for transformation of plants include transformation via bacterial-mediated nucleic acid delivery (e.g., via Agrobacterium), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

For Agrobacterium-mediated transformation, binary vectors or vectors carrying at least one T-DNA border sequence are generally suitable, whereas for direct gene transfer (e.g., particle bombardment and the like) any vector is suitable and linear DNA containing only the construction of interest can be used. In the case of direct gene transfer, transformation with a single DNA species or co-transformation can be used (Schocher et al., Biotechnology 4:1093-1096 (1986)). For both direct gene transfer and Agrobacterium-mediated transfer, transformation is usually (but not necessarily) undertaken with a selectable marker that may be a positive selection (e.g., Phosphomannose Isomerase), provide resistance to an antibiotic (e.g., kanamycin, hygromycin or methotrexate) or a herbicide (e.g., glyphosate or glufosinate). However, the choice of selectable marker is not critical to the disclosure.

Agrobacterium-mediated transformation is a commonly used method for transforming plants because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. (1993) Plant Cell 5:159-169). The transfer of the recombinant binary vector to Agrobacterium can be accomplished by a triparental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Hofgen & Willmitzer (1988) Nucleic Acids Res. 16:9877).

Dicots as well as monocots may be transformed using Agrobacterium. Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBini9 (Bevan et al., Nucl. Acids Res. 12 (1984) 8711). Agrobacteria transformed by such a vector can then be used in known manner for the transformation of plants, such as plants used as a model, like Arabidopsis or crop plants such as, by way of example, tobacco plants, for example by immersing bruised leaves or chopped leaves in an Agrobacterial solution and then culturing them in suitable media. The transformation of plants by means of Agrobacterium tumefaciens is described, for example, by Hagen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

Soybean plant material can be suitably transformed, and fertile plants regenerated by many methods which are well known to one of skill in the art. For example, fertile morphologically normal transgenic soybean plants may be obtained by: 1) production of somatic embryogenic tissue from, e.g., immature cotyledon, hypocotyl or other suitable tissue; 2) transformation by particle bombardment or infection with Agrobacterium; and 3) regeneration of plants. In one example, as described in U.S. Pat. No. 5,024,944, cotyledon tissue is excised from immature embryos of soybean, preferably with the embryonic axis removed, and cultured on hormone-containing medium to form somatic embryogenic plant material. This material is transformed using, for example, direct DNA methods, DNA coated microprojectile bombardment or infection with Agrobacterium, cultured on a suitable selection medium and regenerated, optionally also in the continued presence of selecting agent, into fertile transgenic soybean plants. Selection agents may be antibiotics such as kanamycin, hygromycin, or herbicides such as phosphinothricin or glyphosate or, alternatively, selection may be based upon expression of a visualizable marker gene such as GUS. Alternatively, target tissues for transformation comprise meristematic rather than somaclonal embryogenic tissue or, optionally, is flower or flower-forming tissue. Other examples of soybean transformations can be found, e.g. by physical DNA delivery method, such as particle bombardment (Finer and McMullen (1991) In Vitro Cell Dev. Biol. 27P:175-182; McCabe et al. (1988) Bio/technology 6:923-926), whisker (Khalafalla et al. (2006) African J. of Biotechnology 5:1594-1599), aerosol bean injection (U.S. Pat. No. 7,001,754), or by Agrobacterium-mediated delivery methods (Hinchee et al. (1988) Bio/Technology 6:915-922; U.S. Pat. No. 7,002,058; U.S. Patent Application Publication No. 20040034889; U.S. Patent Application Publication No. 20080229447; Paz et al. (2006) Plant Cell Report 25:206-213).

Soybean transgenic plants can be generated with the heretofore described binary vectors containing selectable marker genes with different transformation methods. For example, a vector is used to transform immature seed targets as described (see e.g., U.S. Patent Application Publication No. 20080229447) to generate transgenic HPPD soybean plants directly using HPPD inhibitor, such as mesotrione, as selection agent. Optionally, other herbicide tolerance genes can be present in the polynucleotide alongside other sequences which provide additional means of selection/identification of transformed tissue including, for example, the known genes which provide resistance to kanamycin, hygromycin, phosphinothricin, butafenacil, or glyphosate. For example, different binary vectors containing PAT or EPSPS selectable marker genes are transformed into immature soybean seed target to generate pesticidal and herbicide tolerant plants using Agrobacterium-mediated transformation and glufosinate or glyphosate selection as described (see e.g., U.S. Patent Application Publication No. 20080229447).

Transformation of a plant by recombinant Agrobacterium usually involves co-cultivation of the Agrobacterium with explants from the plant and follows methods well known in the art. Transformed tissue is regenerated on selection medium carrying an antibiotic or herbicide resistance marker between the binary plasmid T-DNA borders.

As discussed previously, another method for transforming plants, plant parts and plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., a dried yeast cell, a dried bacterium or a bacteriophage, each containing one or more nucleic acids sought to be introduced) also can be propelled into plant tissue.

In other embodiments, a polynucleotide of the disclosure can be directly transformed into the plastid genome. Plastid transformation technology is extensively described in U.S. Pat. Nos. 5,451,513, 5,545,817, and 5,545,818, in PCT application no. WO 95/16783, and in McBride et al. (1994) Proc. Nati. Acad. Sci. USA 91, 7301-7305.

Methods of selecting for transformed, transgenic plants, plant cells or plant tissue culture can be employed in the methods of the disclosure provided herein. For example, a recombinant vector of the disclosure also can include an expression cassette comprising a nucleotide sequence for a selectable marker, which can be used to select a transformed plant, plant part or plant cell.

Examples of selectable markers include, but are not limited to, a nucleotide sequence encoding neo or nptII, which confers resistance to kanamycin, G418, and the like (Potrykus et al. (1985) Mol. Gen. Genet. 199:183-188); a nucleotide sequence encoding bar, which confers resistance to phosphinothricin; a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaenae that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J. Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; or a nucleotide sequence encoding hph that confers resistance to hygromycin. One of skill in the art is capable of choosing a suitable selectable marker for use in an expression cassette of this disclosure.

Additional selectable markers include, but are not limited to, a nucleotide sequence encoding β-glucuronidase or uidA (GUS) that encodes an enzyme for which various chromogenic substrates are known; an R-locus nucleotide sequence that encodes a product that regulates the production of anthocyanin pigments (red color) in plant tissues (Dellaporta et al., “Molecular cloning of the maize R-nj allele by transposon-tagging with Ac” 263-282 In: Chromosome Structure and Function: Impact of New Concepts, 18th Stadler Genetics Symposium (Gustafson & Appels eds., Plenum Press 1988)); a nucleotide sequence encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleotide sequence encoding xylE that encodes a catechol dioxygenase (Zukowsky et al. (1983) Proc. Natl. Acad. Sci. USA 80:1101-1105); a nucleotide sequence encoding tyrosinase, an enzyme capable of oxidizing tyrosine to DOPA and dopaquinone, which in turn condenses to form melanin (Katz et al. (1983) J. Gen. Microbiol. 129:2703-2714); a nucleotide sequence encoding β-galactosidase, an enzyme for which there are chromogenic substrates; a nucleotide sequence encoding luciferase (lux) that allows for bioluminescence detection (Ow et al. (1986) Science 234:856-859); a nucleotide sequence encoding aequorin which may be employed in calcium-sensitive bioluminescence detection (Prasher et al. (1985) Biochem. Biophys. Res. Comm. 126:1259-1268); or a nucleotide sequence encoding green fluorescent protein (Niedz et al. (1995) Plant Cell Reports 14:403-406) or other fluorescent protein such as dsRed or mCherry. One of skill in the art is capable of choosing a suitable selectable marker for use in an expression cassette of this disclosure.

Further, as is well known in the art, intact transgenic plants can be regenerated from transformed plant cells, plant tissue culture or cultured protoplasts using any of a variety of known techniques. Plant regeneration from plant cells, plant tissue culture or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)).

Additionally, the genetic properties engineered into the transgenic seeds and plants, plant parts, or plant cells of the disclosure described above can be passed on by sexual reproduction or vegetative growth and therefore can be maintained and propagated in progeny plants. Generally, maintenance and propagation make use of known agricultural methods developed to fit specific purposes such as harvesting, sowing or tilling.

A polynucleotide therefore can be introduced into the plant, plant part or plant cell in any number of ways that are well known in the art, as described above. Therefore, no particular method for introducing one or more polynucleotides into a plant is relied upon, rather any method that allows the one or more polynucleotides to be stably integrated into the genome of the plant can be used. Where more than one polynucleotide is to be introduced, the respective polynucleotides can be assembled as part of a single nucleic acid molecule, or as separate nucleic acid molecules, and can be located on the same or different nucleic acid molecules. Accordingly, the polynucleotides can be introduced into the cell of interest in a single transformation event, in separate transformation events, or, for example, in plants, as part of a breeding protocol.

Once a desired polynucleotide has been transformed into a particular plant species, it may be propagated in that species or moved into other varieties of the same species, particularly including commercial varieties, using traditional breeding techniques.

### Insecticidal Compositions

In some embodiments, an insecticidal composition is provided comprising an insecticidal protein of the disclosure in an agriculturally acceptable carrier. As used herein an “agriculturally-acceptable carrier” can include natural or synthetic, organic or inorganic material which is combined with the active protein to facilitate its application to or in the plant, or part thereof. Examples of agriculturally acceptable carriers include, without limitation, powders, dusts, pellets, granules, sprays, emulsions, colloids, and solutions. Agriculturally-acceptable carriers further include, but are not limited to, inert components, dispersants, surfactants, adjuvants, tackifiers, stickers, binders, or combinations thereof, that can be used in agricultural formulations. Such compositions can be applied in any manner that brings the pesticidal proteins or other pest control agents in contact with the pests. Accordingly, the compositions can be applied to the surfaces of plants or plant parts, including seeds, leaves, flowers, stems, tubers, roots, and the like. In other embodiments, a plant producing an insecticidal protein of the disclosure in planta is an agriculturally-acceptable carrier of the expressed insecticidal protein, the combination of plant and the protein is an insecticidal composition.

In further embodiments, the insecticidal composition comprises a bacterial cell or a transgenic bacterial cell of the disclosure, wherein the bacterial cell or transgenic bacterial cell produces an insecticidal protein of the disclosure. Such an insecticidal composition can be prepared by desiccation, lyophilization, homogenization, extraction, filtration, centrifugation, sedimentation, or concentration of a culture of Bacillus thuringiensis (Bt), including a transgenic Bt culture. In some embodiments, a composition of the disclosure may comprise at least about 1%, at least about 5%, at least about 10%, at least about 20%, at least about 25%, at least about 30%, at least about 35%, at least about 40%, at least about 50%, at least about 60%, at least about 70%, at least about 80%, at least about 90%, at least about 95%, at least about 97%, or at least 99% by weight a polypeptide of the disclosure. In additional embodiments, the composition comprises from about 1% to about 99% by weight of the insecticidal protein of the disclosure.

Disclosed insecticidal proteins can be used in combination with other pest control agents to increase pest target spectrum and/or for the prevention or management of insect resistance. Furthermore, the use of the disclosed insecticidal proteins in combination with an insecticidal agent which has a different mode of action or target a different receptor in the insect gut has particular utility for the prevention and/or management of insect resistance.

Therefore, in some embodiments, a composition is provided that controls one or more plant pests (e.g., an insect pest such as a lepidopteran insect pest, a coleopteran insect pest, a hemipteran insect pest and/or a dipteran insect pest), wherein the composition comprises a first pest control agent, which is a disclosed insecticidal protein and at least a second pest control agent that is different from the first pest control agent. In other embodiments, the composition is a formulation for topical application to a plant. In still other embodiments, the composition is a transgenic plant. In further embodiments, the composition is a combination of a formulation topically applied to a transgenic plant. In some embodiments, the formulation comprises the first pest control agent, which is a disclosed insecticidal protein when the transgenic plant comprises the second pest control agent. In other embodiments, the formulation comprises the second pest control agent when the transgenic plant comprises the first pest control agent, which is an insecticidal protein of the disclosure.

In some embodiments, the second pest control agent can be one or more of a chemical pesticide, such as an insecticide, a Bacillus thuringiensis (Bt) insecticidal protein, and/or a non-Bt pesticidal agent including without limitation a Xenorhabdus insecticidal protein, a Photorhabdus insecticidal protein, a Brevibacillus laterosporus insecticidal protein, a Bacillus sphaericus insecticidal protein, a protease inhibitor (both serine and cysteine types), a lectin, an alpha-amylase, a peroxidase, a cholesterol oxidase, or a double stranded RNA (dsRNA) molecule.

In other embodiments, the second pest control agent is one or more chemical pesticides, which is optionally a seed coating. Non-limiting examples of chemical pesticides include pyrethroids, carbamates, neonicotinoids, neuronal sodium channel blockers, insecticidal macrocyclic lactones, gamma-aminobutyric acid (GABA) antagonists, insecticidal ureas and juvenile hormone mimics. In other embodiments, the chemical pesticide is one or more of abamectin, acephate, acetamiprid, amidoflumet (S-1955), avermectin, azadirachtin, azinphos-methyl, bifenthrin, binfenazate, buprofezin, carbofuran, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, diflubenzuron, dimethoate, diofenolan, emamectin, endosulfan, esfenvalerate, ethiprole, fenothicarb, fenoxycarb, fenpropathrin, fenproximate, fenvalerate, fipronil, flonicamid, flucythrinate, tau-fluvalinate, flufenerim (UR-50701), flufenoxuron, fonophos, halofenozide, hexaflumuron, imidacloprid, indoxacarb, isofenphos, lufenuron, malathion, metaldehyde, methamidophos, methidathion, methomyl, methoprene, methoxychlor, monocrotophos, methoxyfenozide, nithiazin, novaluron, noviflumuron (XDE-007), oxamyl, parathion, parathion-methyl, permethrin, phorate, phosalone, phosmet, phosphamidon, pirimicarb, profenofos, pymetrozine, pyridalyl, pyriproxyfen, rotenone, spinosad, spiromesifin (BSN 2060), sulprofos, tebufenozide, teflubenzuron, tefluthrin, terbufos, tetrachlorvinphos, thiacloprid, thiamethoxam, thiodicarb, thiosultap-sodium, tralomethrin, trichlorfon and triflumuron, aldicarb, oxamyl, fenamiphos, amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad. In still other embodiments, the chemical pesticide is selected from one or more of cypermethrin, cyhalothrin, cyfluthrin and beta-cyfluthrin, esfenvalerate, fenvalerate, tralomethrin, fenothicarb, methomyl, oxamyl, thiodicarb, clothianidin, imidacloprid, thiacloprid, indoxacarb, spinosad, abamectin, avermectin, emamectin, endosulfan, ethiprole, fipronil, flufenoxuron, triflumuron, diofenolan, pyriproxyfen, pymetrozine and amitraz.

In additional embodiments, the second pest control agent can be one or more of any number of Bacillus thuringiensis insecticidal proteins including but not limited to a Cry protein, a vegetative insecticidal protein (VIP) and insecticidal chimeras of any of the preceding insecticidal proteins. In other embodiments, the second pest control agent is a Cry protein selected from: Cry1Aa, Cry1Ab, Cry1Ac, Cry1Ad, Cry1Ae, Cry1Af, Cry1Ag, Cry1Ah, Cry1Ai, Cry1Aj, Cry1Ba, Cry1Bb, Cry1Bc, Cry1Bd, Cry1Be, Cry1Bf, Cry1Bg, Cry1Bh, Cry1Bi, Cry1Ca, Cry1Cb, Cry1 Da, Cry1Db, Cry1Dc, Cry1Dd, Cry1Ea, Cry1Eb, Cry1Fa, Cry1Fb, Cry1Ga, Cry1Gb, Cry1Gc, Cry1Ha, Cry1Hb, Cry1Hc, Cry1Ia, Cry1Ib, Cry1Ic, Cry1Id, Cry1Ie, Cry1If, Cry1Ig, Cry1Ja, Cry1Jb, Cry1Jc, Cry1Jd, Cry1Ka, Cry1La, Cry1Ma, Cry1Na, Cry1Nb, Cry2Aa, Cry2Ab, Cry2Ac, Cry2Ad, Cry2Ae, Cry2Af, Cry2Ag, Cry2Ah, Cry2Ai, Cry2Aj, Cry2Ak, Cry2Al, Cry2Ba, Cry3Aa, Cry3Ba, Cry3Bb, Cry3Ca, Cry4Aa, Cry4Ba, Cry4Ca, Cry4Cb, Cry4Cc, Cry5Aa, Cry5Ab, Cry5Ac, Cry5Ad, Cry5Ba, Cry5Ca, Cry5 Da, Cry5Ea, Cry6Aa, Cry6Ba, Cry7Aa, Cry7Ab, Cry7Ac, Cry7Ba, Cry7Bb, Cry7Ca, Cry7Cb, Cry7 Da, Cry7Ea, Cry7Fa, Cry7Fb, Cry7Ga, Cry7Gb, Cry7Gc, Cry7Gd, Cry7Ha, Cry7Ia, Cry7Ja, Cry7Ka, Cry7Kb, Cry7La, Cry8Aa, Cry8Ab, Cry8Ac, Cry8Ad, Cry8Ba, Cry8Bb, Cry8Bc, Cry8Ca, Cry8 Da, Cry8Db, Cry8Ea, Cry8Fa, Cry8Ga, Cry8Ha, Cry8Ia, Cry8Ib, Cry8Ja, Cry8Ka, Cry8Kb, Cry8La, Cry8Ma, Cry8Na, Cry8 Pa, Cry8Qa, Cry8Ra, Cry8Sa, Cry8Ta, Cry9Aa, Cry9Ba, Cry9Bb, Cry9Ca, Cry9 Da, Cry9Db, Cry9Dc, Cry9Ea, Cry9Eb, Cry9Ec, Cry9Ed, Cry9Ee, Cry9Fa, Cry9Ga, Cry10Aa, Cry11Aa, Cry11Ba, Cry11Bb, Cry12Aa, Cry13Aa, Cry14Aa, Cry14Ab, Cry15Aa, Cry16Aa, Cry17Aa, Cry18Aa, Cry18Ba, Cry18Ca, Cry19Aa, Cry19Ba, Cry19Ca, Cry20Aa, Cry20Ba, Cry21Aa, Cry21Ba, Cry21Ca, Cry21 Da, Cry21Ea, Cry21Fa, Cry21Ga, Cry21Ha, Cry22Aa, Cry22Ab, Cry22Ba, Cry22Bb, Cry23Aa, Cry24Aa, Cry24Ba, Cry24Ca, Cry25Aa, Cry26Aa, Cry27Aa, Cry28Aa, Cry29Aa, Cry29Ba, Cry30Aa, Cry30Ba, Cry30Ca, Cry30 Da, Cry30Db, Cry30Ea, Cry30Fa, Cry30Ga, Cry31Aa, Cry31Ab, Cry31Ac, Cry31Ad, Cry32Aa, Cry32Ab, Cry32Ba, Cry32Ca, Cry32Cb, Cry32 Da, Cry32Ea, Cry32Eb, Cry32Fa, Cry32Ga, Cry32Ha, Cry32Hb, Cry32Ia, Cry32Ja, Cry32Ka, Cry32La, Cry32Ma, Cry32 Mb, Cry32Na, Cry32Oa, Cry32 Pa, Cry32Qa, Cry32Ra, Cry32Sa, Cry32Ta, Cry32Ua, Cry33Aa, Cry34Aa, Cry34Ab, Cry34Ac, Cry34Ba, Cry35Aa, Cry35Ab, Cry35Ac, Cry35Ba, Cry36Aa, Cry37Aa, Cry38Aa, Cry39Aa, Cry40Aa, Cry40Ba, Cry40Ca, Cry40 Da, Cry41Aa, Cry41Ab, Cry41Ba, Cry42Aa, Cry43Aa, Cry43Ba, Cry43Ca, Cry43Cb, Cry43Cc, Cry44Aa, Cry45Aa, Cry46Aa Cry46Ab, Cry47Aa, Cry48Aa, Cry48Ab, Cry49Aa, Cry49Ab, Cry50Aa, Cry50Ba, Cry51Aa, Cry52Aa, Cry52Ba, Cry53Aa, Cry53Ab, Cry54Aa, Cry54Ab, Cry54Ba, Cry55Aa, Cry56Aa, Cry57Aa, Cry57Ab, Cry58Aa, Cry59Aa, Cry59Ba, Cry60Aa, Cry60Ba, Cry61Aa, Cry62Aa, Cry63Aa, Cry64Aa, Cry65Aa, Cry66Aa, Cry67Aa, Cry68Aa, Cry69Aa, Cry69Ab, Cry70Aa, Cry70Ba, Cry70Bb, Cry71Aa, Cry72Aa, Cry73Aa, or any combination of the foregoing. In some embodiments, the second pest control agent comprises the Cry1Ab protein in the Bt 11 event (see U.S. Pat. No. 6,114,608), the Cry3A055 protein in the MIR604 event (see U.S. Pat. No. 8,884,102), the eCry3.1Ab protein in the 5307 event (see US Patent No. U.S. Ser. No. 10/428,393) and/or the mCry3A protein in the MZI098 event (see US Patent Application No. US20200190533). In some embodiments, the second pest control agent comprises the Bt 11 event (see U.S. Pat. No. 6,114,608), the MIR604 event (see U.S. Pat. No. 8,884,102), the 5307 event (see US Patent No. U.S. Ser. No. 10/428,393) and/or the MZI098 event (see US Patent Application No. US20200190533).

In further embodiments, the second pest control agent is one or more Vip3 vegetative insecticidal proteins. Some structural features that identify a protein as being in the Vip3 class of proteins includes: 1) a size of about 80-88 kDa that is proteolytically processed by insects or trypsin to about a 62-66 kDa toxic core (Lee et al. 2003. Appl. Environ. Microbiol. 69:4648-4657); and 2) a highly conserved N-terminal secretion signal which is not naturally processed during secretion in B. thuringiensis. Non-limiting examples of members of the Vip3 class and their respective GenBank accession numbers, U.S. Patent or patent publication number are Vip3Aa1 (AAC37036), Vip3Aa2 (AAC37037), Vip3Aa3 (U.S. Pat. No. 6,137,033), Vip3Aa4 (AAR81079), Vip3Aa5 (AAR81080), Vip3Aa6 (AAR81081), Vip3Aa7 (AAK95326), Vip3Aa8 (AAK97481), Vip3Aa9 (CAA76665), Vip3Aa10 (AAN60738), Vip3Aa11 (AAR36859), Vip3Aa12 (AAM22456), Vip3Aa13 (AAL69542), Vip3Aa14 (AAQ12340), Vip3Aa15 (AAP51131), Vip3Aa16 (AAW65132), Vip3Aa17 (U.S. Pat. No. 6,603,063), Vip3Aa18 (AAX49395), Vip3Aa19 (DQ241674), Vip3Aa19 (DQ539887), Vip3Aa20 (DQ539888), Vip3Aa21 (ABD84410), Vip3Aa22 (AAY41427), Vip3Aa23 (AAY41428), Vip3Aa24 (BI 880913), Vip3Aa25 (EF608501), Vip3Aa26 (EU294496), Vip3Aa27 (EU332167), Vip3Aa28 (FJ494817), Vip3Aa29 (FJ626674), Vip3Aa30 (FJ626675), Vip3Aa31 (FJ626676), Vip3Aa32 (FJ626677), Vip3Aa33 (GU073128), Vip3Aa34 (GU073129), Vip3Aa35 (GU733921), Vip3Aa36 (GU951510), Vip3Aa37 (HM132041), Vip3Aa38 (HM117632), Vip3Aa39 (HMi17631), Vip3Aa40 (HM132042), Vip3Aa41 (HM132043), Vip3Aa42 (HQ587048), Vip3Aa43 (HQ594534), Vip3Aa44 (HQ650163), Vip3Ab1 (AAR40284), Vip3Ab2 (AAY88247), Vip3Ac1 (U.S. Patent Application Publication 20040128716), Vip3Ad1 (U.S. Patent Application Publication 20040128716), Vip3Ad2 (CAI43276), Vip3Ae1 (CAI43277), Vip3Af1 (U.S. Pat. No. 7,378,493), Vip3Af2 (ADN08753), Vip3Af3 (HMi17634), Vip3Ag1 (ADN08758), Vip3Ag2 (FJ556803), Vip3Ag3 (HM117633), Vip3Ag4 (HQ414237), Vip3Ag5 (HQ542193), Vip3Ah1 (DQ832323), Vip3Ba1 (AAV70653), Vip3Ba2 (HM117635), Vip3Bb1 (U.S. Pat. No. 7,378,493), Vip3Bb2 (AB030520) and Vip3Bb3 (ADI48120). In some embodiments, the Vip3 protein is Vip3Aa (U.S. Pat. No. 6,137,033), for example, as represented by corn event MIR162 (U.S. Pat. Nos. 8,232,456; 8,455,720; and 8,618,272). In some embodiments, the second pest control agent comprises the event MIR162 (U.S. Pat. Nos. 8,232,456; 8,455,720; and 8,618,272).

In some embodiments, the second pest control agent comprises any one or more of the insecticidal proteins or dsRNAs present in any of the following events: the Bt11 event (see U.S. Pat. No. 6,114,608), the MIR604 event (see U.S. Pat. No. 8,884,102), the MIR162 event (see U.S. Pat. No. 8,232,456), the 5307 event (see US Patent No. U.S. Ser. No. 10/428,393), the MZIR098 event (see US Patent Application No. US20200190533), the TC1507 event (see U.S. Pat. No. 7,288,643), the DAS-59122-7 event (see U.S. Pat. No. 7,323,556), the MON810 event (see U.S. Pat. No. 6,713,259), the MON863 event (see U.S. Pat. No. 7,705,216), the MON89034 event (see U.S. Pat. No. 8,062,840), the NK603 event (see U.S. Pat. No. 6,825,400), the MON88017 event (see U.S. Pat. No. 9,556,492), the DP-4114 event (see U.S. Pat. No. 9,725,772), the MON87411 event (see U.S. Pat. No. 9,441,240), the DAS40278 event (see PCT publication No. WO11/022469), the T25 event, the DP-032218-9 event (see US Patent Application No. US2015361447), the DP-033121-3 event (see US Patent Application No. US2015361446), the DP-023211-2 event (see PCT Publication No. WO2019209700), the DP-202216-6 event (see PCT Publication No. WO19/204253), the DP-051292-2 event (see PCT Publication WO24/112634), the MON95379 event (see US Patent Application No. US2020032289), the MON87429 event (see PCT Publication No. WO19/152316), the ZM_CSM36715 event (see PCT Publication No. WO2024/137408), the DBN9936 event (see PCT Publication No. WO2016173361), the DBN9501 event (see PCT Publication No. WO20207125), the DBN9858 event (see PCT Publication No. WO16/173508) the Origin GH5112E-117C event (see PCT Publication No. WO17/088480), the LP007-1 event (see Chinese Patent Application No. CN112852801), the LP007-2 event (see Chinese Patent Application No. CN112831584), the LP007-3 event (see Chinese Patent Application No. CN112877454), the LP007-4 event (see Chinese Patent Application No. CN112831585), the LP007-5 event (see Chinese Patent Application No. CN113151534), the LP007-6 event (see Chinese Patent Application No. CN113151533), the LP007-7 event (see Chinese Patent Application No. CN112852991), the LP007-8 event (see CN113980958), Ruifeng8, ND207, or the Ruifeng125 event (see Chinese Patent Application No. CN105017391), the KJ1172 event (see Chinese Patent Application No. CN1164109774), the LD02 event (see Chinese Patent Application No. CN115820630A), the LD05 event (See Chinese Patent Application No. CN116287384), the LG11 event (see Chinese Patent Application Nos. CN116200529A and CN115725571A), the DBN9235 event (see Chinese Patent Application No. CN116219063), the DBN9508 event (see Chinese Patent No. CN109971880), the DBN9888 event (see PCT Publication No. WO16/173540), the DBN9978 event (see PCT Publication No. WO16/1733 62), the DBN9953 event (see Chinese Patent No. CN104878092), the DBN9927 event (see PCT Publication No. WO16/173360), the LP026-1 event (see Chinese Application No. CN116144672A), the LP026-2 event (see Chinese Application No. CN116144818A), the LP026-3 event (see Chinese Application No. CN116144671A), the LP026-4 event (see Chinese Application No. CN116144817A), the LP026-5 event (see Chinese Application No. CN116200519A), the 2A-7 event (see Chinese Patent Application No. CN112280743A), the CA09328 event (see Chinese Patent Application No. CN112126707A), the ZM8-143 event (see Chinese Patent Application No. CN108018286), the ZM1-027 event (see Chinese Patent Application No. CN108018368A), the DP-915635-4 event (see PCT Publication No. WO21/247204), the DP-910521-2 event (see PCT Publication No. WO23/091888), the DAS-01131-3 event (see PCT Publication No. WO23/091884), the MON95275event (see PCT Publication No. WO21/216571), the ME240913 event (see PCT Publication No. WO21/087586), the MZIR260 event (see PCT application No. PCT/US2025/018666) the EH913 event (see PCT Publication No. WO21/087586), the GTS 40-3-2 event, the MON87701 event (see PCT Publication No. WO09/64652), the MON87708 event (see PCT Publication No. WO11/034704), the MON87751 event (see PCT Publication No. WO14/201235), the MON98788 event (see PCT Publication No. WO06/128095), the A5547-127/A2704-12 event (see PCT Publications Nos. 006/108674 and WO06/108675), the FG072-03 event (see PCT Publication No. WO11/063411), the DAS-44406 event (see PCT Publication No. WO12/075426), the DAS-81419 event (see PCT Publication No. WO13/016527), the COR-23134 event, the GM_CSM63714 event (see PCT Publication No. WO23/212564), the GM_CSM63717 event (see PCT Publication No. WO2025/128414), the GM_CSM63770 event (see PCT Publication No. WO24/006698), the GMB151 event (see PCT Publication Nos. WO18/119364 and WO19/227036), the SHZD32 event (see PCT Publication No. WO17/059795), the DBN8002 event (see PCT Publication No. WO21/026688), the DBN9004-6 event (see PCT Publication No. WO17/215328), and the Verdeca HB4 (IND-00410) event (see PCT Publication No. WO20/197558).

In some embodiments, the second pest control agent may be derived from sources other than B. thuringiensis. For example, the second pest control agent can be an alpha-amylase, a peroxidase, a cholesterol oxidase, a patatin, a protease, a protease inhibitor, a urease, an alpha-amylase inhibitor, a pore-forming protein, a chitinase, a lectin, an engineered antibody or antibody fragment, a Bacillus cereus insecticidal protein, a Xenorhabdus spp. (such as X. nematophila or X. bovienii) insecticidal protein, a Photorhabdus spp. (such as P. luminescens or P. asymobiotica) insecticidal protein, a Brevibacillus spp. (such as B. laterosporous) insecticidal protein, a Lysinibacillus spp. (such as L. sphearicus) insecticidal protein, a Chromobacterium spp. (such as C. subtsugae or C. piscinae) insecticidal protein, a Yersinia spp. (such as Y. entomophaga) insecticidal protein, a Paenibacillus spp. (such as P. propylaea) insecticidal protein, a Clostridium spp. (such as C. bifermentans) insecticidal protein, a Pseudomonas spp. (such as P. fluorescens) and a lignin. In other embodiments, the second agent may be at least one insecticidal protein derived from an insecticidal toxin complex (Tc) from Photorhabdus, Xenorhabus, Serratia, or Yersinia. In other embodiments, the insecticidal protein may be an ADP-ribosyltransferase derived from an insecticidal bacteria, such as Photorhabdus ssp. In other embodiments, the insecticidal protein may be a VIP protein, such as VIP1 and/or VIP2 from B. cereus. In still other embodiments, the insecticidal protein may be a binary toxin derived from an insecticidal bacteria, such as ISP1A and ISP2A from B. laterosporous or BinA and BinB from L. sphaericus. In still other embodiments, the insecticidal protein may be engineered or may be a hybrid or chimera of any of the preceding insecticidal proteins.

Other example second pest controls agents include DIG-657 (US Patent Publication 2015366211); PtIP-96 (US Patent Publication 2017233440); PIP-72 (US Patent Publication US2016366891); PIP-83 (US Patent Publication 2016347799); PIP-50 (US Patent Publication 2017166921); IPD73 (US Patent Publication 2019119334); IPD090 (US Patent Publication 2019136258); IPD80 (US Patent Publication 2019256563); IPD078, IPD084, IPD086, IPD087, IPD089 (US Patent Publication 2020055906); IPD093 (International Application Publication WO2018111551); IPD059 (International Application Publication WO2018232072); IPD113 (International Application Publication WO2019178042); IPD121 (International Application Publication WO2018208882); IPD110 (International Application Publication WO2019178038); IPD103 (International Application Publication WO2019125717); IPD092; IPD095; IPD097; IPD099; IPD100, IPD105; IPD106; IPD107; IPD111; IPD112 (International Application Publication WO2020055885); IPD102 (International Application Publication WO2020076958) Cry1B.868 and Cry1 Da_7 (US Patent Publication 2020-032289); TIC107 (U.S. Pat. No. 8,049,071); Cry2Ab and Cry1A.105 (U.S. patent Ser. No. 10/584,391); Cry1F, Cry34Ab1, Cry35Ab1 (U.S. patent Ser. No. 10/407,688); TIC6757, TIC7472, TIC7473, TIC6757 (US Patent Publication 2017058294); TIC3668, TIC3669, TIC3670, TIC4076, TIC4078, TIC4260, TIC4346, TIC4826, TIC4861, TIC4862, TIC4863, TIC-3668 (US Patent Publication 2016319302); TIC7040, TIC7042, TIC7381, TIC7382, TIC7383, TIC7386, TIC7388, TIC7389 (US Patent Publication 2018291395); TIC7941 (US Patent Publication 2020229445) TIC836, TIC860, TIC867, TIC868, TIC869, and TIC1100 (International Application Publication WO2016061391), TIC2160 (International Application Publication WO2016061392), ET66, TIC400, TIC800, TIC834, TIC1415, AXMI-001, AXMI-002, AXMI-030, AXMI-035, AND AXMI-045 (US Patent Publication 20130117884), AXMI-52, AXMI-58, AXMI-88, AXMI-97, AXMI-102, AXMI-112, AXMI-117, AXMI-100 (US Patent Publication 201-0310543), AXMI-115, AXMI-113, AXMI-005 (US Patent Publication 20130104259), AXMI-134 (US Patent Publication 20130167264), AXMI-150 (US Patent Publication 20100160231), AXMI-184 (US Patent Publication 20100004176), AXMI-196, AXMI-204, AXMI-207, AXMI-209 (US Patent Publication 2011-0030096), AXMI-218, AXMI-220 (US Patent Publication 20140245491), AXMI-221z, AXMI-222z, AXMI-223z, AXMI-224z, AXMI-225z (US Patent Publication 20140196175), AXMI-238 (US Patent Publication 20140033363), AXMI-270 (US Patent Publication 20140223598), AXMI-345 (US Patent Publication 20140373195), AXMI-335 (International Application Publication WO2013134523), DIG-3 (US Patent Publication 20130219570), DIG-5 (US Patent Publication 20100317569), DIG-11 (US Patent Publication 20100319093), AfIP-1A (US Patent Publication 20140033361), AfIP-1B (US Patent Publication 20140033361), PIP-1APIP-1B (US Patent Publication 20140007292), PSEEN3174 (US Patent Publication 20140007292), AECFG-592740 (US Patent Publication 20140007292), Pput_1063 (US Patent Publication 20140007292), DIG-657 (International Application Publication WO2015195594), Pput_1064 (US Patent Publication 20140007292), GS-135 (US Patent Publication 20120233726), GS153 (US Patent Publication 20120192310), GS154 (US Patent Publication 20120192310), GS155 (US Patent Publication 20120192310), DIG-911 and DIG-180 (US Patent Publication No. 20150264940); and the like.

In some embodiments, the second pesticidal agent can be non-proteinaceous, for example, an interfering RNA molecule such as a dsRNA, which can be expressed transgenically or applied as part of a composition (e.g., using topical methods). An interfering RNA typically comprises at least a RNA fragment against a target gene, a spacer sequence, and a second RNA fragment which is complementary to the first, so that a double-stranded RNA structure can be formed. RNA interference (RNAi) occurs when an organism recognizes double-stranded RNA (dsRNA) molecules and hydrolyzes them. The resulting hydrolysis products are small RNA fragments of about 19-24 nucleotides in length, called small interfering RNAs (siRNAs). The siRNAs then diffuse or are carried throughout the organism, including across cellular membranes, where they hybridize to mRNAs (or other RNAs) and cause hydrolysis of the RNA. Interfering RNAs are recognized by the RNA interference silencing complex (RISC) into which an effector strand (or “guide strand”) of the RNA is loaded. This guide strand acts as a template for the recognition and destruction of the duplex sequences. This process is repeated each time the siRNA hybridizes to its complementary-RNA target, effectively preventing those mRNAs from being translated, and thus “silencing” the expression of specific genes from which the mRNAs were transcribed. Interfering RNAs are known in the art to be useful for insect control (see, for example, publication WO2013/192256, incorporated by reference herein). An interfering RNA designed for use in insect control produces a non-naturally occurring double-stranded RNA, which takes advantage of the native RNAi pathways in the insect to trigger down-regulation of target genes that may lead to the cessation of feeding and/or growth and may result in the death of the insect pest. The interfering RNA molecule may confer insect resistance against the same target pest as the disclosed proteins or may target a different pest. The targeted insect plant pest may feed by chewing, sucking, or piercing. Interfering RNAs are known in the art to be useful for insect control. In some embodiments, the dsRNA useful for insect control is described in US Patent Publications 20190185526, 2018020028 or 20190177736. In some embodiments, the dsRNA useful for insect control is described in U.S. Pat. No. 9,238,8223, 9,340, 797, or 8,946,510. In some embodiments, the dsRNA useful for insect control is described in U.S. Patent Publications 20200172922, 20110054007, 20140275208, 20160230185, or 20160230186. In other embodiments, the interfering RNA may confer resistance against a non-insect plant pest, such as a nematode pest or a virus pest.

In still further embodiments, the first insect control agent, which is a disclosed insecticidal protein and the second pest control agent are co-expressed in a transgenic plant. This co-expression of more than one pesticidal principle in the same transgenic plant can be achieved by genetically engineering a plant to contain and express the nucleic acid sequences encoding the insect control agents. For example, the co-expression of more than one pesticidal agent in the same transgenic plant can be achieved by making a single recombinant vector comprising coding sequences of more than one pesticidal agent in a “molecular stack” and genetically engineering a plant to contain and express all the pesticidal agents in the transgenic plant. Such molecular stacks may also be made by using mini-chromosomes as described, for example in U.S. Pat. No. 7,235,716. Alternatively, a plant, Parent 1, can be genetically engineered for the expression of the disclosed insecticidal proteins. A second plant, Parent 2, can be genetically engineered for the expression of a second pest control agent. By crossing Parent 1 with Parent 2, progeny plants are obtained which express both insect control agents from Parents 1 and 2.

In other embodiments, the disclosure provides a stacked transgenic plant resistant to plant pest infestation comprising a nucleic acid (e.g., DNA) sequence encoding a dsRNA for suppression of an essential gene in a target pest and a nucleic acid e.g., (DNA) sequence encoding a disclosed insecticidal protein exhibiting insecticidal activity against the target pest.

Transgenic plants or seed comprising and/or expressing a disclosed protein can also be treated with an insecticide or insecticidal seed coating as described in U.S. Pat. Nos. 5,849,320 and 5,876,739. In some embodiments, where both the insecticide or insecticidal seed coating and the transgenic plant or seed of the disclosure are active against the same target insect, for example a coleopteran pest (e.g., Western corn rootworm), the combination is useful (i) in a method for further enhancing activity of the composition of the disclosure against the target insect, and/or (ii) in a method for preventing development of resistance to the composition of the disclosure by providing yet another mechanism of action against the target insect. Thus, in some embodiments, a method is provided of enhancing control of a coleopteran insect population comprising providing a transgenic plant or seed of the disclosure and applying to the plant or the seed an insecticide or insecticidal seed coating to a transgenic plant or seed of the disclosure.

Even where the insecticide or insecticidal seed coating is active against a different insect, the insecticide or insecticidal seed coating is useful to expand the range of insect control, for example by adding an insecticide or insecticidal seed coating that has activity against coleopteran insects to a transgenic seed of the disclosure, which, in some embodiments, has activity against lepidopteran insects, the coated transgenic seed produced controls both lepidopteran and coleopteran insect pests.

### Methods of Making and Using the Insecticidal Proteins, Nucleic Acids, and Transgenic Plants

In addition to providing compositions, the disclosure also provides methods of producing and using an insecticidal protein of the disclosure. In some embodiments, the method of producing comprises culturing a transgenic non-human host cell that comprises a polynucleotide, expression cassette or vector that expresses a described insecticidal protein under conditions in which the host cell produces the insecticidal protein that is toxic to the coleopteran pest. In some embodiments, the transgenic non-human host cell is a plant cell. In some other embodiments, the plant cell is a maize cell. In other embodiments, the conditions under which the plant cell are grown include natural sunlight. In other embodiments, the transgenic non-human host cell is a bacterial cell. In still other embodiments, the transgenic non-human host cell is a yeast cell.

In some embodiments, the methods of the disclosure provide control of at least one coleopteran pest, including without limitation, one or more of the following: Diabrotica barberi (northern corn rootworm), D. virgifera virgifera (western corn rootworm), D. undecimpunctata howardii (southern corn rootworm), D. balteata (banded cucumber beetle), D. undecimpunctata undecimpunctata (western spotted cucumber beetle), D. significata (3-spotted leaf beetle), D. speciosa (chrysanthemum beetle), D. virgifera zeae (Mexican corn rootworm), D. beniensis, D. cristata, D. curviplustalata, D. dissimilis, D. elegantula, D. emorsitans, D. graminea, D. hispanloe, D. lemniscata, D. linsleyi, D. milleri, D. nummularis, D. occlusal, D. porrecea, D. scutellata, D. tibialis, D. trifasciata and D. viridula; and any combination thereof. Other nonlimiting examples of Coleopteran insect pests include Leptinotarsa spp. such as L. decemlineata (Colorado potato beetle); Chrysomela spp. such as C. scripta (cottonwood leaf beetle); Hypothenemus spp. such as H. hampei (coffee berry borer); Sitophilus spp. such as S. zeamais (maize weevil); Epitrix spp. such as E. hirtipennis (tobacco flea beetle) and E. cucumeris (potato flea beetle); Phyllotreta spp. such as P. cruciferae (crucifer flea beetle) and P. pusilla (western black flea beetle); Anthonomus spp. such as A. eugenii (pepper weevil); Hemicrepidus spp. such as H. memnonius (wireworms); Melanotus spp. such as M. communis (wireworm); Ceutorhychus spp. such as C. assimilis (cabbage seedpod weevil); Phyllotreta spp. such as P. cruciferae (crucifer flea beetle); Aeolus spp. such as A. mellillus (wireworm); Aeolus spp. such as A. mancus (wheat wireworm); Horistonotus spp. such as H. uhlerii (sand wireworm); Sphenophorus spp. such as S. maidis (maize billbug), S. zeae (timothy billbug), S. parvulus (bluegrass billbug), and S. callosus (southern corn billbug); Phyllophaga spp. (White grubs); Chaetocnema spp. such as C. pulicaria (corn flea beetle); Popillia spp. such as P. japonica (Japanese beetle); Epilachna spp. such as E. varivestis (Mexican bean beetle); Cerotoma spp. such as C. trifurcate (Bean leaf beetle); Epicauta spp. such as E. pestifera and E. lemniscata (Blister beetles), Holotrichia spp. such as H. diomphalia Bates (Northeast larger black chafer); and any combination of the foregoing. In some embodiments, the insecticidal protein has insecticidal activity against a Western corn rootworm colony that is resistant to one or more insecticidal proteins present in a commercial product, such as mCry3a, eCry3.1Ab, Cry3Bb1, Cry34/35, IPD072Aa, IPD079Ea, Cry75Aa1, or Vip4Da2.

Also encompassed are methods of producing an insect-resistant (e.g., a coleopteran insect-resistant) transgenic plant. In some embodiments, the method comprises: introducing into a plant a polynucleotide, expression cassette or vector comprising a nucleotide sequence that encodes a disclosed insecticidal protein, wherein the nucleotide sequence is expressed in the plant to produce the disclosed insecticidal protein, thereby conferring to the plant resistance to the insect pest, and producing an insect-resistant transgenic plant (e.g., as compared with a suitable control plant, such as a plant that does not comprise the disclosed polynucleotide, expression cassette or vector and/or does not express a disclosed insecticidal polypeptide).

In some embodiments, a pest-resistant transgenic plant is resistant to an insect pest selected from the group consisting of Diabrotica virgifera virgifera (western corn rootworm; WCR), Diabrotica barberi (northern corn rootworm; NCR), and/or Diabrotica undecimpunctata howardi (southern corn rootworm; SCR) and/or other Diabrotica species including Diabrotica virgifera zeae (Mexican corn rootworm).

In some embodiments, the method of introducing the disclosed polynucleotide, expression cassette or vector into the plant comprises first transforming a plant cell with the polynucleotide, expression cassette or vector and regenerating a transgenic plant therefrom, where the transgenic plant comprises the polynucleotide, expression cassette or vector and expresses the disclosed chimeric insecticidal protein of the disclosure.

Alternatively, or additionally, the introducing step can comprise crossing a first plant comprising the polynucleotide, expression cassette or vector with a second plant (e.g., a different plant from the first plant, for example, a plant that does not comprise the polynucleotide, expression cassette or vector) and, optionally, producing a progeny plant that comprises the polynucleotide, expression cassette or vector and expresses a disclosed insecticidal protein, thereby resulting in increased resistance to at least one insect pest. Thus, a transgenic plant encompasses a plant that is the direct result of a transformation event and the progeny thereof (of any generation) that comprise the polynucleotide, expression cassette or vector and optionally expresses the insecticidal protein resulting in increased resistance to at least one insect pest. Once a desired nucleic acid molecule has been transformed into a particular plant species, it may be propagated in that species or moved into other varieties of the same species, particularly including commercial varieties, using traditional breeding techniques.

The disclosure further provides a method of identifying a transgenic plant of the disclosure, the method comprising detecting the presence of a polynucleotide, expression cassette, vector or insecticidal protein of the disclosure in a plant (or a plant cell, plant part, and the like derived therefrom), and thereby identifying the plant as a transgenic plant of the disclosure based on the presence of the polynucleotide, expression cassette, vector or insecticidal protein of the disclosure.

Embodiments further provide a method of producing a transgenic plant with increased resistance to at least one insect pest (e.g., a least one lepidopteran pest), the method comprising: planting a seed comprising a polynucleotide, expression cassette or vector of the disclosure, and growing a transgenic plant from the seed, where the transgenic plant comprises the polynucleotide, expression cassette or vector and produces the insecticidal protein.

In some embodiments, transgenic plants produced by the methods of the disclosure comprise a polynucleotide, expression cassette or vector of the disclosure. In some embodiments, a transgenic plant produced by the methods of the disclosure comprise an insecticidal protein of the disclosure and, optionally have increased resistance to at least one insect pest.

The methods of producing a transgenic plant described herein optionally comprise a further step of harvesting a seed from the transgenic plant, where the seed comprises the polynucleotide, expression cassette or vector and produces the insecticidal protein. Optionally, the seed produces a further transgenic plant that comprises the polynucleotide, expression cassette or vector and produces the insecticidal protein, and thereby has increased resistance to at least one insect pest.

The disclosure further provides plant parts, plant cells, plant organs, plant cultures, seed, plant extracts, harvested products and processed products of the transgenic plants produced by the methods of the disclosure.

As a further aspect, the disclosure also provides a method of producing seed, the method comprising: providing a transgenic plant that comprises a disclosed polynucleotide, expression cassette or vector, and harvesting a seed from the transgenic plant, wherein the seed comprises the polynucleotide, expression cassette, vector and produces the insecticidal protein. Optionally, the seed produces a further transgenic plant that comprises the polynucleotide, expression cassette or vector and produces the insecticidal protein, and thereby has increased resistance to at least one insect pest. In representative embodiments, the step of providing the transgenic plant comprises planting a seed that produces the transgenic plant.

Further provided is a method of producing a hybrid plant seed, the method comprising: crossing a first inbred plant, which is a transgenic plant comprising a polynucleotide, expression cassette or vector of the disclosure, and optionally expressing an insecticidal protein of the disclosure with a different inbred plant (e.g., an inbred plant that does not comprise a polynucleotide, expression cassette or vector of the disclosure) and allowing hybrid seed to form. Optionally, the method further comprises harvesting a hybrid seed. In some embodiments, the hybrid seed comprises the polynucleotide, expression cassette or vector of the disclosure, and in some embodiments may further comprise an insecticidal protein of the disclosure and have increased resistance to an insect pest. In some embodiments, the hybrid seed produces a transgenic plant that comprises the polynucleotide, expression cassette or vector of the disclosure, expresses the insecticidal protein of the disclosure, and has increased resistance to at least one insect pest.

In further embodiments, a method of controlling a coleopteran pest is provided, the method comprising delivering to the pests or an environment containing such pests an effective amount of a disclosed insecticidal protein. To be effective, the insecticidal protein is first orally ingested by the insect. However, the insecticidal protein can be delivered to the insect in many ways. The ways to deliver a protein orally to an insect include, but are not limited to, providing the protein (1) in a transgenic plant, wherein the insect eats (ingests) one or more parts of the transgenic plant, thereby ingesting the polypeptide that is expressed in the transgenic plant; (2) in a formulated protein composition(s) that can be applied to or incorporated into, for example, insect growth media; (3) in a protein composition(s) that can be applied to the surface, for example, sprayed, onto the surface of a plant part, which is then ingested by the insect as the insect eats one or more of the sprayed plant parts; (4) a bait matrix; or (5) any other art-recognized protein delivery system. Thus, any method of oral delivery to an insect can be used to deliver the disclosed insecticidal proteins of the disclosure. In some particular embodiments, the disclosed insecticidal protein is delivered orally to an insect, wherein the insect ingests one or more parts of a transgenic plant.

In other embodiments, the disclosed insecticidal protein is delivered orally to an insect, wherein the insect ingests one or more parts of a plant covered or partially covered with a composition comprising the insecticidal proteins. Delivering the compositions of the disclosure to a plant surface can be done using any method known to those of skill in the art for applying compounds, compositions, formulations and the like to plant surfaces. Some non-limiting examples of delivering to or contacting a plant or part thereof include spraying, dusting, sprinkling, scattering, misting, atomizing, broadcasting, soaking, soil injection, soil incorporation, drenching (e.g., root, soil treatment), dipping, pouring, coating, leaf or stem infiltration, side dressing or seed treatment, and the like, and combinations thereof. These and other procedures for contacting a plant or part thereof with compound(s), composition(s) or formulation(s) are well-known to those of skill in the art.

In some embodiments, the disclosed nucleotide and/or polypeptide sequences can be used in a bioinformatic analysis to identify additional insecticidal toxins, e.g., one or both of the nucleotide sequences and the proteins encoded by the nucleic acids. In some embodiments, this identification of additional toxins can be based on percent identity (e.g., using a BLAST, Needle, or similar algorithm). In other embodiments, the identification of additional toxins could be accomplished using conserved protein domains or epitopes (e.g., using Pfam, Hmmer, psi-BLAST, or hhsuite). In some embodiments, the bioinformatic assay comprises running a sequence identity comparison and selecting one or more candidate insecticidal toxins that has a sequence identity above a certain threshold (e.g., at least 30%, at least 40%, at least 50%, at least 60%, at least 70%, at least 80%, at least 90%, or more identical) relative to a disclosed nucleotide or polypeptide sequence of the disclosure. In some embodiments, the bioinformatic assay comprises running a domain or epitope conservation analysis and selecting one or more candidate insecticidal toxins that has at least one conserved domain or epitope relative to a disclosed nucleotide or polypeptide sequence of the disclosure.

In some embodiments, the bioinformatic identification of the additional toxins could be accomplished using computational methods (e.g., machine learning models and optimization algorithms) to select candidate insecticidal toxins. For example, such a method may involve an iterative process of generating candidate toxin sequences, evaluating their structural, sequence, and physical properties using machine learning models, deep learning models, statistical physics-based models, and/or sequence analysis protocols, and selectively modifying the sequences to optimize toward desired characteristics such as insecticidal properties.

In some embodiments, a starting sequence of a polypeptide of the disclosure is provided as input to a generative model, a statistical physics-based model, or other model. The models produce a set of candidate protein toxin sequences that diverge from the starting sequence while maintaining key structural motifs and/or functional regions. These candidate toxin sequences can be generated by techniques such as (a) mutating residues at selected positions in the starting sequence, guided by evolutionary conservation data, structural constraints, and physics and knowledge-based potentials, (b) recombining fragments from diverse protein families to graft new structural elements onto the starting sequence backbone, or (c) using generative adversarial networks (GANs), protein language models (pLMs), or other deep learning models to generate entirely novel sequences optimized for desired properties such as insecticidal activity. The resulting candidate toxin sequences may then be evaluated by a series of models, such as AlphaFold2, RoseTTAFold, Rosetta, or language models, to evaluate desired characteristics such as insecticidal activity. This process may involve the identification of key domains or regions within the protein structure that are important for the desired function or property and the evaluation of these regions' properties in the candidate sequences. Such identification can be done using activity data (e.g., insecticidal activity based on bioassays) and/or using conserved domain identification through sequence analysis with homologues, such as orthologs. Candidate toxin sequence selection may be accomplished through evaluation and scoring of the generated candidate protein sequences based on various criteria, including structural quality, energy minimization, and predicted functional properties. Examples of such methods are described, e.g., in patent application publications CN116768990 and WO2024095126, as well as in Sumida et al. Improving Protein Expression, Stability, and Function with ProteinMPNN. Journal of the American Chemical Society. 2024, 146(3): 2054-2061 and Murphy et al. Increasing Sequence Diversity with Flexible Backbone Protein Design: The Complete Redesign of a Protein Hydrophobic Core. Structure. 2012, 20(6): 1086-1096. In some embodiments, a method of identifying a candidate insecticidal toxin using computational methods comprises: a) providing a three-dimensional structure of a polypeptide of the disclosure (e.g.; computationally such as with Alphafold2 or by crystallography); b) identifying active regions or positions within the polypeptide amino acid sequence that contribute to insecticidal activity (e.g., using the bioassay activity data such as the data disclosed herein and/or conserved regions or positions through sequence analysis with homologues, such as orthologs); c) computationally generating a plurality of putative toxin amino acid sequences, wherein the active regions or positions are maintained (e.g., at at least 90%, at least 95%, at least 95%, at least 95%, at least 95%, at least 95%, at least 95%, at least 95%, at least 95%, at least 95%, or 100% identity relative to the regions and/or positions in the polypeptide amino acid sequence) and at least one other portion of the polypeptide amino acid sequence is modified (e.g., through one or more amino acid substitution, deletion, or addition) to include at least one difference relative to the unmodified polypeptide amino acid sequence (e.g., modified so that it is less than 95%, less than 90%, less than 80%, less than 70%, less than 60%, less than 50%, less than 40%, less than 30%, less than 20% or less than 10%, or between 10% to 90%, 20% to 90%, 30% to 90%, 40% to 90%, 50% to 90%, 60% to 90%, 70% to 90%, 80% to 90%, 10% to 80%, 20% to 80%, 30% to 80%, 40% to 80%, 50% to 80%, 60% to 80%, 70% to 80%, 10% to 70%, 20% to 70%, 30% to 70%, 40% to 70%, 50% to 70%, or 60% to 70% identical to the corresponding unmodified portion of the polypeptide amino acid sequence); d) predicting three-dimensional structures for the plurality of putative toxin sequences; e) evaluating whether the three-dimensional structures of the plurality of putative toxin sequences are similar to the three-dimensional structure of the polypeptide amino acid sequence (e.g., using criteria such as structural quality, energy minimization, and predicted insecticidal properties); and f) selecting candidate insecticidal toxins based on the evaluation in step e). In some embodiments, the method comprises repeating step c) based on the results of step e) to develop further candidate insecticidal toxins.

In some embodiments, any of the above-mentioned methods may further comprise determining the insecticidal activity of one or more candidate insecticidal toxins identified using the bioinformatic analysis.

In some embodiments, determination of insecticidal activity of disclosed insecticidal proteins or candidate insecticidal toxins can be accomplished through an insect bioassay. Insect bioassay methods are well known in the art and can be “in vitro” or “in planta”. In in vitro bioassays, the disclosed proteins or candidate insecticidal toxins are delivered to the desired insect species following production in a cell-based system, e.g., recombinant bacterial strains (e.g., E. coli, Bacillus thurinigiensis Cry-). Clarified lysates containing the disclosed proteins or candidate insecticidal toxins produced in these recombinant bacterial strains can be fed orally to the insects. Alternatively, purified proteins can be prepared and fed orally to the insects. In some embodiments, the clarified lysate or purified protein is overlaid on artificial diet prior to infestation with the insects. In other embodiments, the clarified lysate or purified protein is mixed into or incorporated into the artificial diet prior infestation with insects. In in planta bioassays, transgenic plants expressing the disclosed proteins or candidate insecticidal toxins are utilized to deliver the toxin to the desired insect species. In some embodiments, sampled tissue is fed orally to the insects. Nonlimiting examples of sampled tissue include leaf, root, pollen, silk, and stem. In some embodiments the plant tissue is mixed into or incorporated into artificial diet prior to infestation with the insects. In some embodiments, the evaluated insects are LI instars or neonates. In other embodiments, the evaluated insects are of later larval stages, namely L2, L3, L4, or L5 instars.

### EXAMPLES

Embodiments of the invention can be better understood by reference to the following detailed examples. The foregoing and following description of embodiments of the invention and the various embodiments are not intended to limit the claims but are rather illustrative thereof. Therefore, it will be understood that the claims are not limited to the specific details of these examples. It will be appreciated by those skilled in the art that other embodiments of the invention may be practiced without departing from the spirit and the scope of the disclosure, the scope of which is defined by the appended claims. Art recognized recombinant DNA and molecular cloning techniques may be found in, for example, J. Sambrook, et al., Molecular Cloning: A Laboratory Manual, 4th Ed., Cold Spring Harbor, NY: Cold Spring Harbor Laboratory Press (2012); by T. J. Silhavy, M. L. Berman, and L. W. Enquist, Experiments with Gene Fusions, Cold Spring Harbor Laboratory, Cold Spring Harbor, NY (1984) and by Ausubel, F. M. et al., Current Protocols in Molecular Biology, New York, John Wiley and Sons Inc., (1988), Reiter, et al., Methods in Arabidopsis Research, World Scientific Press (1992), and Schultz et al., Plant Molecular Biology Manual, Kluwer Academic Publishers (1998).

### Example 1: Identification of Proteins with Insecticidal Activity Against Western Corn Rootworm

An insecticidal protein, Mv6_CRW was identified from the fern species Marsilea vestita. E. coli-optimized versions of the genes were synthesized, and the genes cloned into a pET29a vector. The resulting constructs were transformed into E. coli BL21*(DE3) and protein expression carried out in Luria-Bertani broth with IPTG inductions at 18 °C overnight. Cultures were harvested the next morning, and pellets were resuspended in 50 mM Tris pH 8.5, 50 mM NaCl. Buffer. The soluble fraction of lysates was prepared from these cultures by use of a French pressure cell followed by centrifugation of whole lysates at 20,000×g for thirty minutes. Lysates were de-salted using Zeba spin columns and converted to 50 mM potassium phosphate, pH 7.0, 50 mM NaCl by buffer exchange. The lysate preparation was then tested for bioactivity to Western Corn Rootworm (WCR; Diabrotica virgifera).

Bioactivity assays were performed using a diet-incorporation method. Briefly, E. coli BL21*(DE3) lysates were mixed with an equal volume of heated artificial insect diet (Bioserv, Inc., Frenchtown, NJ) in 1.5 mL centrifuge tubes and then applied to small petri-dishes. After the diet-sample mixture cooled and solidified, 12 WCR larvae were added to each plate. The plates were sealed and maintained at ambient laboratory conditions with regard to temperature, lighting, and relative humidity. Lysates from E. coli BL21* (DE3) cultures harboring the empty pET29a vector were used as negative controls. Mortality was assessed on day 3 or 4 and again on day 6 or 7. For this and all subsequent tables, a “-” means no mortality, a “+” means 1-24% mortality, a “++” means 25-49% mortality, a “+++” means 50-74% mortality, and a “++++” means 75-100% mortality. For this and all subsequent tables showing insecticidal activity on CRW, the abbreviations for the “Remarks” column are as follows: s=small larvae, sm=small/medium larvae, m=medium larvae, mb=medium/big larvae, b=big larvae, vb=very big larvae. For this and all subsequent tables showing the insecticidal activity of identified proteins or variants thereof, the “SEQ ID NO.” refers to the amino acid sequence of the protein.

As shown in Table 3, lysates from cultures expressing the identified protein Mv6_CRW displayed strong bioactivity against WCR. Mv6_CRW was also tested against a number of Lepidopterans including North American fall armyworm, and soybean looper. There was little to no insecticidal effect on these other pests.

As shown in Tables 4 through 6, lysates from the cultures expressing the identified orthologue proteins likewise displayed bioactivity against WCR. Replicate bioassays using additional lysates expressing these proteins were performed and yielded similar results.

TABLE 3
Insecticidal activity of Mv6_CRW_v1 lysates against Western Corn Rootworm (WCR)

| | | Day 4 | | | Day 6 | | |
|---|---|---|---|---|---|---|---|
| Treatment | SEQ ID NO: | n | Mortality | Remarks | n | Mortality | Remarks |
| BL21*/pET29a-empty | | 12 | − | b | 12 | + | b |
| BL21*/Mv6_CRW_v1 | 1 | 12 | +++ | s | 12 | ++++ | |

TABLE 4
Insecticidal activity of Mv6_CRW_v2 lysates against Western Corn Rootworm (WCR)

| | | Day 4 | | | Day 6 | | |
|---|---|---|---|---|---|---|---|
| Treatment | SEQ ID NO: | n | Mortality | Remarks | n | Mortality | Remarks |
| BL21*/pET29a-empty | | 12 | − | b | 12 | − | b |
| BL21*/Mv6_CRW_v2 | 2 | 12 | +++ | s | 12 | ++++ | |

TABLE 5
Insecticidal activity of Mv6_CRW_v3 lysates against Western Corn Rootworm (WCR)

| | | Day 4 | | | Day 6 | | |
|---|---|---|---|---|---|---|---|
| Treatment | SEQ ID NO: | n | Mortality | Remarks | n | Mortality | Remarks |
| BL21*/pET29a-empty | | 12 | − | b | 12 | + | b |
| BL21*/Mv6_CRW_v3 | 3 | 12 | + | mb | 12 | ++++ | m/mb |

TABLE 6
Insecticidal activity of Mv6_CRW_v4 lysates against Western Corn Rootworm (WCR)

| | | Day 4 | | | Day 6 | | |
|---|---|---|---|---|---|---|---|
| Treatment | SEQ ID NO: | n | Mortality | Remarks | n | Mortality | Remarks |
| BL21*/pET29a-empty | | 12 | − | b | 12 | − | b |
| BL21*/Mv6_CRW_v4 | 4 | 12 | ++++ | mb | 12 | ++++ | |

### Example 2: Purified Mv6_CRW Proteins are Insecticidal Against WCR

Mv6_CRW_v1 and v3 proteins (SEQ ID NOs:1 and 3, respectively) were purified to further characterize their insecticidal properties. An N-terminal 6 Histidine+5 residue linker (MHHHHHHGGGGS, SEQ ID NO: 17) pET29a construct (designated p6HISMv6) was generated for Mv6_CRW_v1 and v3 to facilitate protein purification. Two liters of E. coli BL21* (DE3) cells harboring p6HISMv6 were grown at 37 °C in LB media. IPTG (1 mM) was added to the cultures when the O.D. reached 0.8-1.0 and then the cultures were moved to 18 °C for 18 hours. The cell pellet was harvested and re-suspended in 20 mM Tris, pH 8.0, 0.5 M NaCl, 10% glycerol, and 5 mM Beta-mercaptoethanol (Buffer A). The cells were lysed using a French pressure cell; the lysate was then spun at 100k×g in an ultracentrifuge for 1 hour. The supernatant fractions for Mv6_CRW_v1 and v3 were collected and then syringe-filtered before loading onto a 5 mL HisTrapFF FPLC column that was pre-equilibrated in Buffer A. The HisTrapFF column bound Mv6_CRW_v1 and v3 effectively; the proteins were eluted from the column using a linear imidazole gradient. The elution buffer consisted of Buffer A with 250 mM imidazole.

The purest fractions collected for the proteins were pooled and then dialyzed into 50 mM Tris pH 8.0, 50 mM NaCl. After dialysis, the proteins were concentrated by ultrafiltration prior to storage at −80 °C. Protein concentration for Mv6_CRW_v1 was determined by the BCA protein assay. Protein concentration for Mv6_CRW_v3 was determined by gel densitometry (using a BSA standard curve) due to the low yield and purity of this variant. The purified proteins were then tested against 12 WCR larvae over a range of concentrations in the diet-incorporation method, essentially as described in Example 1. As shown in Tables 7 and 8, the proteins display concentration dependent bioactivity to WCR over the range of concentrations tested. Additional replicates of the protein samples were prepared and tested in the bioassay and yielded similar results.

TABLE 7
Dose Response for purified Mv6_CRW_v1 protein against WCR

| | | Day 4 | | Day 6 | | Day 7 | |
|---|---|---|---|---|---|---|---|
| Treatment | n | Mortality | Remarks | Mortality | Remarks | Mortality | Remarks |
| 50 mM Tris pH 8.0, 50 mM NaCl | 12 | − | b | − | b | − | b |
| Mv6_CRW_v1 500 μg/mL | 12 | ++++ | | ++++ | | ++++ | |
| Mv6_CRW_v1 300 μg/mL | 12 | +++ | 2sm | ++++ | | ++++ | |
| Mv6_CRW_v1 100 μg/mL | 12 | ++ | m/mb | ++++ | sm/m | ++++ | |
| Mv6_CRW_v1 50 μg/mL | 12 | + | m | +++ | m/mb | ++++ | m |
| Mv6_CRW_v1 30 μg/mL | 12 | + | m | +++ | m/mb | ++++ | mb |
| Mv6_CRW_v1 10 μg/ml | 12 | + | mb/b | ++ | mb/b | ++ | mb |
| Mv6_CRW_v1 3 μg/mL | 12 | − | mb/b | − | b | − | b |

TABLE 8
Dose Response for purified Mv6_CRW_v3 protein against WCR

| | | Day 4 | | Day 6 | |
|---|---|---|---|---|---|
| Treatment | n | Mortality | Remarks | Mortality | Remarks |

| | | | | |
|---|---|---|---|---|
| 50 mM Tris pH 8.0, 50 mM NaCl | 12 | − | − | b |
| Mv6_CRW_v3 40.6 μg/mL | 12 | +++ | ++++ | all dead |
| Mv6_CRW_v3 20.3 μg/mL | 12 | +++ | ++++ | m/mb |
| Mv6_CRW_v3 10.2 μg/mL | 12 | ++ | +++ | mb |
| Mv6_CRW_v3 5.1 μg/mL | 12 | + | ++ | mb/b |

### Example 3: Purified Mv6_CRW_v1 Protein Possesses Insecticidal Activity Against Northern Corn Rootworm

Mv6_CRW_v1 (SEQ ID NO: 1) was also tested for efficacy against Northern Corn Rootworm (NCR) larvae in a diet-incorporation assay, performed essentially as described in Example 2. The purified protein was tested against 12 NCR larvae over a range of concentrations in the diet-incorporation method essentially as described in Example 1. As shown in Table 9, the protein displays concentration dependent bioactivity to NCR over the range of concentrations tested. Additional replicates of the protein samples were prepared and tested in the bioassay and yielded similar results.

TABLE 9
Insecticidal activity of Mv6_CRW_v1 against Northern Corn Rootworm (NCR)

| | Day 5 | | | | Day 6 | |
|---|---|---|---|---|---|---|
| Treatment | n | Mortality | Remarks | n | Mortality | Remarks |
| 50 mM Tris pH 8.0, 50 mM NaCl | 12 | − | b | 12 | − | b |
| Mv6_CRW_v1 100 μg/mL | 12 | ++++ | | 12 | ++++ | |
| Mv6_CRW_v1 50 μg/mL | 12 | ++++ | | 12 | ++++ | |
| Mv6_CRW_v1 25 μg/mL | 12 | ++++ | sm | 12 | ++++ | |
| Mv6_CRW_v1 12.5 μg/mL | 12 | ++++ | sm/m | 12 | ++++ | sm/m |

### Example 4: Purified Mv6_CRW_v1 Protein Possesses Insecticidal Activity Against Cry-Resistant WCR

To determine if the insecticidal activity of Mv6_CRW_v1 (SEQ ID NO: 1) is through a mode-of-action different from Cry3-related proteins, Mv6_CRW_v1 was purified as described above and were tested for efficacy against a strain of WCR that is resistant to the eCry3.1Ab protein (eCry3.1Ab-R) as well as the founder population used to develop the resistant colony. A susceptible colony of WCR that is not resistant to Cry proteins was used as a control in the bioassay. Diet-incorporation assays were performed essentially as described above, and mortality and growth inhibition observations, where s=small larvae, m=medium larvae and l=large larvae, were taken on days 4 and/or 6 post-infestation. The negative control consisted of 50 mM Tris pH 8.0, 50 mM NaCl, the storage buffer for purified Mv6_CRW_v1. Additional replicates of the protein samples were prepared and tested in the bioassay and yielded similar results. As shown in Table 10+Mv6_CRW_v1 has insecticidal activity against the eCry3.1Ab-resistant WCR strain indicating that Mv6_v1 has a unique mode-of-action compared to the commercialized CRW control protein.

TABLE 10
Insecticidal activity of Mv6_CRW_v1 at against Cry-resistant WCR

| | Crop | | | | |
|---|---|---|---|---|---|
| | Characteristics | | | | |
| | WCRW | Day 4 | | Day 6 | |
| Treatment | n | Mortality | Remarks | Mortality | Remarks |
| 50 mM Tris pH 8.0, 50 mM NaCl | 12 | − | | − | b |
| Mv6_CRW_v1 100 μg/mL | 12 | ++ | | ++++ | all dead |
| Mv6_CRW_v1 75 μg/mL | 12 | ++ | | ++++ | all dead |
| Mv6_CRW_v1 50 μg/mL | 12 | + | | ++++ | m |
| Mv6_CRW_v1 30 μg/mL | 12 | + | | ++++ | mb |
| Mv6_CRW_v1 20 μg/mL | 12 | − | | +++ | m/mb |
| Mv6_CRW_v1 10 μg/mL | 12 | − | | ++ | mb |
| Mv6_CRW_v1 5 μg/mL | 12 | − | | + | mb/b |
| Mv6_CRW_v1 2.5 μg/mL | 12 | − | | − | mb/b |

| | Founder WCRW | Day 4 | | Day 6 | |
|---|---|---|---|---|---|
| Treatment | n | Mortality | Remarks | Mortality | Remarks |
| 50 mM Tris pH 8.0, 50 mM NaCl | 12 | − | | − | b |
| Mv6_CRW_v1 100 μg/mL | 12 | ++++ | | ++++ | all dead |
| Mv6_CRW_v1 75 μg/mL | 12 | ++++ | | ++++ | all dead |
| Mv6_CRW_v1 50 μg/mL | 12 | ++++ | | ++++ | all dead |
| Mv6_CRW_v1 30 μg/mL | 12 | ++++ | | ++++ | all dead |
| Mv6_CRW_v1 20 μg/mL | 12 | +++ | | ++++ | all dead |
| Mv6_CRW_v1 10 μg/mL | 12 | +++ | | ++++ | all dead |
| Mv6_CRW_v1 5 μg/mL | 12 | +++ | | ++++ | all dead |
| Mv6_CRW_v1 2.5 μg/mL | 12 | ++ | | ++++ | mb |
| | eCry3.1Ab- | | | | |
| | resistant WCRW | Day 2 | | Day 6 | |
| Treatment | n | Mortality | Remarks | Mortality | Remarks |
| 50 mM Tris pH 8.0, 50 mM NaCl | 12 | + | | + | b |
| Mv6_CRW_v1 100 μg/mL | 12 | ++++ | | ++++ | all dead |
| Mv6_CRW_v1 75 μg/mL | 12 | ++++ | | ++++ | all dead |
| Mv6_CRW_v1 50 μg/mL | 12 | +++ | | ++++ | all dead |
| Mv6_CRW_v1 30 μg/mL | 12 | +++ | | ++++ | all dead |
| Mv6_CRW_v1 20 μg/mL | 12 | ++ | | ++++ | all dead |
| Mv6_CRW_v1 10 μg/mL | 12 | ++ | | ++++ | 1m |
| Mv6_CRW_v1 5 μg/mL | 12 | ++ | | ++++ | mb |
| Mv6_CRW_v1 2.5 μg/mL | 12 | ++ | | +++ | mb |

### Example 5: Maize Transformation

Transformation of immature maize embryos is performed essentially as described in Negrotto et al. (Plant Cell Reports (2000)19: 798-803). Briefly, Agrobacterium strain LBA4404 (pSBI) comprising an expression vector expressing the disclosed insecticidal proteins in Example 1 or one or more of any one of SEQ ID NO: 1, 2-4, 26, 27, 28, 29 or 30-473 is grown on YEP (yeast extract (5 g/L), peptone (10 g/L), NaCl (5 g/L), 15 g/l agar, pH 6.8) solid medium for 2-4 days at 28 °C. Approximately 0.8×109 Agrobacterium cells are suspended in LS-inf media supplemented with 100 μM As. Bacteria are pre-induced in this medium for approximately 30-60 minutes.

Immature embryos from an inbred maize line are excised from 8-12 day old ears into liquid LS-inf+100 μM As. Embryos are rinsed once with fresh infection medium. Agrobacterium solution is then added, and embryos are vortexed for 30 seconds and allowed to settle with the bacteria for 5 minutes. The embryos are then transferred scutellum side up to LSAs medium and cultured in the dark for two to three days. Subsequently, between approximately 20 and 25 embryos per petri plate are transferred to LSDc medium supplemented with cefotaxime (250 mg/l) and silver nitrate (1.6 mg/l) and cultured in the dark at approximately 28 °C for 10 days.

Immature embryos, producing embryogenic callus are transferred to LSD1M0.5S medium. The cultures are selected on this medium for approximately 6 weeks with a subculture step at about 3 weeks. Surviving calli are transferred to Reg1 medium supplemented with mannose. Following culturing in the light (16 hour light/8 hour dark regiment), green tissues are then transferred to Reg2 medium without growth regulators and incubated for about 1-2 weeks. Plantlets are transferred to Magenta GA-7 boxes (Magenta Corp, Chicago Ill.) containing Reg3 medium and grown in the light. After about 2-3 weeks, plants are tested for the presence of the selectable marker gene and the disclosed insecticidal genes by PCR. Positive plants from the PCR assay are transferred to a greenhouse for further evaluation.

### Example 6: Maize Leaf Sheath Assay

Basal stalks were harvested from 26-30 day old maize plants at the point of leaf expansion. The base of stalk to just above the apical meristem was trimmed and outer green leaves sheath were unwrapped until the large section of pale-yellow leaf bases are removed intact and laid flat on cutting surface. Pale-yellow tissue was the source material for leaf disc collection and treatment.

Transformation of maize leaf sheath discs was performed essentially as described in Horsch and Klee (Proc. Natl. Acad. Sci. USA (1986) 83: 4428-4432). Briefly, an Agrobacterium strain (e.g. EHA101) comprising an expression vector expressing the disclosed insecticidal proteins in Example 1 was grown in a bacterial selection medium (LB Broth (Miller): Tryptone (10 g/L), Yeast Extract (5 g/L), NaCl (10 g/L). +Kan50/Spec50) for 1-2 days at 28 °C. Late log phase cultures were spun down in 14 ml round bottom or 50 ml conical centrifuge tubes at 4000 rpm for 20 minutes at ~20 °C and re-suspended in 10 mM MgS04+400 uM Acetosyringone, 0.0025% v/v Silwet L77, adjusting cell suspension density to OD600=1.0 (normalize multiple cultures closely to 1.0=OD600). Maize leaf sheath discs were submerged into about 10 mL of the prepared Agrobacterium suspension and infiltrated within a syringe barrel by 3-4 cycles of creating and releasing vacuum force. After removing plunger and sample disks from syringe barrel, the sample disks were transferred to a folded stack of fresh large Kim wipes. Blotted disks were transferred to empty wells of a labeled 48-well plate and sealed. Sealed plates were placed into a 24 °C/~low light (eg. <50 uM)/16/8 hr photoperiod growth chamber for 3 days for infection and transient expression. After about 3 days, leaf sheath discs were tested for the presence of the protein via LC-MS and insecticidal activity (leaf protection/damage) by CRW bioassay.

For MV6_CRW_v1 quantitation by LC-MS, leaf sheath discs were homogenized using Klecko homogenizer. LC-MS extraction buffer was added at 400 ul per sample and the plate was shaken in Klecko again to extract total protein. Extracts were clarified by centrifugation and transferred to PCR plates. Samples were reduced with DTT and alkylated with iodacetamide before digestion. Digestion was carried out using Trypsin/LysC mixture at 1.10 ratio of enzyme to protein at 47 C, for 1 h 20 min, at 350 rpm (shaking). MV6_CRW_v1 Stable Isotopically Labeled (SIL) peptide was spiked into samples at 100 fmol per sample. Peptides were purified using solid phase extraction columns and fully dried.

For CRW bioassay, leaf sheath discs were transferred to a labeled 48-well plate containing 100 uL 1.5% agar. Sixteen (16) discs were assayed per construct/control material, at one disc per well. Plate wells containing disc samples were infested with three (3) neonate Western Corn Rootworm (Diabrotica virgifera virgifera, Crop Characteristics) larvae and sealed (Greiner Viewseal). Three #000 pinholes were added to each sealed well immediately following plate sealing. Plate images were captured following infestation and every 24 hours post-infestation for a three-day period. Infested plates were incubated within a secondary container alongside water-moistened Kimwipes as a humidity source, with primary containment within a Percival chamber (24.5 °C with 14 h light/10 h dark photoperiod); plates were only removed during 3-day course of assay for imaging. Following final imaging at day 3, plate wells were visually assessed for estimated area damage to leaf sheath disc sample as well as mortality or other status assessment of larvae. Data for estimated percentage of damaged leaf sheath disc area and insect mortality/size were recorded.

### Example 7: Transient Expression in Maize Plants

The presence and efficacy of the disclosed proteins in maize plants were tested by maize leaf sheath assay as described in Example 6. Additionally, multiple variants of CTP (chloroplast transit peptide) fused Mv6 were generated and evaluated. As shown in Table 11, the presence of Mv6 protein was detected by LC-MS (ng/mg total soluble protein (TSP)) in corn leaf sheath tissue samples from each construct containing CTPs (xOTPSSUct-08, xOsCTP6-01, xAtCTP2-01 and xSbCTP15-01) as well as without CTP fusion. From CRW bioassay, both Mv6_CRW_v1 alone and Mv6_CRW_v1 fused to a CTP provided plant tissue protection as indicated by minimal damage against WCR compared to WT tissue (which is infiltrated with Agrobacterium lacking binary vector). The lack of damage observed indicated that expression of the disclosed proteins in maize leaf sheath tissue provided protection from Western corn rootworm in a plant tissue-based bioassay.

TABLE 11
Transient Expression with Mv6_CRW_V1 CTP fusions

| | | | Protein | % |
|---|---|---|---|---|
| | | SEQ | expression | average |
| | | ID NO: | (ng Mv6/ | damage |
| | CTP | of CTP | mg TSP) | (±SD) |
| Wild Type | | | | 76.3 (13.0) |
| (no infiltration) | | | | |
| Mv6_CRW_v1 | none | | 8,497.15 | 0 (0) |
| Mv6_CRW_v1 | xOTPSSUct-08 | 19 | 2,356.99 | 6.3 (7.9) |
| Mv6_CRW_v1 | xOsCTP6-01 | 21 | 1,835.59 | 18.8 (26.8) |
| Mv6_CRW_v1 | xAtCTP2-01 | 23 | 1,555.91 | 35.6 (20.9) |
| Mv6_CRW_v1 | xSbCTP15-01 | 25 | 1,760.03 | 33.1 (29.9) |

### Example 8: Purified Mv6_CRW_v1 Protein Possesses Insecticidal Activity Against Cry-Resistant WCR

Susceptibility of Cry34/35-R, and susceptible (Crop Characteristics) WCR larvae to Mv6_CRW_v1 (SEQ ID NO:1), as well as to Cry34/35Ab protein was evaluated in 7-day diet overlay toxicity assays as described in Pereira et al (J Econ Entomol. 2020:113:1955-1962). As shown in Table 12, Mv6_CRW_v1 has insecticidal activity against the Cry34/35-resistant WCR strain indicating that Mv6_v1 has a unique mode-of-action compared to the commercialized CRW control protein.

TABLE 12
Insecticidal activity of Mv6_CRW_v1
at against Cry-resistant WCR

| | Susceptible | |
|---|---|---|
| | WCRW | Day 7 |
| Treatment | n | Mortality |
| 50 mM Tris pH 8.0, 50 mM NaCl | 8 | − |
| Mv6_CRW_v1 0.25 μg/cm2 | 12 | + |
| Mv6_CRW_v1 0.74 μg/cm2 | 12 | ++ |
| Mv6_CRW_v1 2.22 μg/cm2 | 12 | +++ |
| Mv6_CRW_v1 6.66 μg/cm2 | 12 | ++++ |
| Mv6_CRW_v1 20 μg/cm2 | 12 | ++++ |
| Mv6_CRW_v1 60 μg/cm2 | 12 | ++++ |
| Mv6_CRW_v1 200 μg/cm2 | 12 | ++++ |

| | Cry34/Cry35-R | |
|---|---|---|
| | WCRW | Day 7 |
| Treatment | n | Mortality |
| 50 mM Tris pH 8.0, 50 mM NaCl | 12 | + |
| Mv6_CRW_v1 0.25 μg/cm2 | 12 | + |
| Mv6_CRW_v1 0.74 μg/cm2 | 12 | +++ |
| Mv6_CRW_v1 2.22 μg/cm2 | 12 | ++++ |
| Mv6_CRW_v1 6.66 μg/cm2 | 12 | ++++ |
| Mv6_CRW_v1 20 μg/cm2 | 12 | ++++ |
| Mv6_CRW_v1 60 μg/cm2 | 12 | ++++ |
| Mv6_CRW_v1 200 μg/cm2 | 12 | ++++ |

### Example 9: Mv6_CRW_v1 Tested Against Lepidopteran Panel

Lysates from bacterial cultures expressing Mv6_CRW_v1 (SEQ ID NO: 1) were tested for bioactivity on a panel of Lepidopteran insects using a surface-overlay method. The panel of Lepidopteran insects included fall armyworm, corn earworm, black cutworm, soybean looper, European corn borer, southwestern corn borer, and velvet bean caterpillar. Briefly, 30 μL of E. coli BL21*(DE3) lysate harboring a vector with the Mv6_CRW_v1 gene was applied to 24 wells of diet in a 48 well plate. Lysates from E. coli BL21* (DE3) cultures harboring the empty pET29a vector were used as negative controls. A single L1 larva was then added to 24 wells. C0756, a Bacillus thuringiensis strain known to express Cry proteins, was used as a positive control. The plates were sealed and maintained at ambient laboratory conditions regarding temperature, lighting, and relative humidity. Mortality and stunting were assessed on day 7. This method was also used for bioassay evaluation of the purified Mv6_CRW_v1 protein against selected Lepidopteran species. As indicated in Tables 13 and 14, Mv6_CRW_v1 shows low to no insecticidal effect on the lepidopteran pests tested.

TABLE 13
Insecticidal activity of Mv6_CRW_v1 lysates
against Fall armyworm and Soybean Looper

| | Fall armyworm | |
|---|---|---|
| | (FAW) | Day 7 |
| Treatment | Total | Mortality |
| BL21*/pET29a-empty | 23 | − |
| BL21*/pET29a- | 24 | + |
| Mv6_CRW_v1 | | |
| C0756 Bt strain (+) | 24 | +++ |
| | Soybean looper | |
| | (SBL) | Day 7 |
| Treatment | Total | Mortality |
| BL21*/pET29a-empty | 24 | − |
| BL21*/pET29a- | 24 | − |
| Mv6_CRW_v1 | | |
| C0756 Bt strain (+) | 23 | ++++ |

TABLE 14
Insecticidal activity of purified Mv6_CRW_v1
against a Lepidopteran pest panel

| | Black Cutworm | |
|---|---|---|
| | (BCW) | Day 7 |
| Treatment | Total | Mortality |
| 50 mM Tris pH 8.0, 50 mM | 24 | − |
| NaCl | | |
| Mv6_CRW_v1 4 μg/cm2 | 24 | − |
| Mv6_CRW_v1 2 μg/cm2 | 24 | − |
| Mv6_CRW_v1 1 μg/cm2 | 24 | − |
| | European corn | |
| | borer (ECB) | Day 7 |
| Treatment | Total | Mortality |
| 50 mM Tris pH 8.0, 50 mM | 24 | + |
| NaCl | | |
| Mv6_CRW_v1 4 μg/cm2 | 24 | + |
| Mv6_CRW_v1 2 μg/cm2 | 24 | − |
| Mv6_CRW_v1 1 μg/cm2 | 24 | − |
| | Southwestern | |
| | corn borer | |
| | (SWCB) | Day 7 |
| Treatment | Total | Mortality |
| 50 mM Tris pH 8.0, 50 mM | 24 | − |
| NaCl | | |
| Mv6_CRW_v1 4 μg/cm2 | 24 | − |
| Mv6_CRW_v1 2 μg/cm2 | 23 | − |
| Mv6_CRW_v1 1 μg/cm2 | 24 | + |
| | Velvet bean | |
| | caterpillar (VBC) | Day 7 |
| Treatment | Total | Mortality |
| 50 mM Tris pH 8.0, 50 mM | 24 | + |
| NaCl | | |
| Mv6_CRW_v1 4 μg/cm2 | 24 | ++ |
| Mv6_CRW_v1 2 μg/cm2 | 24 | − |
| Mv6_CRW_v1 1 μg/cm2 | 24 | + |
| | Corn earworm | |
| | (CEW) | Day 7 |
| Treatment | Total | Mortality |
| 50 mM Tris pH 8.0, 50 mM | 24 | − |
| NaCl | | |
| Mv6_CRW_v1 4 μg/cm2 | 24 | + |
| Mv6_CRW_v1 2 μg/cm2 | 24 | + |
| | Soybean looper | |
| | (SBL) | Day 7 |
| Treatment | Total | Mortality |
| 50 mM Tris pH 8.0, 50 mM | 24 | + |
| NaCl | | |
| Mv6_CRW_v1 4 μg/cm2 | 24 | + |
| Mv6_CRW_v1 2 μg/cm2 | 24 | + |
| | Fall armyworm | |
| | (FAW) | Day 7 |
| Treatment | Total | Mortality |
| 50 mM Tris pH 8.0, 50 mM | 24 | − |
| NaCl | | |
| Mv6_CRW_v1 4 μg/cm2 | 24 | − |
| Mv6_CRW_v1 2 μg/cm2 | 24 | + |

### Example 10: Alanine Scanning Site-Directed Mutagenesis of Mv6_CRW_v1

Mutations were introduced along the entire length of Mv6 using alanine scanning site-directed mutagenesis to identify residues that influence bioactivity to WCRW. Each Alanine mutant was expressed using a pET29a construct. An E. coli BL21* lysate (derived from a 100 mL LB culture) was generated for each alanine mutant. Lysates were de-salted prior to being tested using diet-incorporation bioassay as described in Example 1. The bioassay results for the alanine scan mutants are shown in Table 15. As shown, changing certain residues in the Mv6 protein sequence reduced or eliminated the CRW bioactivity of Mv6. Residue changes that resulted in a decrease of the CRW mortality score to + or lower include D3A, W9A, I13A, R14A, L20A, E26A, G28A, I33A, L34A, V35A, D45A, F47A, E48A, L74A, L77A, E91A, L128A, H130A, L131A, R135A, E136A, R137A, L144A, Y145A, E147A, W153A, Y164A, P170A, W179A, D211A, M242A, D245A, M250A, Y260A, L264A, F279A, G285A, P286A, Y287A, I309A, D315A, V326A, I334A, D335A, Y341A, G346A, G355A, F374A, Y387A, G410A, and Y414A.

TABLE 15
Insecticidal activity of lysates of Mv6_CRW_v1 alanine scan mutants against WCR

| | SEQ ID | | | SEQ ID | |
|---|---|---|---|---|---|
| Alanine Scan Variant | NO: | Mortality | Alanine Scan Variant | NO: | Mortality |
| Mv6_CRW_v1 A2S | 30 | ++++ | Mv6_CRW_v1 N101A | 129 | ++++ |
| Mv6_CRW_v1 D3A | 31 | + | Mv6_CRW_v1 E102A | 130 | ++++ |
| Mv6_CRW_v1 L4A | 32 | ++++ | Mv6_CRW_v1 A103S | 131 | ++++ |
| Mv6_CRW_v1 V5A | 33 | ++++ | Mv6_CRW_v1 D104A | 132 | +++ |
| Mv6_CRW_v1 A6S | 34 | ++++ | Mv6_CRW_v1 R105A | 133 | ++++ |
| Mv6_CRW_v1 S7A | 35 | ++ | Mv6_CRW_v1 L106A | 134 | ++ |
| Mv6_CRW_v1 N8A | 36 | ++ | Mv6_CRW_v1 S107A | 135 | ++++ |
| Mv6_CRW_v1 W9A | 37 | − | Mv6_CRW_v1 V108A | 136 | ++++ |
| Mv6_CRW_v1 N10A | 38 | ++ | Mv6_CRW_v1 I109A | 137 | ++++ |
| Mv6_CRW_v1 N11A | 39 | ++ | Mv6_CRW_v1 F110A | 138 | ++ |
| Mv6_CRW_v1 R12A | 40 | ++ | Mv6_CRW_v1 R111A | 139 | ++ |
| Mv6_CRW_v1 I13A | 41 | + | Mv6_CRW_v1 Q112A | 140 | ++++ |
| Mv6_CRW_v1 R14A | 42 | + | Mv6_CRW_v1 S113A | 141 | +++ |
| Mv6_CRW_v1 D15A | 43 | ++++ | Mv6_CRW_v1 A114S | 142 | ++++ |
| Mv6_CRW_v1 A16S | 44 | ++++ | Mv6_CRW_v1 N115A | 143 | ++ |
| Mv6_CRW_v1 V17A | 45 | +++ | Mv6_CRW_v1 N116A | 144 | ++++ |
| Mv6_CRW_v1 I18A | 46 | ++++ | Mv6_CRW_v1 I117A | 145 | +++ |
| Mv6_CRW_v1 K19A | 47 | ++++ | Mv6_CRW_v1 H118A | 146 | ++++ |
| Mv6_CRW_v1 L20A | 48 | + | Mv6_CRW_v1 L119A | 147 | ++ |
| Mv6_CRW_v1 S21A | 49 | ++++ | Mv6_CRW_v1 I120A | 148 | ++++ |
| Mv6_CRW_v1 S22A | 50 | ++++ | Mv6_CRW_v1 Q121A | 149 | ++++ |
| Mv6_CRW_v1 K23A | 51 | +++ | Mv6_CRW_v1 L122A | 150 | ++++ |
| Mv6_CRW_v1 I24A | 52 | +++ | Mv6_CRW_v1 V123A | 151 | ++++ |
| Mv6_CRW_v1 P25A | 53 | +++ | Mv6_CRW_v1 I124A | 152 | ++++ |
| Mv6_CRW_v1 E26A | 54 | + | Mv6_CRW_v1 S125A | 153 | ++++ |
| Mv6_CRW_v1 V27A | 55 | ++++ | Mv6_CRW_v1 L126A | 154 | ++ |
| Mv6_CRW_v1 G28A | 56 | + | Mv6_CRW_v1 A127S | 155 | ++++ |
| Mv6_CRW_v1 T29A | 57 | ++++ | Mv6_CRW_v1 L128A | 156 | + |
| Mv6_CRW_v1 A30S | 58 | ++++ | Mv6_CRW_v1 L129A | 157 | ++ |
| Mv6_CRW_v1 V31A | 59 | ++++ | Mv6_CRW_v1 H130A | 158 | + |
| Mv6_CRW_v1 G32A | 60 | ++++ | Mv6_CRW_v1 L131A | 159 | + |
| Mv6_CRW_v1 I33A | 61 | + | Mv6_CRW_v1 T132A | 160 | ++++ |
| Mv6_CRW_v1 L34A | 62 | + | Mv6_CRW_v1 A133S | 161 | +++ |
| Mv6_CRW_v1 V35A | 63 | + | Mv6_CRW_v1 L134A | 162 | + |
| Mv6_CRW_v1 G36A | 64 | ++++ | Mv6_CRW_v1 R135A | 163 | + |
| Mv6_CRW_v1 F37A | 65 | ++++ | Mv6_CRW_v1 E136A | 164 | + |
| Mv6_CRW_v1 F38A | 66 | + | Mv6_CRW_v1 R137A | 165 | + |
| Mv6_CRW_v1 W39A | 67 | ++ | Mv6_CRW_v1 F138A | 166 | ++++ |
| Mv6_CRW_v1 P40A | 68 | + | Mv6_CRW_v1 D139A | 167 | ++++ |
| Mv6_CRW_v1 Q41A | 69 | ++++ | Mv6_CRW_v1 F140A | 168 | + |
| Mv6_CRW_v1 D42A | 70 | ++++ | Mv6_CRW_v1 G141A | 169 | ++ |
| Mv6_CRW_v1 K43A | 71 | ++++ | Mv6_CRW_v1 R142A | 170 | ++++ |
| Mv6_CRW_v1 V44A | 72 | ++++ | Mv6_CRW_v1 S143A | 171 | ++++ |
| Mv6_CRW_v1 D45A | 73 | + | Mv6_CRW_v1 L144A | 172 | + |
| Mv6_CRW_v1 I46A | 74 | ++ | Mv6_CRW_v1 Y145A | 173 | + |
| Mv6_CRW_v1 F47A | 75 | + | Mv6_CRW_v1 S146A | 174 | ++++ |
| Mv6_CRW_v1 E48A | 76 | + | Mv6_CRW_v1 E147A | 175 | + |
| Mv6_CRW_v1 S49A | 77 | ++++ | Mv6_CRW_v1 D148A | 176 | +++ |
| Mv6_CRW_v1 I50A | 78 | ++++ | Mv6_CRW_v1 W153A | 181 | + |
| Mv6_CRW_v1 K51A | 79 | ++++ | Mv6_CRW_v1 K154A | 182 | ++++ |
| Mv6_CRW_v1 A52S | 80 | ++++ | Mv6_CRW_v1 E155A | 183 | ++++ |
| Mv6_CRW_v1 D53A | 81 | ++ | Mv6_CRW_v1 D156A | 184 | ++++ |
| Mv6_CRW_v1 I54A | 82 | +++ | Mv6_CRW_v1 L157A | 185 | +++ |
| Mv6_CRW_v1 T55A | 83 | ++++ | Mv6_CRW_v1 E158A | 186 | +++ |
| Mv6_CRW_v1 L57A | 85 | ++ | Mv6_CRW_v1 N159A | 187 | ++++ |
| Mv6_CRW_v1 V58A | 86 | ++++ | Mv6_CRW_v1 M160A | 188 | ++++ |
| Mv6_CRW_v1 K59A | 87 | ++++ | Mv6_CRW_v1 Y161A | 189 | ++ |
| Mv6_CRW_v1 K60A | 88 | ++++ | Mv6_CRW_v1 K162A | 190 | ++++ |
| Mv6_CRW_v1 E61A | 89 | +++ | Mv6_CRW_v1 T163A | 191 | ++++ |
| Mv6_CRW_v1 I62A | 90 | ++ | Mv6_CRW_v1 Y164A | 192 | + |
| Mv6_CRW_v1 L63A | 91 | ++ | Mv6_CRW_v1 I165A | 193 | +++ |
| Mv6_CRW_v1 D64A | 92 | ++++ | Mv6_CRW_v1 V166A | 194 | ++++ |
| Mv6_CRW_v1 Y65A | 93 | ++++ | Mv6_CRW_v1 D167A | 195 | +++ |
| Mv6_CRW_v1 E66A | 94 | +++ | Mv6_CRW_v1 F168A | 196 | +++ |
| Mv6_CRW_v1 L67A | 95 | + | Mv6_CRW_v1 I169A | 197 | +++ |
| Mv6_CRW_v1 S68A | 96 | ++++ | Mv6_CRW_v1 P170A | 198 | + |
| Mv6_CRW_v1 L69A | 97 | ++++ | Mv6_CRW_v1 D171A | 199 | ++++ |
| Mv6_CRW_v1 H70A | 98 | +++ | Mv6_CRW_v1 I172A | 200 | ++++ |
| Mv6_CRW_v1 K71A | 99 | ++++ | Mv6_CRW_v1 Y173A | 201 | ++ |
| Mv6_CRW_v1 N72A | 100 | ++++ | Mv6_CRW_v1 K174A | 202 | ++++ |
| Mv6_CRW_v1 E73A | 101 | ++++ | Mv6_CRW_v1 K175A | 203 | ++++ |
| Mv6_CRW_v1 L74A | 102 | + | Mv6_CRW_v1 W176A | 204 | ++ |
| Mv6_CRW_v1 D75A | 103 | ++++ | Mv6_CRW_v1 K177A | 205 | ++++ |
| Mv6_CRW_v1 A76S | 104 | ++++ | Mv6_CRW_v1 E178A | 206 | ++++ |
| Mv6_CRW_v1 L77A | 105 | + | Mv6_CRW_v1 W179A | 207 | + |
| Mv6_CRW_v1 K78A | 106 | ++++ | Mv6_CRW_v1 P194A | 222 | ++++ |
| Mv6_CRW_v1 E79A | 107 | ++++ | Mv6_CRW_v1 I195A | 223 | ++++ |
| Mv6_CRW_v1 T80A | 108 | ++++ | Mv6_CRW_v1 T196A | 224 | ++++ |
| Mv6_CRW_v1 M81A | 109 | ++ | Mv6_CRW_v1 L197A | 225 | ++++ |
| Mv6_CRW_v1 K82A | 110 | ++++ | Mv6_CRW_v1 P198A | 226 | ++++ |
| Mv6_CRW_v1 R83A | 111 | ++++ | Mv6_CRW_v1 P199A | 227 | ++ |
| Mv6_CRW_v1 Y84A | 112 | ++ | Mv6_CRW_v1 F200A | 228 | ++ |
| Mv6_CRW_v1 E85A | 113 | ++++ | Mv6_CRW_v1 Y201A | 229 | ++++ |
| Mv6_CRW_v1 T86A | 114 | ++++ | Mv6_CRW_v1 T202A | 230 | ++++ |
| Mv6_CRW_v1 A87S | 115 | ++++ | Mv6_CRW_v1 N203A | 231 | ++++ |
| Mv6_CRW_v1 M88A | 116 | ++++ | Mv6_CRW_v1 E204A | 232 | ++++ |
| Mv6_CRW_v1 N89A | 117 | ++++ | Mv6_CRW_v1 S205A | 233 | ++++ |
| Mv6_CRW_v1 H90A | 118 | ++++ | Mv6_CRW_v1 H206A | 234 | ++++ |
| Mv6_CRW_v1 E91A | 119 | + | Mv6_CRW_v1 A207S | 235 | ++++ |
| Mv6_CRW_v1 K92A | 120 | ++++ | Mv6_CRW_v1 R208A | 236 | ++++ |
| Mv6_CRW_v1 G93A | 121 | ++++ | Mv6_CRW_v1 V209A | 237 | +++ |
| Mv6_CRW_v1 L94A | 122 | ++++ | Mv6_CRW_v1 V210A | 238 | ++++ |
| Mv6_CRW_v1 F95A | 123 | ++++ | Mv6_CRW_v1 D211A | 239 | + |
| Mv6_CRW_v1 L96A | 124 | + | Mv6_CRW_v1 N212A | 240 | ++++ |
| Mv6_CRW_v1 N97A | 125 | ++++ | Mv6_CRW_v1 V213A | 241 | ++ |
| Mv6_CRW_v1 S98A | 126 | ++++ | Mv6_CRW_v1 T214A | 242 | ++++ |
| Mv6_CRW_v1 W99A | 127 | +++ | Mv6_CRW_v1 G215A | 243 | ++++ |
| Mv6_CRW_v1 I100A | 128 | ++ | Mv6_CRW_v1 E216A | 244 | ++++ |
| | SEQ ID | | | SEQ ID | |
| Alanine Scan Variant | NO: | Mortality | Alanine Scan Variant | NO: | Mortality |
| Mv6_CRW_v1 I217A | 245 | ++++ | Mv6_CRW_v1 P352A | 380 | ++++ |
| Mv6_CRW_v1 K218A | 246 | ++++ | Mv6_CRW_v1 S353A | 381 | ++++ |
| Mv6_CRW_v1 N219A | 247 | ++++ | Mv6_CRW_v1 G354A | 382 | ++++ |
| Mv6_CRW_v1 F220A | 248 | ++++ | Mv6_CRW_v1 G355A | 383 | + |
| Mv6_CRW_v1 Y221A | 249 | ++++ | Mv6_CRW_v1 L356A | 384 | ++++ |
| Mv6_CRW_v1 Q222A | 250 | ++++ | Mv6_CRW_v1 Q357A | 385 | ++++ |
| Mv6_CRW_v1 D223A | 251 | ++++ | Mv6_CRW_v1 H358A | 386 | ++++ |
| Mv6_CRW_v1 L224A | 252 | ++++ | Mv6_CRW_v1 D359A | 387 | ++++ |
| Mv6_CRW_v1 S225A | 253 | ++++ | Mv6_CRW_v1 I360A | 388 | ++++ |
| Mv6_CRW_v1 S226A | 254 | ++++ | Mv6_CRW_v1 D361A | 389 | ++++ |
| Mv6_CRW_v1 S227A | 255 | ++++ | Mv6_CRW_v1 V362A | 390 | ++++ |
| Mv6_CRW_v1 T240A | 268 | ++++ | Mv6_CRW_v1 S363A | 391 | ++++ |
| Mv6_CRW_v1 M242A | 270 | + | Mv6_CRW_v1 N364A | 392 | ++++ |
| Mv6_CRW_v1 C243A | 271 | ++++ | Mv6_CRW_v1 K365A | 393 | ++++ |
| Mv6_CRW_v1 N244A | 272 | ++ | Mv6_CRW_v1 L366A | 394 | ++++ |
| Mv6_CRW_v1 D245A | 273 | + | Mv6_CRW_v1 I367A | 395 | ++++ |
| Mv6_CRW_v1 A246S | 274 | +++ | Mv6_CRW_v1 H368A | 396 | ++++ |
| Mv6_CRW_v1 Y247A | 275 | ++++ | Mv6_CRW_v1 G369A | 397 | ++++ |
| Mv6_CRW_v1 C248A | 276 | ++++ | Mv6_CRW_v1 F370A | 398 | ++++ |
| Mv6_CRW_v1 D249A | 277 | ++++ | Mv6_CRW_v1 H371A | 399 | ++++ |
| Mv6_CRW_v1 M250A | 278 | + | Mv6_CRW_v1 L372A | 400 | +++ |
| Mv6_CRW_v1 V251A | 279 | ++++ | Mv6_CRW_v1 G373A | 401 | ++++ |
| Mv6_CRW_v1 G252A | 280 | ++++ | Mv6_CRW_v1 F374A | 402 | + |
| Mv6_CRW_v1 S253A | 281 | ++++ | Mv6_CRW_v1 S375A | 403 | ++++ |
| Mv6_CRW_v1 F258A | 286 | ++ | Mv6_CRW_v1 S376A | 404 | ++++ |
| Mv6_CRW_v1 Y260A | 288 | + | Mv6_CRW_v1 G377A | 405 | ++++ |
| Mv6_CRW_v1 L261A | 289 | ++++ | Mv6_CRW_v1 V378A | 406 | ++++ |
| Mv6_CRW_v1 Q262A | 290 | ++++ | Mv6_CRW_v1 L379A | 407 | +++ |
| Mv6_CRW_v1 I263A | 291 | ++ | Mv6_CRW_v1 A380S | 408 | ++++ |
| Mv6_CRW_v1 L264A | 292 | + | Mv6_CRW_v1 R381A | 409 | ++++ |
| Mv6_CRW_v1 P265A | 293 | ++++ | Mv6_CRW_v1 I382A | 410 | ++++ |
| Mv6_CRW_v1 D266A | 294 | ++++ | Mv6_CRW_v1 K383A | 411 | ++++ |
| Mv6_CRW_v1 N267A | 295 | ++++ | Mv6_CRW_v1 V384A | 412 | ++++ |
| Mv6_CRW_v1 N268A | 296 | ++++ | Mv6_CRW_v1 D385A | 413 | ++++ |
| Mv6_CRW_v1 K269A | 297 | ++ | Mv6_CRW_v1 Y386A | 414 | +++ |
| Mv6_CRW_v1 N270A | 298 | ++++ | Mv6_CRW_v1 Y387A | 415 | + |
| Mv6_CRW_v1 F271A | 299 | ++ | Mv6_CRW_v1 D388A | 416 | +++ |
| Mv6_CRW_v1 A272S | 300 | ++++ | Mv6_CRW_v1 G389A | 417 | ++++ |
| Mv6_CRW_v1 K273A | 301 | ++++ | Mv6_CRW_v1 T390A | 418 | ++++ |
| Mv6_CRW_v1 Y274A | 302 | + | Mv6_CRW_v1 S391A | 419 | ++++ |
| Mv6_CRW_v1 V278A | 306 | ++++ | Mv6_CRW_v1 S392A | 420 | ++++ |
| Mv6_CRW_v1 F279A | 307 | + | Mv6_CRW_v1 D393A | 421 | ++++ |
| Mv6_CRW_v1 G280A | 308 | ++++ | Mv6_CRW_v1 M394A | 422 | ++++ |
| Mv6_CRW_v1 E281A | 309 | ++++ | Mv6_CRW_v1 L395A | 423 | ++++ |
| Mv6_CRW_v1 V282A | 310 | ++++ | Mv6_CRW_v1 G396A | 424 | ++++ |
| Mv6_CRW_v1 F283A | 311 | ++++ | Mv6_CRW_v1 N397A | 425 | ++++ |
| Mv6_CRW_v1 K284A | 312 | ++++ | Mv6_CRW_v1 R398A | 426 | ++++ |
| Mv6_CRW_v1 G285A | 313 | + | Mv6_CRW_v1 G399A | 427 | ++++ |
| Mv6_CRW_v1 P286A | 314 | + | Mv6_CRW_v1 G400A | 428 | ++++ |
| Mv6_CRW_v1 Y287A | 315 | + | Mv6_CRW_v1 W401A | 429 | ++++ |
| Mv6_CRW_v1 S288A | 316 | ++++ | Mv6_CRW_v1 P402A | 430 | ++++ |
| Mv6_CRW_v1 L289A | 317 | ++++ | Mv6_CRW_v1 I403A | 431 | ++++ |
| Mv6_CRW_v1 D290A | 318 | +++ | Mv6_CRW_v1 T404A | 432 | ++++ |
| Mv6_CRW_v1 L291A | 319 | ++++ | Mv6_CRW_v1 E405A | 433 | ++++ |
| Mv6_CRW_v1 S292A | 320 | ++++ | Mv6_CRW_v1 Q406A | 434 | ++++ |
| Mv6_CRW_v1 Y293A | 321 | ++++ | Mv6_CRW_v1 V407A | 435 | ++++ |
| Mv6_CRW_v1 S294A | 322 | ++++ | Mv6_CRW_v1 A408S | 436 | ++++ |
| Mv6_CRW_v1 N295A | 323 | ++++ | Mv6_CRW_v1 K409A | 437 | ++++ |
| Mv6_CRW_v1 Y296A | 324 | ++++ | Mv6_CRW_v1 G410A | 438 | + |
| Mv6_CRW_v1 S297A | 325 | ++++ | Mv6_CRW_v1 P411A | 439 | ++ |
| Mv6_CRW_v1 Y298A | 326 | ++++ | Mv6_CRW_v1 T412A | 440 | ++++ |
| Mv6_CRW_v1 G299A | 327 | ++++ | Mv6_CRW_v1 L413A | 441 | ++++ |
| Mv6_CRW_v1 Y300A | 328 | ++++ | Mv6_CRW_v1 Y414A | 442 | + |
| Mv6_CRW_v1 G301A | 329 | ++++ | Mv6_CRW_v1 K415A | 443 | ++++ |
| Mv6_CRW_v1 N302A | 330 | +++ | Mv6_CRW_v1 V416A | 444 | ++++ |
| Mv6_CRW_v1 N303A | 331 | ++++ | Mv6_CRW_v1 I417A | 445 | ++++ |
| Mv6_CRW_v1 V304A | 332 | ++++ | Mv6_CRW_v1 S418A | 446 | ++++ |
| Mv6_CRW_v1 P305A | 333 | +++ | Mv6_CRW_v1 W419A | 447 | ++++ |
| Mv6_CRW_v1 S306A | 334 | ++++ | Mv6_CRW_v1 S420A | 448 | ++++ |
| Mv6_CRW_v1 F307A | 335 | +++ | Mv6_CRW_v1 Y421A | 449 | ++++ |
| Mv6_CRW_v1 R308A | 336 | ++++ | Mv6_CRW_v1 K422A | 450 | ++++ |
| Mv6_CRW_v1 I309A | 337 | + | Mv6_CRW_v1 V423A | 451 | ++++ |
| Mv6_CRW_v1 N310A | 338 | ++++ | Mv6_CRW_v1 D424A | 452 | ++++ |
| Mv6_CRW_v1 A320S | 348 | ++++ | Mv6_CRW_v1 Y425A | 453 | ++++ |
| Mv6_CRW_v1 G321A | 349 | ++++ | Mv6_CRW_v1 G426A | 454 | ++++ |
| Mv6_CRW_v1 P322A | 350 | ++++ | Mv6_CRW_v1 P427A | 455 | ++++ |
| Mv6_CRW_v1 I323A | 351 | ++++ | Mv6_CRW_v1 S428A | 456 | ++++ |
| Mv6_CRW_v1 T324A | 352 | ++++ | Mv6_CRW_v1 G429A | 457 | ++++ |
| Mv6_CRW_v1 K325A | 353 | ++++ | Mv6_CRW_v1 T430A | 458 | ++++ |
| Mv6_CRW_v1 V326A | 354 | − | Mv6_CRW_v1 Y431A | 459 | ++++ |
| Mv6_CRW_v1 I327A | 355 | ++++ | Mv6_CRW_v1 G432A | 460 | ++++ |
| Mv6_CRW_v1 I328A | 356 | ++++ | Mv6_CRW_v1 P433A | 461 | ++++ |
| Mv6_CRW_v1 R329A | 357 | ++++ | Mv6_CRW_v1 S434A | 462 | ++++ |
| Mv6_CRW_v1 E330A | 358 | ++++ | Mv6_CRW_v1 V435A | 463 | ++++ |
| Mv6_CRW_v1 W331A | 359 | +++ | Mv6_CRW_v1 I436A | 464 | ++++ |
| Mv6_CRW_v1 N332A | 360 | ++ | Mv6_CRW_v1 Q437A | 465 | ++++ |
| Mv6_CRW_v1 S333A | 361 | ++++ | Mv6_CRW_v1 L438A | 466 | ++++ |
| Mv6_CRW_v1 I334A | 362 | + | Mv6_CRW_v1 Q439A | 467 | ++++ |
| Mv6_CRW_v1 D335A | 363 | − | Mv6_CRW_v1 Y440A | 468 | + |
| Mv6_CRW_v1 A336S | 364 | ++++ | Mv6_CRW_v1 T441A | 469 | ++++ |
| Mv6_CRW_v1 M337A | 365 | ++++ | Mv6_CRW_v1 P442A | 470 | +++ |
| Mv6_CRW_v1 Q338A | 366 | ++++ | Mv6_CRW_v1 H443A | 471 | ++++ |
| Mv6_CRW_v1 F339A | 367 | +++ | Mv6_CRW_v1 M444A | 472 | ++++ |
| Mv6_CRW_v1 I340A | 368 | ++++ | Mv6_CRW_v1 D445A | 473 | ++++ |
| Mv6_CRW_v1 Y341A | 369 | + | Mv6_CRW_v1 N149A | 177 | ++++ |
| Mv6_CRW_v1 Q342A | 370 | ++++ | Mv6_CRW_v1 T150A | 178 | +++ |
| Mv6_CRW_v1 G350A | 378 | ++ | Mv6_CRW_v1 P151A | 179 | ++++ |
| Mv6_CRW_v1 N351A | 379 | ++++ | Mv6_CRW_v1 Q152A | 180 | ++++ |

| | SEQ ID | |
|---|---|---|
| Alanine Scan Variant | NO: | Mortality |
| Mv6_CRW_v1 R180A | 208 | + |
| Mv6_CRW_v1 T181A | 209 | ++++ |
| Mv6_CRW_v1 N182A | 210 | ++ |
| Mv6_CRW_v1 Q183A | 211 | +++ |
| Mv6_CRW_v1 V184A | 212 | ++ |
| Mv6_CRW_v1 E185A | 213 | ++++ |
| Mv6_CRW_v1 I186A | 214 | ++++ |
| Mv6_CRW_v1 S187A | 215 | ++++ |
| Mv6_CRW_v1 S188A | 216 | ++++ |
| Mv6_CRW_v1 W189A | 217 | ++ |
| Mv6_CRW_v1 L190A | 218 | ++++ |
| Mv6_CRW_v1 Q192A | 220 | ++++ |
| Mv6_CRW_v1 R193A | 221 | ++++ |
| Mv6_CRW_v1 Q236A | 264 | ++++ |
| Mv6_CRW_v1 A237S | 265 | ++ |
| Mv6_CRW_v1 H238A | 266 | +++ |
| Mv6_CRW_v1 K239A | 267 | ++ |
| Mv6_CRW_v1 R241A | 269 | ++ |
| Mv6_CRW_v1 S255A | 283 | ++++ |
| Mv6_CRW_v1 S259A | 287 | ++++ |
| Mv6_CRW_v1 D275A | 303 | ++ |
| Mv6_CRW_v1 E277A | 305 | ++++ |
| Mv6_CRW_v1 G343A | 371 | ++++ |
| Mv6_CRW_v1 R344A | 372 | ++++ |
| Mv6_CRW_v1 N345A | 373 | ++++ |
| Mv6_CRW_v1 G346A | 374 | + |
| Mv6_CRW_v1 S347A | 375 | ++++ |
| Mv6_CRW_v1 M348A | 376 | ++++ |
| Mv6_CRW_v1 A349S | 377 | ++++ |
| Mv6_CRW_v1 T230A | 258 | +++ |
| Mv6_CRW_v1 I234A | 262 | ++ |
| Mv6_CRW_v1 I254A | 282 | +++ |
| Mv6_CRW_v1 A256S | 284 | ++++ |
| Mv6_CRW_v1 T257A | 285 | +++ |
| Mv6_CRW_v1 K276A | 304 | ++++ |
| Mv6_CRW_v1 K56A | 84 | ++++ |
| Mv6_CRW_v1 T191A | 219 | ++++ |
| Mv6_CRW_v1 T228A | 256 | ++++ |
| Mv6_CRW_v1 S229A | 257 | +++ |
| Mv6_CRW_v1 F231A | 259 | ++ |
| Mv6_CRW_v1 N232A | 260 | +++ |
| Mv6_CRW_v1 N233A | 261 | +++ |
| Mv6_CRW_v1 C235A | 263 | ++++ |
| Mv6_CRW_v1 P311A | 339 | ++++ |
| Mv6_CRW_v1 T312A | 340 | ++++ |
| Mv6_CRW_v1 N313A | 341 | ++++ |
| Mv6_CRW_v1 N314A | 342 | +++ |
| Mv6_CRW_v1 D315A | 343 | + |
| Mv6_CRW_v1 Y316A | 344 | ++++ |
| Mv6_CRW_v1 S317A | 345 | ++++ |
| Mv6_CRW_v1 S318A | 346 | +++ |
| Mv6_CRW_v1 S319A | 347 | ++++ |

1. A nucleic acid molecule comprising a heterologous promoter operably linked to a nucleic acid sequence encoding a polypeptide having at least 80%, 90%, 95% or 100% identity to the amino acid sequence set forth in any one of SEQ ID NO: 1, 2-4, or 30-473, wherein said polypeptide has insecticidal activity.

2. The nucleic acid molecule of claim 1, wherein
a. said polypeptide comprises a transit peptide;
b. said polypeptide comprises a chloroplast transit peptide; or
c. said polypeptide comprises least 90%, 95% or 100% identity to the amino acid sequence set forth in any one of SEQ ID NO: 26, 27, 28 or 29; or
d. said heterologous promoter is a plant expressible promoter; or
e. the nucleic acid sequence encoding the polypeptide is codon optimized for expression in a plant.

3. (canceled)

4. (canceled)

5. A vector comprising the nucleic acid of claim 1.

6. A transgenic host cell comprising a heterologous polypeptide comprising an amino acid sequence having at least 80%, 90%, 95% or 100% sequence identity to any one of SEQ ID Nos: 1, 2-4, or 30-473, wherein said polypeptide has insecticidal activity.

7. The transgenic host cell of claim 6, wherein
a. said polypeptide further comprises a transit peptide;
b. said polypeptide further comprises a chloroplast transit peptide; or
c. said polypeptide comprises at least 90%, 95% or 100% identity to the amino acid sequence set forth in any one of SEQ ID NO: 26, 27, 28 or 29; or
d. the transgenic host cell is a bacterial cell; or
e. the bacterial cell is an Agrobacterium, Bacillus, or an Escherichia coli cell.

8. (canceled)

9. (canceled)

10. The transgenic host cell of claim 6, wherein
a. the host cell is a plant cell; or
b. the plant cell of (a) is a monocot cell; or
c. the plant cell of (b) is a maize cell.

11. (canceled)

12. (canceled)

13. (canceled)

14. A polypeptide comprising an amino acid sequence having at least 80%, 90%, 95% or 100% sequence identity to any one of SEQ ID Nos: 1, 2-4, or 30-473, wherein said polypeptide has insecticidal activity, and
a. wherein said amino sequence is operably linked to a heterologous transit peptide;
b. wherein said amino sequence is operably linked to a heterologous chloroplast transit peptide or
c. said polypeptide comprises least 90%, 95% or 100% identity to the amino acid sequence set forth in any one of SEQ ID NO: 26, 27, 28 or 29; or
d. said polypeptide is isolated.

15. A composition comprising the polypeptide of claim 14.

16. (canceled)

17. A plant comprising a polypeptide having an amino acid sequence with at least 80%, 90%, 95% or 100% sequence identity to any one of SEQ ID Nos: 1, 2-4, or 30-473, wherein said polypeptide has insecticidal activity, wherein said polypeptide is heterologous to said plant.

18. The plant of claim 17, wherein the polypeptide further comprises a
a. a transit peptide;
b. a chloroplast transit peptide; or
c. said polypeptide comprises least 90%, 95% or 100% identity to the amino acid sequence set forth in any one of SEQ ID NO: 26, 27, 28 or 29.

19. The plant of claim 17, wherein the polypeptide is encoded by a polynucleotide operably linked to a plant expressible promoter.

20. The plant of claim 19, wherein the polynucleotide operably linked to a plant expressible promoter is stably transformed into the genome of the plant.

21. The plant of claim 17, wherein the plant is a monocot or wherein the plant is a maize plant.

22. (canceled)

23. A seed of the plant of claim 20, wherein said polynucleotide operably linked to the plant expressible promoter is stably transformed into the genome of the seed.

24. The seed of claim 23, wherein said seed is a monocot or wherein said seed is maize.

25. (canceled)

26. A method of producing a transgenic plant, the method comprising:
a. introducing into a plant cell the nucleic acid molecule of claim 1;
b. selecting a plant cell comprising the nucleic acid molecule; and
c. regenerating a plant from the selected plant cell.

27. A method for producing a transgenic plant, the method comprising:
a. sexually crossing a first parent plant with a second parent plant, wherein the first or the second parent plant is the plant of claim 20; and
b. selecting a progeny plant with enhanced insecticidal properties, wherein the selected progeny plant comprises the polynucleotide.

28. (canceled)

29. A method of controlling a coleopteran pest comprising delivering to the pest or an environment thereof the polypeptide of claim 13.

30. The method of claim 29, wherein the polypeptide is delivered by feeding.

31. The method of claim 30, wherein the feeding comprises the pest feeding on a plant part that comprises the polypeptide.

32. A method of controlling a coleopteran pest comprising growing in an area of cultivation the plant of claim 17 and controlling the population of coleopteran pests in the area of cultivation.

33. (canceled)

34. (canceled)

35. (canceled)

36. (canceled)

37. (canceled)

38. (canceled)

39. (canceled)
